(19) United States
(12) Patent Application Publication
(10) Pub. No.: US 2013/0130102 A1
(43) Pub. Date: May 23, 2013

(54) ETHER COMPOUND, ELECTROLYTE COMPOSITION FOR NON-AQUEOUS BATTERY, BINDER COMPOSITION FOR NON-AQUEOUS BATTERY ELECTRODE, SLURRY COMPOSITION FOR NON-AQUEOUS BATTERY ELECTRODE, ELECTRODE FOR NON-AQUEOUS BATTERY AND NON-AQUEOUS BATTERY

(71) Applicants: Kei Sakamoto, Tokyo (JP); Natsuko Nakata, Tokyo (JP); Kumi Okuyama, Tokyo (JP)

(72) Inventors: Kei Sakamoto, Tokyo (JP); Natsuko Nakata, Tokyo (JP); Kumi Okuyama, Tokyo (JP)

(73) Assignee: ZEON CORPORATION, Tokyo (JP)

(21) Appl. No.: 13/812,938

(22) PCT Filed: Jul. 22, 2011
(86) PCT No.: PCT/JP2011/066745

§ 371 Date: Jan. 29, 2013

(30) Foreign Application Priority Data
Jul. 30, 2010 (JP) 2010-171859

(51) Int. Cl. H01M 4/60; H01M 10/052; H01M 10/0564; C07D 307/12

(52) U.S. Cl. CPC H01M 4/60; C07D 307/12; H01M 10/052; H01M 10/0564
USPC 429/200; 549/497; 524/111; 429/188; 429/199; 429/211; 252/182.1

(57) ABSTRACT

An ether compound represented by the following formula and its use:

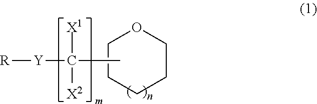

wherein n represents 0 or 1, m represents an integer of 0 to 2, Y represents —O—, —S—, —C(═O)—O—, and —O—C(═O)—, X¹ and X² represent a hydrogen atom or a fluorine atom, and R represents an aliphatic hydrocarbon group having 1 to 20 carbon atoms and substituted with one or more fluorine atoms, with a proviso that, when m is 0, R has 3 to 20 carbon atoms. R may have in the bond thereof intervening oxygen atom, sulfur atom, and carbonyl group.

### FIELD

The present invention relates to a novel ether compound as well as an electrolyte solution composition for a non-aqueous battery, a binder composition for a non-aqueous battery electrode, a slurry composition for a non-aqueous battery electrode, an electrode for a non-aqueous battery, and a non-aqueous battery, each of which uses the novel ether compound.

### BACKGROUND

Non-aqueous batteries such as lithium secondary batteries are being put into practical use in a wide range of applications from customer power sources for, e.g., cell phones and notebook computers to drive power sources equipped on vehicles such as automobiles. Important properties required for non-aqueous batteries such as lithium secondary batteries may include high discharge capacity and stable charge-discharge cycle. The stable charge-discharge cycle herein means that the discharge capacity of the non-aqueous battery does not easily decrease even after repeated charging and discharging. Further, such an excellence stability of the charge-discharge cycle may be also referred to as excellent cycle property.

It has been conventionally known that the composition of the non-aqueous electrolyte solution has a significant influence on the stability of the charge-discharge cycle of the non-aqueous batteries such as lithium secondary batteries. Therefore, there has been proposed a technology for improving performance of a non-aqueous battery by developing the composition of the non-aqueous electrolyte solution. For example, Patent Literature 1 proposes an electrolyte solution in which lithium trifluoromethanesulfonate is dissolved as an electrolyte in a mixed solvent containing specific amounts of a cyclic carbonate ester, a chain carbonate ester, and an ether. Further, Patent Literature 2 proposes a non-aqueous battery in which a metal composite oxide having a high discharge capacity is used as a negative electrode and a mixed solvent of, e.g., ethylene carbonate and a chain carbonate ester is used as a non-aqueous electrolyte. Patent Literatures 3 and 4 describe technologies of adding a simple cyclic ether compound such as 1,3-dioxolane, tetrahydrofuran, tetrahydropyran, and dioxane to a non-aqueous electrolyte solution.

### CITATION LIST

### Patent Literature

Patent Literature 1: Japanese Patent Application Laid-Open No. Hei. 8-64240 A (corresponding publication: U.S. Pat. No. 4,525,985)
Patent Literature 2: Japanese Patent Application Laid-Open No. Hei. 8-130036 A
Patent Literature 3: Japanese Patent Application Laid-Open No. Hei. 10-116631 A
Patent Literature 4: Japanese Patent Application Laid-Open No. 2006-012780 A (corresponding publication: Description of European Patent Publication No. 1744394 A)

### SUMMARY

### Technical Problem

However, in the technologies described in Patent Literatures 1 and 2, although the stability of the charge-discharge cycle was improved, the discharge capacity that an electrode material intrinsically has was decreased and it was thus unable to obtain a sufficient discharge capacity.

Further, Patent Literature 4 describes that the technologies of adding a simple cyclic ether compound described in Patent Literatures 3 and 4 do not improve continuous charge property (particularly, a remaining capacity after continuous charge) and high-temperature storage property. In particular, the problem in a high temperature environment has been left unresolved.

The present invention has been devised in view of the aforementioned problem. An object of the present invention is to provide a non-aqueous battery which has a high capacity and an excellent stability of a charge-discharge cycle at high temperatures by balancing a high discharge capacity and a stable charge-discharge cycle in a high temperature environment.

### Solution to Problem

As a result of extensive studies in order to achieve the aforementioned object, the present inventors have devised a novel ether compound. The inventors have found out that a high discharge capacity and a stable charge-discharge cycle in a high temperature environment of a non-aqueous battery can be achieved at high levels in a balanced manner by providing an electrolyte solution composition for anon-aqueous battery electrode, a binder composition for a non-aqueous battery electrode, a slurry composition for a non-aqueous battery electrode, or an electrode for a non-aqueous battery, each of which utilizes the ether compound, to a non-aqueous battery. Thus, the present invention has been completed.

That is, according to the present invention, the following [1] to [9] are provided.

(1) An ether compound represented by the following formula (1),

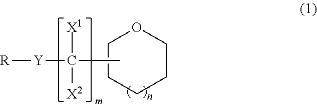

wherein

n represents 0 or 1,

m represents an integer of 0 to 2,

Y represents any one selected from the group consisting of —O—, —S—, —C(═O)—O—, and —O—C(═O)—,

X¹ and X² each independently represent a hydrogen atom or a fluorine atom, and

R represents an aliphatic hydrocarbon group having 1 to 20 carbon atoms and substituted with one or more fluorine atoms, with a proviso that, when m is 0, R has 3 to 20 carbon atoms; wherein R may have in the bond thereof one or more intervening moieties selected from the group consisting of an oxygen atom, a sulfur atom, and a carbonyl group.

(2) An ether compound represented by the following formula (2),

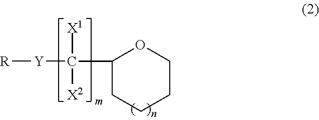

wherein

n represents 0 or 1,

m represents an integer of 0 to 2,

Y represents any one selected from the group consisting of —O—, —S—, —C(═O)—O—, and —O—C(═O)—,

X¹ and X² each independently represent a hydrogen atom or a fluorine atom, and

R represents an aliphatic hydrocarbon group having 1 to 20 carbon atoms and substituted with one or more fluorine atoms, with a proviso that, when m is 0, R has 3 to 20 carbon atoms; wherein

R may have in the bond thereof one or more intervening moieties selected from the group consisting of an oxygen atom, a sulfur atom, and a carbonyl group.

(3) An ether compound represented by the following formula (3),

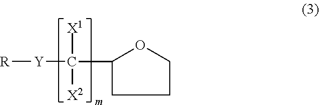

wherein

m represents an integer of 0 to 2,

Y represents any one selected from the group consisting of —O—, —S—, —C(═O)—O—, and —O—C(═O)—,

X¹ and X² each independently represent a hydrogen atom or a fluorine atom, and

R represents an aliphatic hydrocarbon group having 1 to 20 carbon atoms and substituted with one or more fluorine atoms, with a proviso that, when m is 0, R has 3 to 20 carbon atoms; wherein

R may have in the bond thereof one or more intervening moieties selected from the group consisting of an oxygen atom, a sulfur atom, and a carbonyl group.

(4) An electrolyte solution composition for a non-aqueous battery, comprising an organic solvent, an electrolyte dissolved in the organic solvent, and the ether compound according to any one of (1) to (3).

(5) A binder composition for a non-aqueous battery electrode, comprising an acrylic-based polymer and the ether compound according to any one of (1) to (3).

(6) A slurry composition for a non-aqueous battery electrode, containing an electrode active material and the binder composition for a non-aqueous battery electrode according to (5).

(7) An electrode for a non-aqueous battery, comprising a current collector and an electrode active material layer provided on a surface of the current collector,

wherein the electrode active material layer is obtained by applying and drying the slurry composition for a non-aqueous battery electrode according to (6).

(8) A non-aqueous battery, comprising a positive electrode, a negative electrode, and a non-aqueous electrolyte solution,

wherein the non-aqueous electrolyte solution is the electrolyte solution composition for a non-aqueous battery according to (4).

(9) A non-aqueous battery, comprising a positive electrode, a negative electrode, and a non-aqueous electrolyte solution,

wherein one or both of the positive electrode and the negative electrode is the electrode for a non-aqueous battery according to (7).

### Advantageous Effects of Invention

The ether compound of the present invention is a novel compound which has not conventionally existed.

By applying the electrolyte solution composition for a non-aqueous battery, the binder composition for a non-aqueous battery electrode, the slurry composition for a non-aqueous battery electrode, and the electrode for a non-aqueous battery of the present invention, to a non-aqueous battery, it is possible to realize a non-aqueous battery having high discharge capacity and excellent stability of charge-discharge cycle at high temperatures.

The non-aqueous battery of the present invention has a high discharge capacity and a stable charge-discharge cycle at high temperatures.

### DESCRIPTION OF EMBODIMENTS

The present invention will be described in detail by showing embodiments, examples, and the like. However the present invention is not limited to the following embodiments, examples, and the like, and may be arbitrary modified for implementation within a range without departing from the claims of the present invention, and its equivalents.

[1. Ether Compound of the Invention]

The ether compound of the present invention is a compound having a molecular structure represented by the following formula (1):

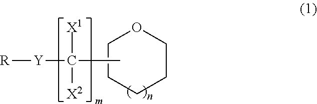

In the formula (1), n represents 0 or 1. It is preferable that n is 0 since thereby, when the ether compound of the present invention is applied to a non aqueous battery, it is expected that a favorable stable protective film is formed in the mechanism which will be described later so that the charge-discharge cycle at high temperatures is stabilized. That is, it is preferable that the ether ring the ether compound of the present invention has is a five-membered ring.

In the formula (1), m represents an integer equal to or greater than 0 and equal to or smaller than 2. It is preferable that m is 1 since thereby, when the ether compound of the present invention is applied to a non-aqueous battery, the effects can be obtained more reliably and the synthesis can be performed at low cost.

In the formula (1), Y represents any divalent linking group selected from the group consisting of —O—, S—, —C(═O)—O— and —C(═O)—. In this enumeration, —C(═O)—O— and —O—C(═O)— are listed in a distinguished manner. This intends to clarify that, when Y is an ester bond, the orientation of the bond may be either one of them. Among these divalent linking groups, —O— is preferable. When the ether compound of the present invention is applied to a non-aqueous battery, the compound having these linking groups in the bond does not inhibit insertion and extraction of lithium ions in the mechanism which will be described later. Therefore a high discharge capacity can be realized.

In the formula (1), X¹ and X² each independently represent a hydrogen atom or a fluorine atom. If m is 2, two X¹'s and two X²'s exist in the molecule represented by the formula (1). In this case, the two X¹'s may be the same or different, and the two X²'s may be the same or different. Specifically, it is preferable that X¹ and X² are hydrogen atoms since thereby, when the ether compound of the present invention is applied to a non-aqueous battery, the effects can be obtained more reliably.

In the formula (1), R represents an aliphatic hydrocarbon group substituted with a fluorine atom. The aliphatic hydrocarbon group may be an aliphatic saturated hydrocarbon group or an aliphatic unsaturated hydrocarbon group. When the aliphatic hydrocarbon group is an aliphatic unsaturated hydrocarbon group, the unsaturated bond may be a double bond or a triple bond. The number of unsaturated bonds in the group may be one, or two or greater. In particular, the aliphatic hydrocarbon group is preferably an aliphatic saturated hydrocarbon group since thereby, when the ether compound of the present invention is applied to a non-aqueous battery, the effects can be obtained more reliably.

The aliphatic hydrocarbon group in R may be a linear aliphatic hydrocarbon group without a branch in the carbon chain or a branched chain aliphatic hydrocarbon group with a branch in the carbon chain. In particular, the aliphatic hydrocarbon group is preferably a linear aliphatic hydrocarbon group since thereby, when the ether compound of the present invention is applied to a non-aqueous battery, the effects can be obtained more reliably, and the synthesis can be performed at low cost.

The aliphatic hydrocarbon group in R may have in the bond thereof one or more intervening groups-Selected from the group consisting of an oxygen atom, a sulfur atom, and a carbonyl group. Two or more of these oxygen atom, sulfur atom, and carbonyl group may be combined. For example, an oxygen atom and a carbonyl group may be combined to form an ester bond (—COO—) which may present in the middle of the bond. Furthermore, the number of the oxygen atom, sulfur atom, or carbonyl group that is present in the middle of the bond may be one, or two or greater. The oxygen atom, sulfur atom, or carbonyl group may be present in the middle of the carbon-carbon bond of the aliphatic hydrocarbon group, or may be present in the middle of the bond at the terminal of the aliphatic hydrocarbon group (that is, the bond between R and Y in the formula (1)). Especially, it is preferable that the oxygen atom, sulfur atom, or carbonyl group is present in the middle of the carbon-carbon bond.

The position of a fluorine atom in R is not limited. However, it is preferable that the fluorine atom is attached to the carbon atom located at the terminal of the aliphatic hydrocarbon group of R. By having a large number of fluorine atoms at the terminal of the group “—(CX¹X²)ₘ—Y—R” which is bonded to the ether ring, the polarity of the stable protective film that is generated in the mechanism which will be described later can be enhanced when the ether compound of the present invention is applied to a non-aqueous battery. Accordingly, it is possible to effectively inhibit reduction in the discharge capacity caused by the stable protective film. Furthermore, since it is preferable that many fluorine atoms are bonded to the carbon atom located at the terminal of the aliphatic hydrocarbon group of R, the number of carbon atoms which are bonded to the carbon atom located at the terminal of the aliphatic hydrocarbon group of R is usually equal to or greater than 1, preferably equal to or greater than 2, and more preferably 3.

The number of fluorine atoms that R has may be one, or two or greater. Since it is preferable that many fluorine atoms are bonded to the carbon atom located at the terminal of the aliphatic hydrocarbon group in R as described above, it is preferable that the number of fluorine atoms is 3 or greater. The number of fluorine atoms is preferably equal to or smaller than 15, and more preferably equal to dr smaller than 11. By having the fluorine atoms the number of which falls within the aforementioned range, an excellent charge-discharge cycle can be obtained.

The number of carbon atoms of R is 1 to 20. However, when m is 0, the number of carbon atoms in R is usually 3 to 20. Especially, the number of carbon atoms in R is preferably equal to or greater than 2, and preferably equal to or smaller than 10, more preferably equal to or smaller than 8 since thereby, when the ether compound of the present invention is applied to a non-aqueous battery, it is expected that a favorable stable protective film is formed in the mechanism which will be described later so that the charge-discharge cycle at high temperatures is stabilized. Although the number of carbon atoms in R usually refers to the number of carbon atoms in the aliphatic hydrocarbon group in R, when an intervening carbonyl group is present in the middle of the bond of R, the number refers to the number of carbon atoms including the number of carbon atoms of the carbonyl group.

Among them, it is particularly preferable that R is a group represented by the following formula (4).

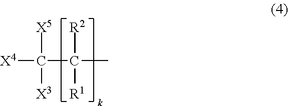

In the formula (4), k represents an integer of 0 to 19. However, when m is 0 in the formula (1), k represents an integer of 2 to 19. Especially, k is preferably equal to or smaller than 10, and more preferably equal to or smaller than 5.

In the formula (4), X³ to X⁵ represent a hydrogen atom or a fluorine atom. Especially, it is preferable that any one or more of X³ to X⁵ are fluorine atoms, and it is more preferable that all of X³ to X⁵ are fluorine atoms.

In the formula (4), each of R¹ and R² independently represents any one selected from the group consisting of a hydrogen atom, a fluorine atom, and an aliphatic saturated hydrocarbon group optionally having a fluorine atom as a substituent. Especially, as R¹ and R², a hydrogen atom or a fluorine atom is preferable since thereby, when the ether compound of the present invention is applied to a non-aqueous battery, the effects can be obtained more reliably. When both two or more R¹'s and two or more R²'s exist in the group represented by the formula (4), the two or more R¹'s may be the same or different, and the two or more R²'s may be also the same or different. The number of carbon atoms in R¹ and R² are determined such that the number of carbon atoms contained in the group represented by the formula (4) falls within a range of the number of carbon atoms in R of the formula (1).

Furthermore, in the formula (1), it is preferable that the group “—(CX¹X²)—Y—R” is bonded to the ether ring at the carbon atom of the ether ring that is bonded to the oxygen atom. That is, it is preferable that the ether compound of the present invention is represented by the following formula (2). In the formula (2), m, n, Y and R are the same as those in the formula (1).

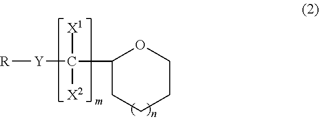

Furthermore, since it is preferable that n is 0 (zero) as described above, it is more preferable that the ether compound of the present invention is represented by the following formula (3). In the formula (3), m, Y, X², and R are the same as those in the formula (1).

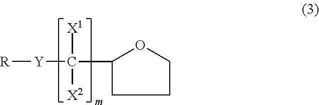

Examples of the ether compound of the present invention may include the following. However, the ether compound of the present invention is not limited to the examples listed below since the requirement for exhibiting the effects of the ether compound of the present invention is the presence of the structure in which the cyclic ether skeleton and the aliphatic hydrocarbon group containing a fluorine atom are bonded through the specific heteroatom-containing linking group.

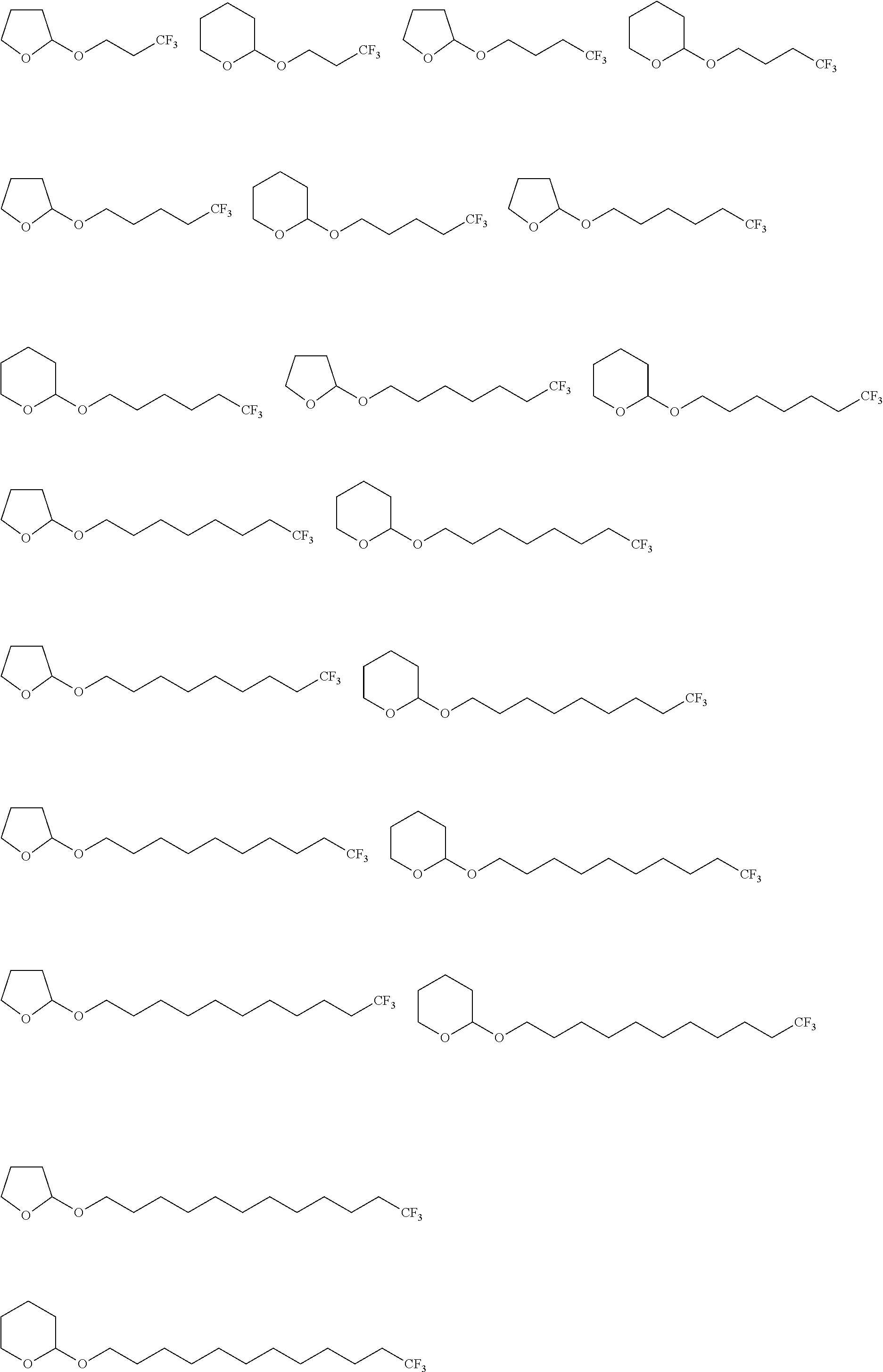

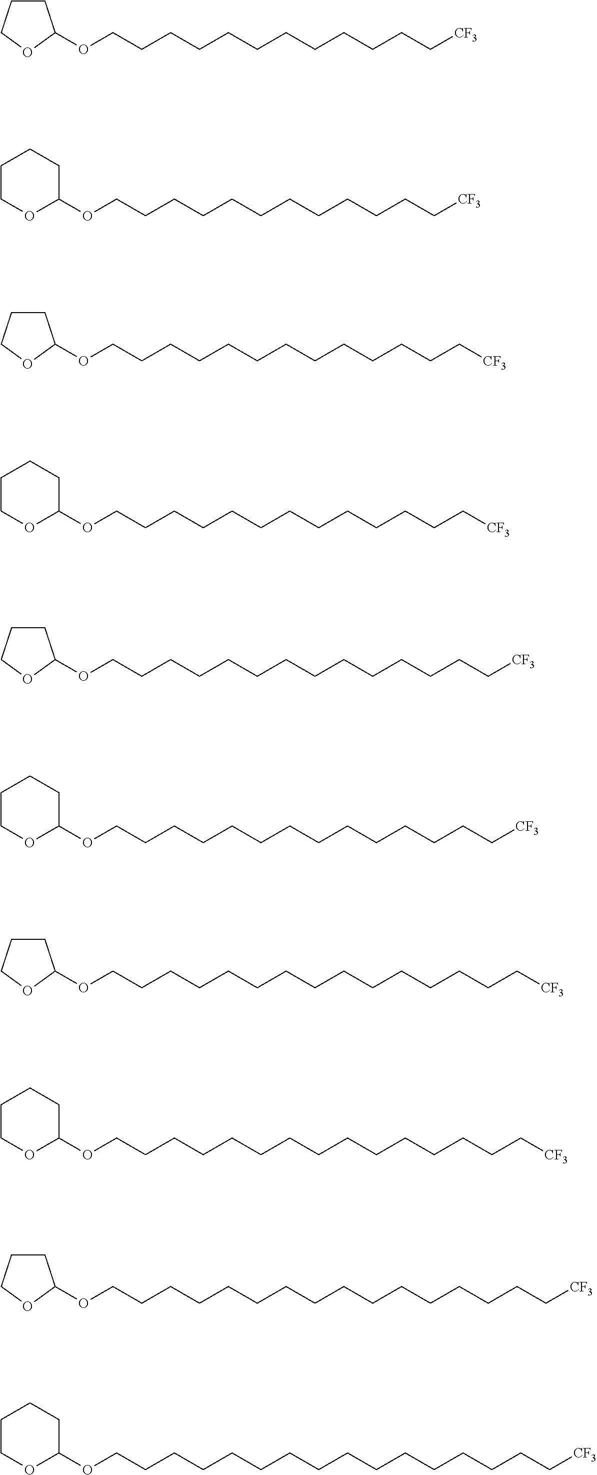

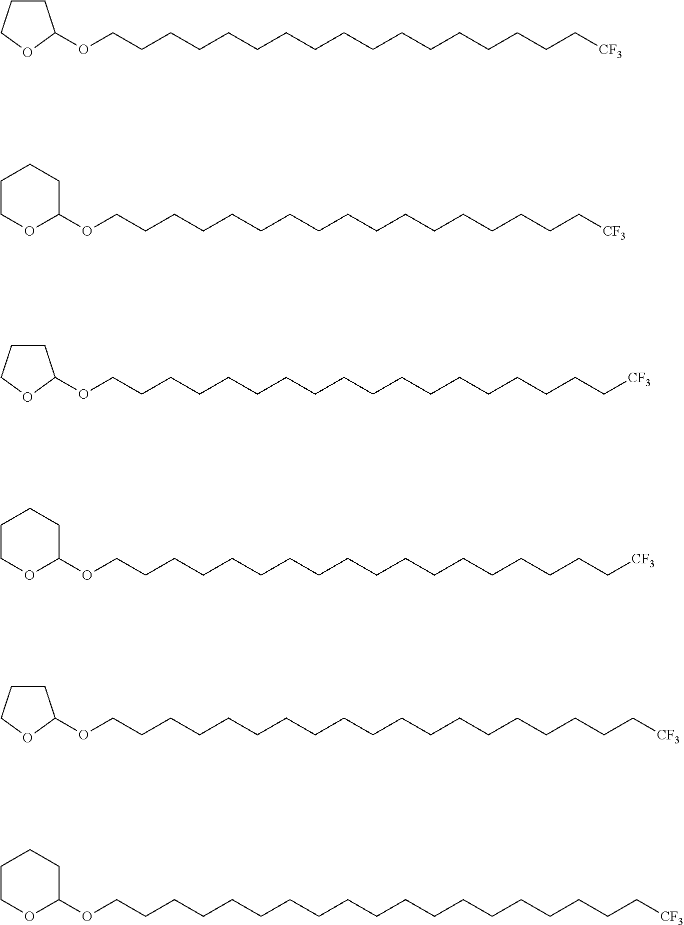 [SMILES: FC(F)(F)CCCCCCCCCCCCCCCCCOC1CCCO1 ; FC(F)(F)CCCCCCCCCCCCCCCCCOC1CCCCO1 ; FC(F)(F)CCCCCCCCCCCCCCCCCCOC1CCCO1 ; FC(F)(F)CCCCCCCCCCCCCCCCCCOC1CCCCO1 ; FC(F)(F)CCCCCCCCCCCCCCCCCCCOC1CCCO1 ; FC(F)(F)CCCCCCCCCCCCCCCCCCCOC1CCCCO1]

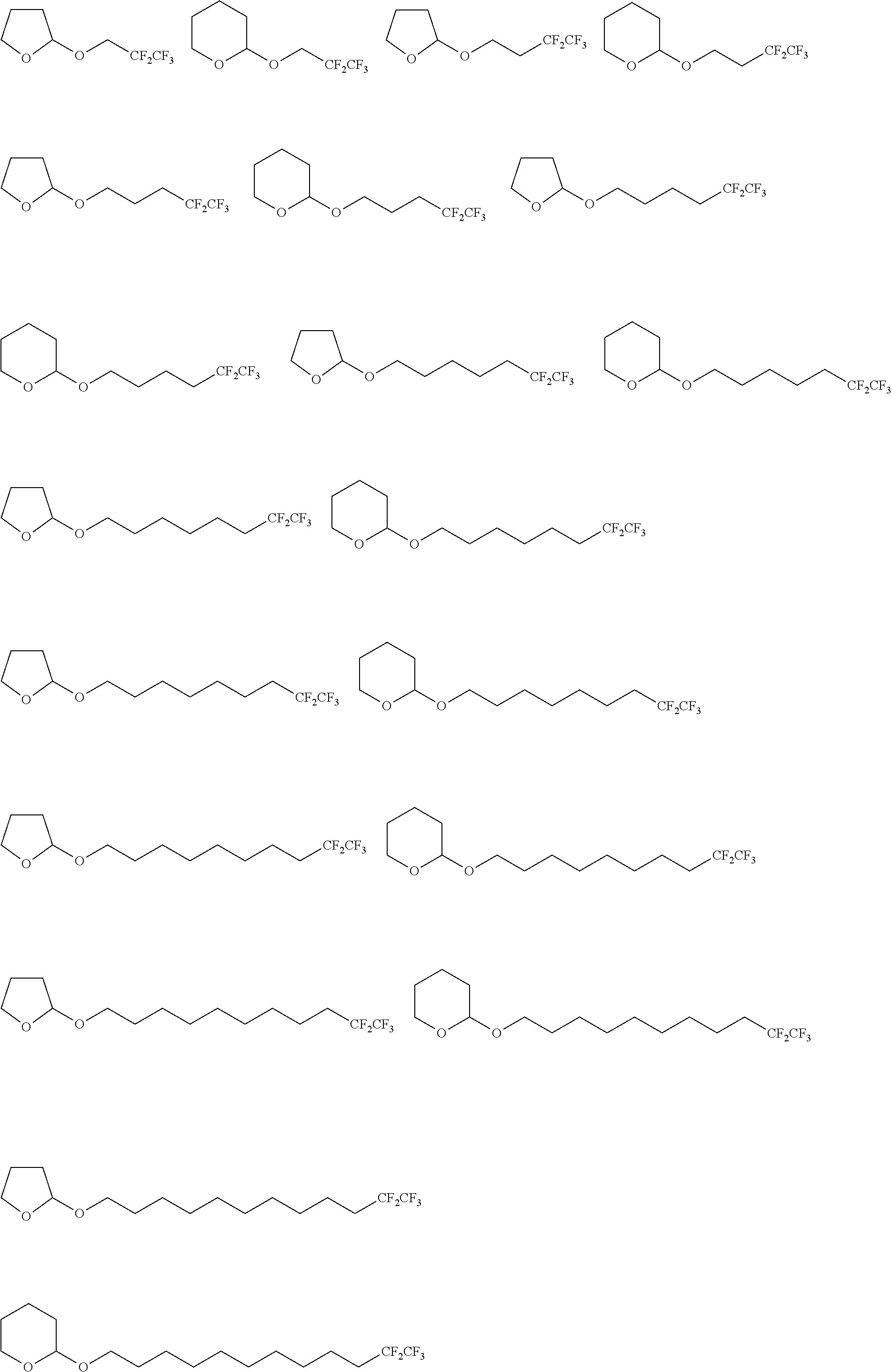

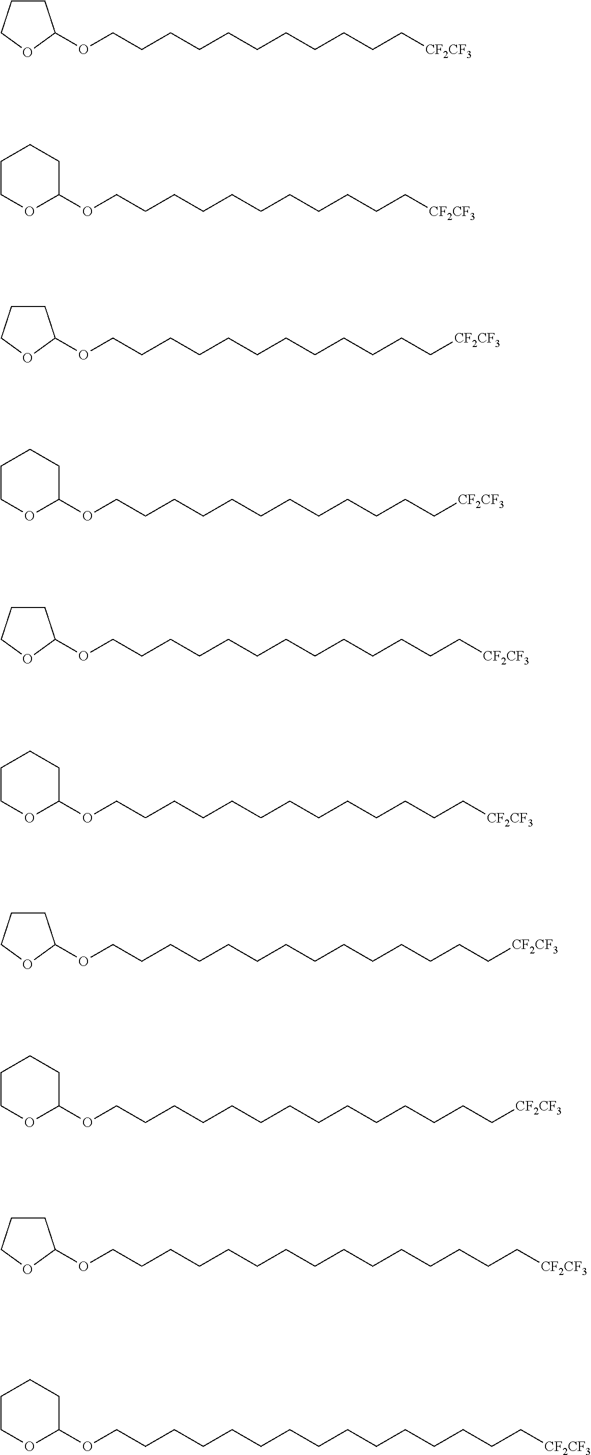

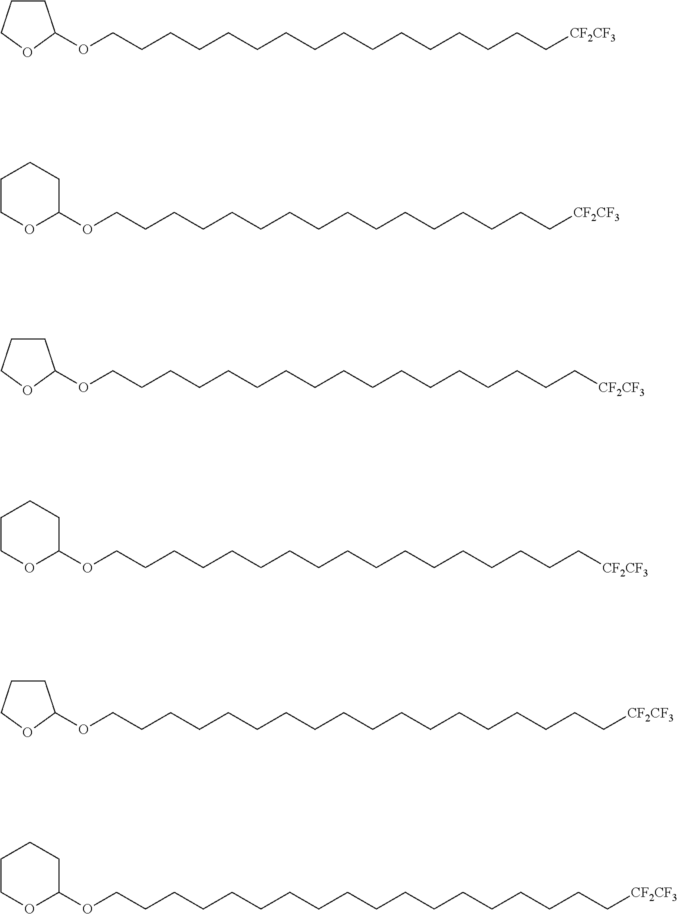

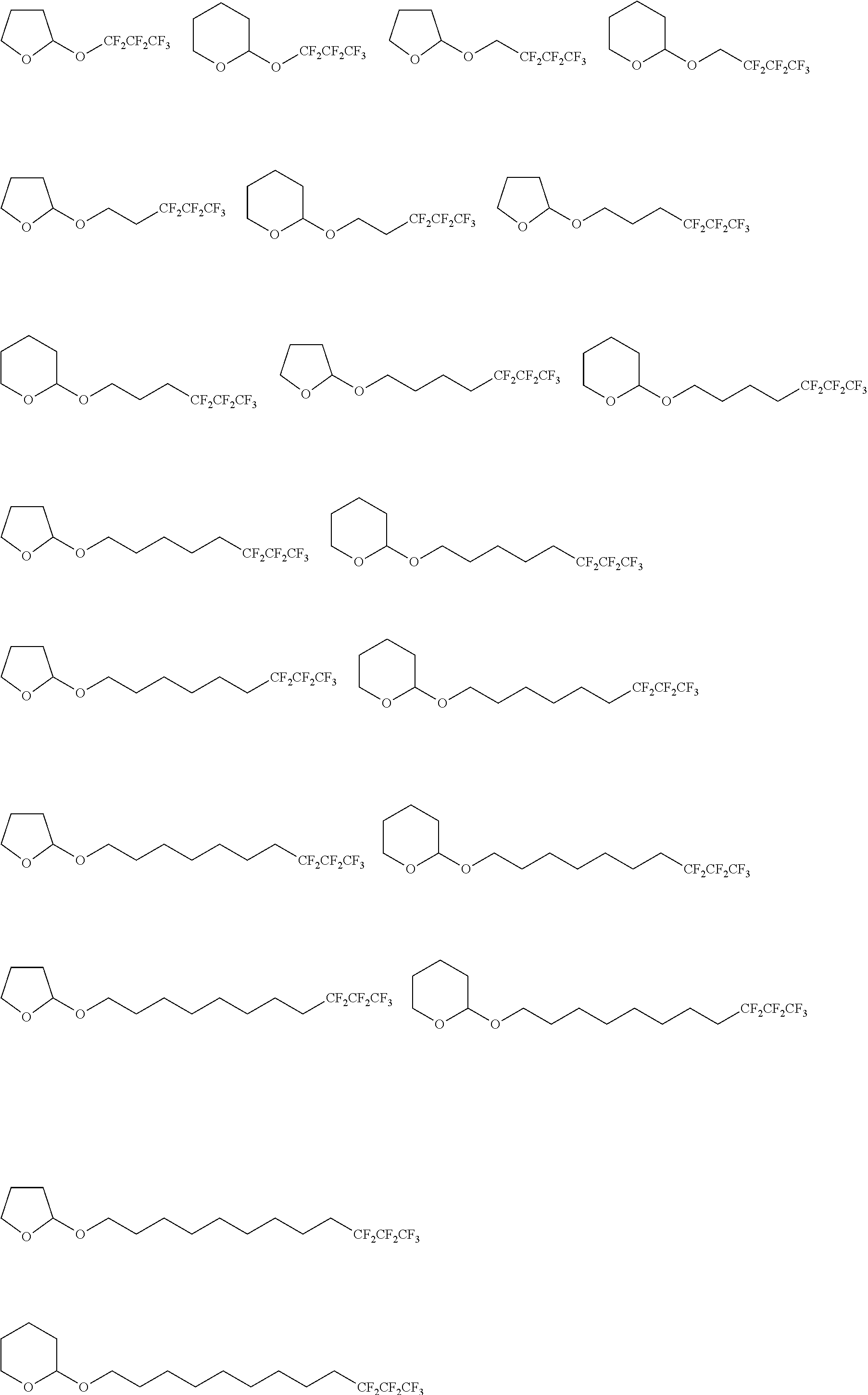

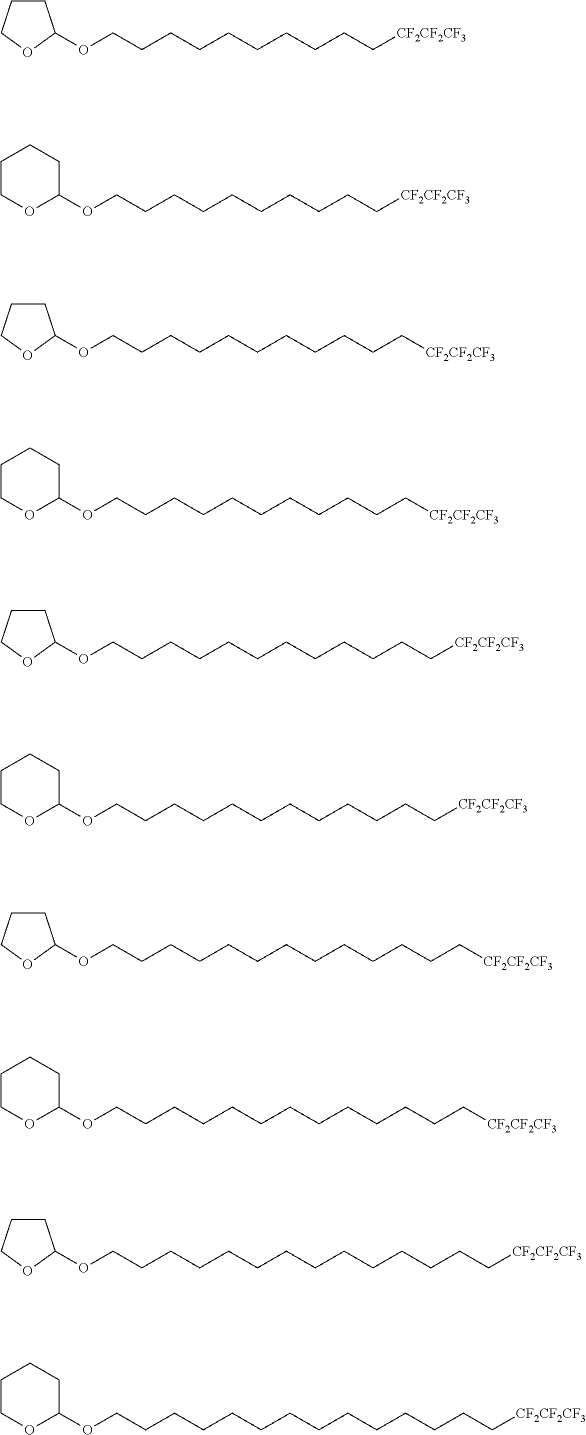

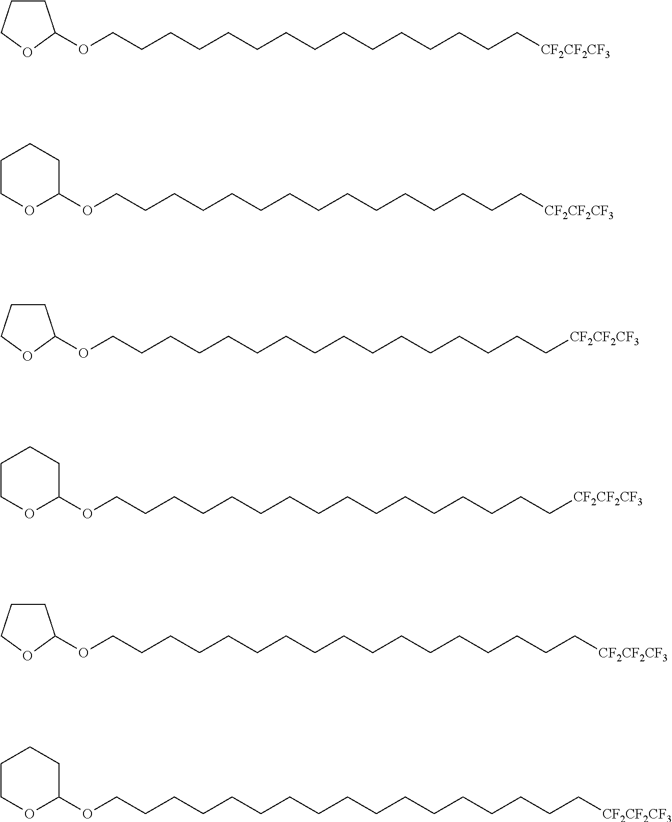

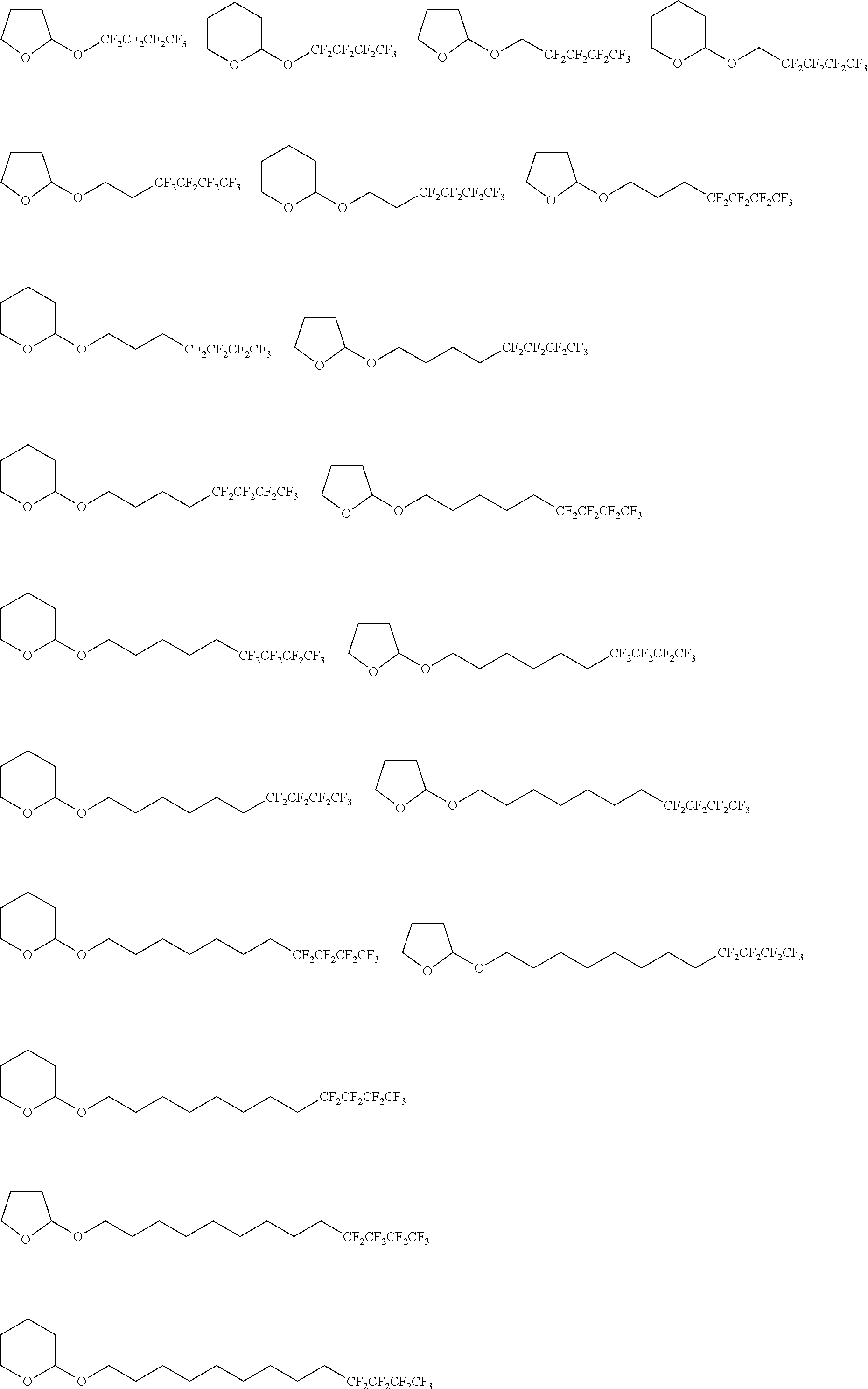

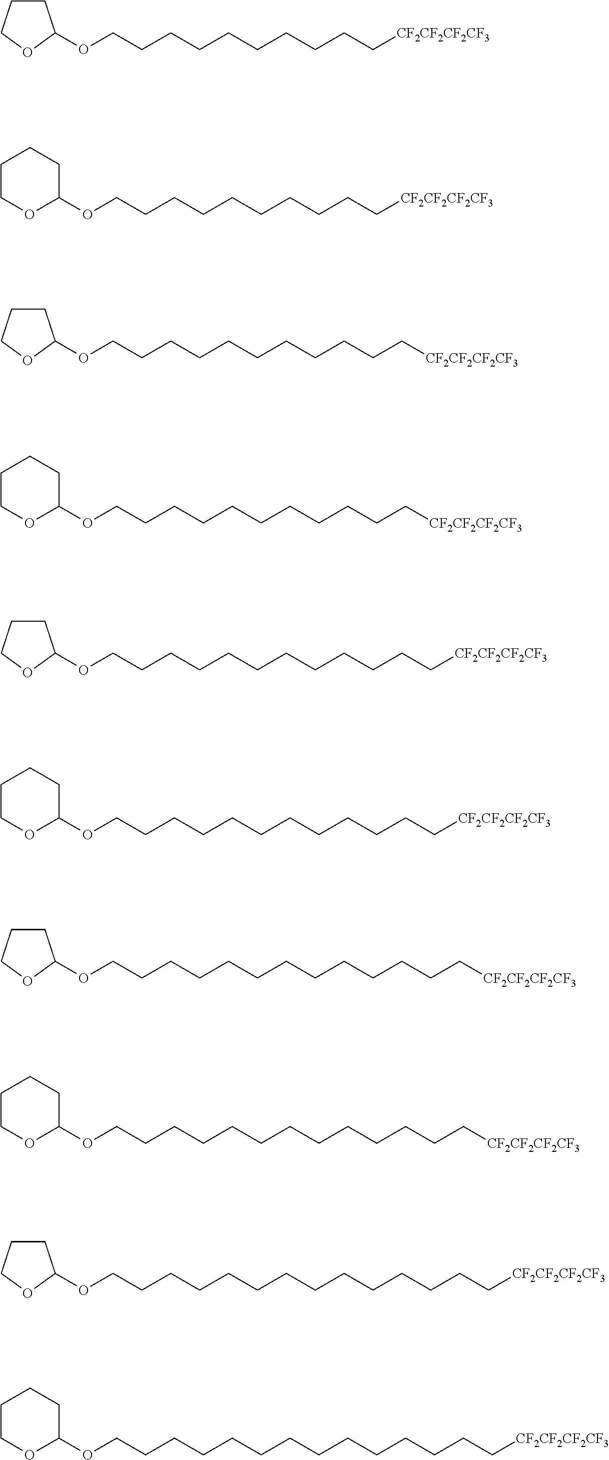

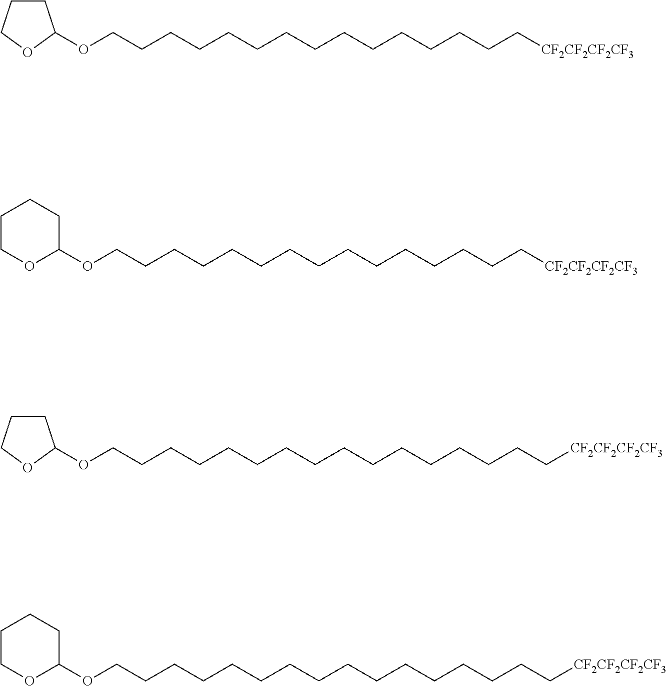

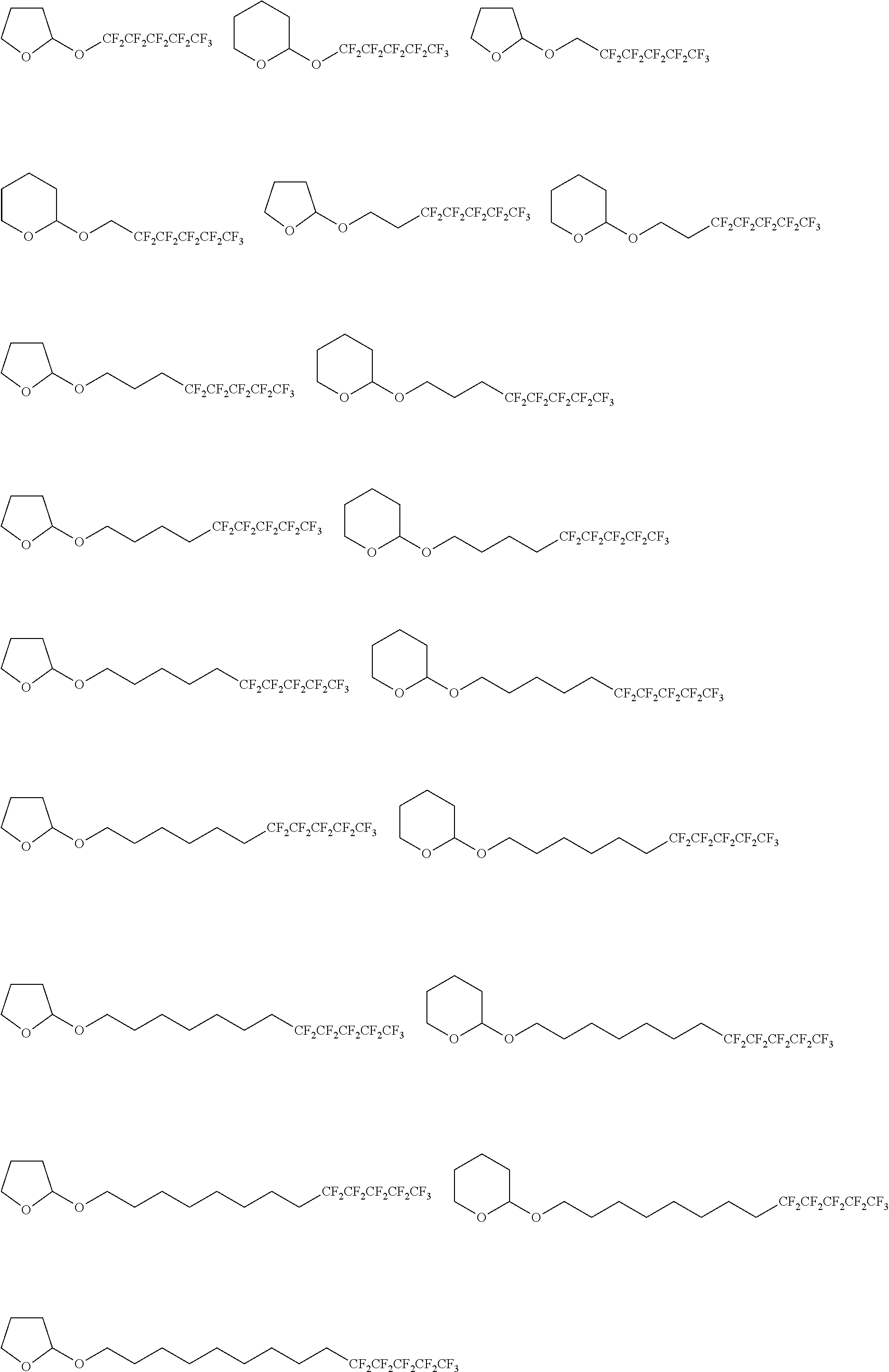

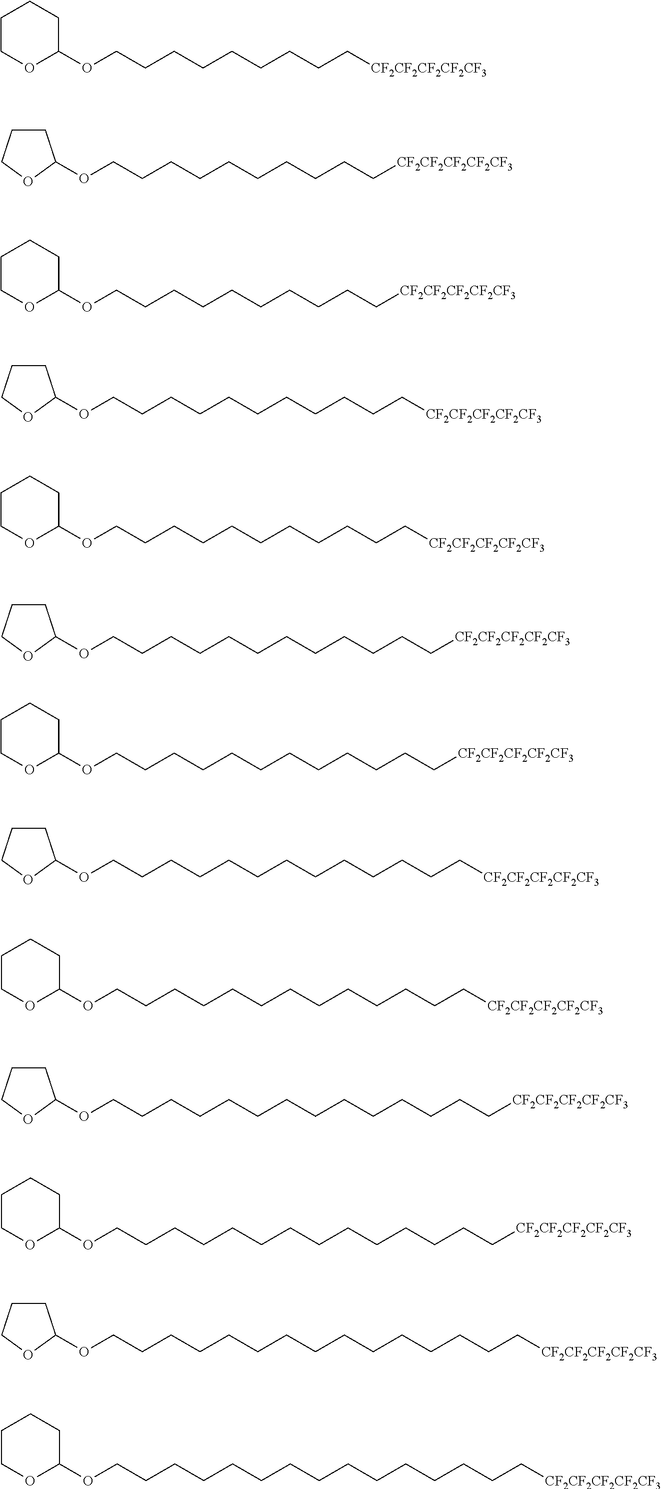

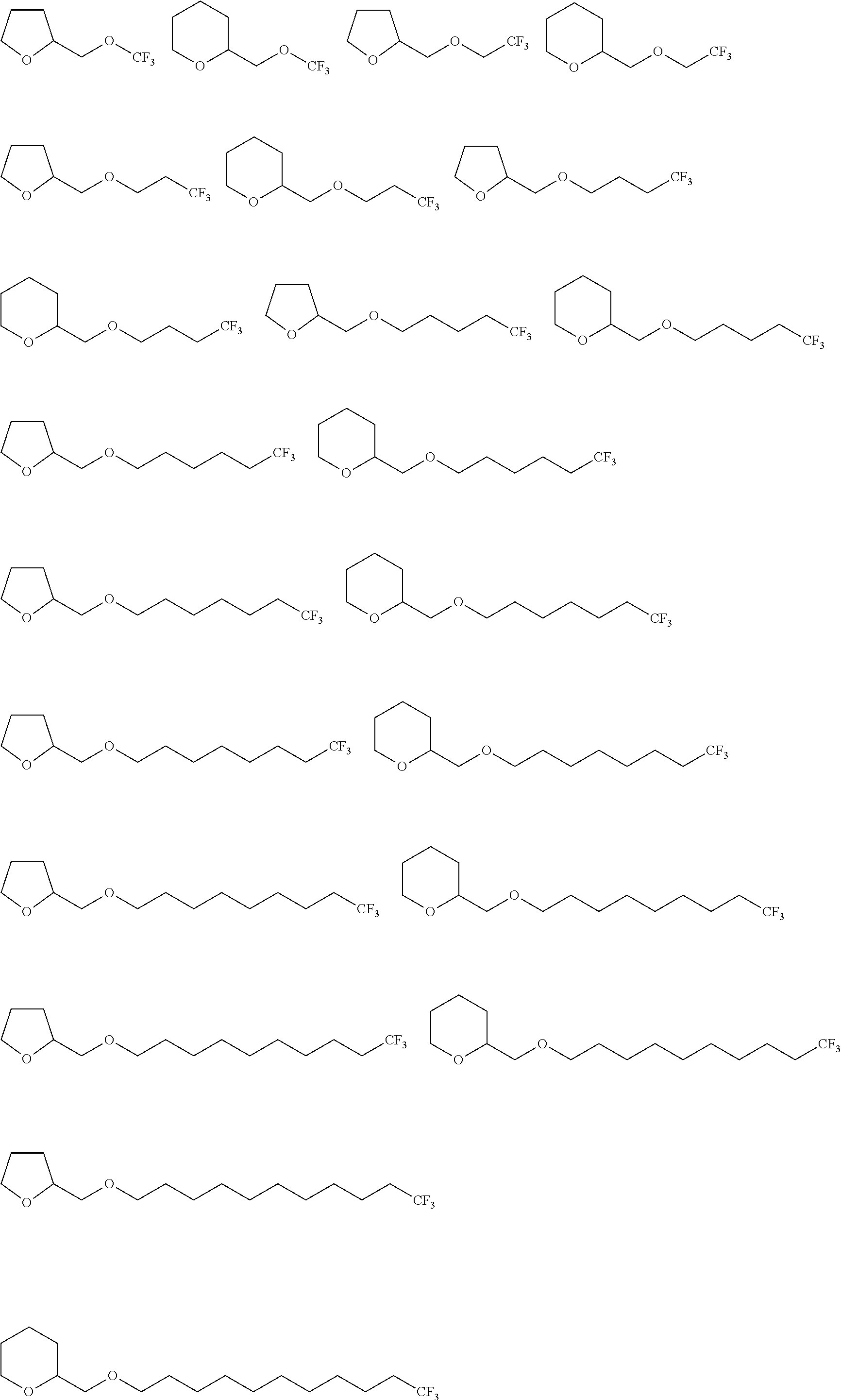

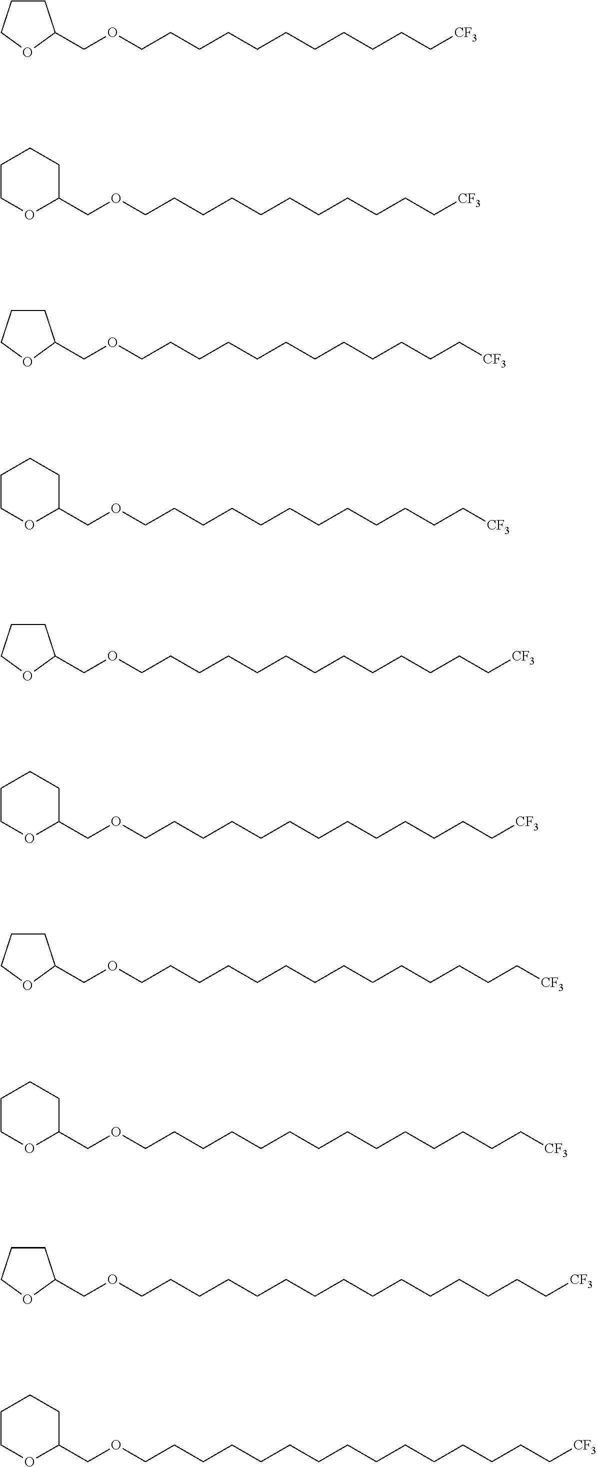

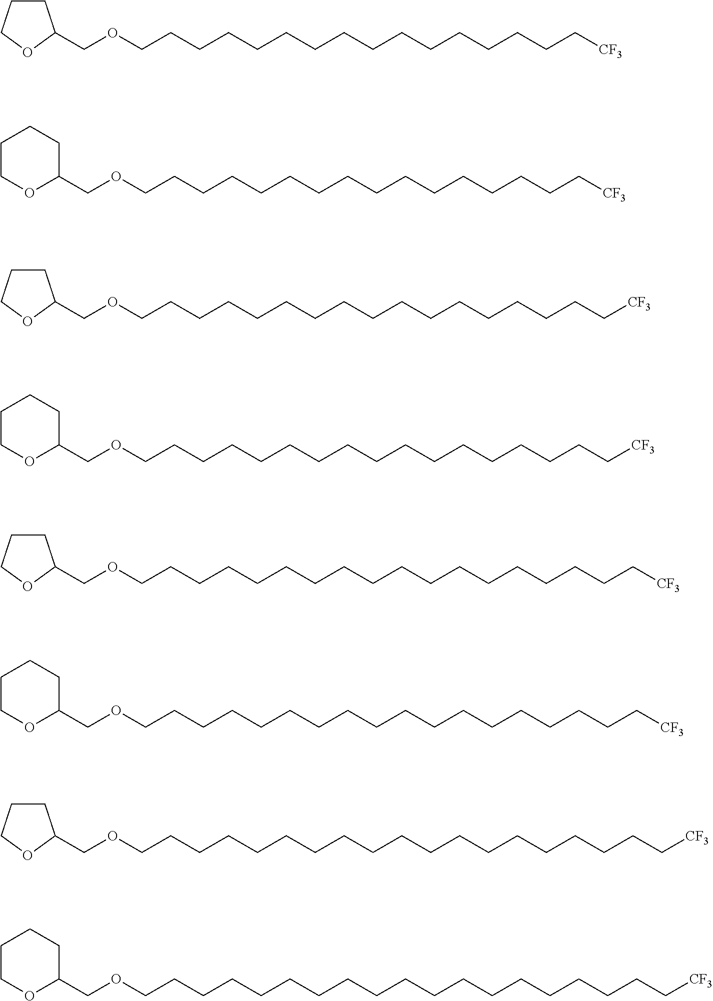

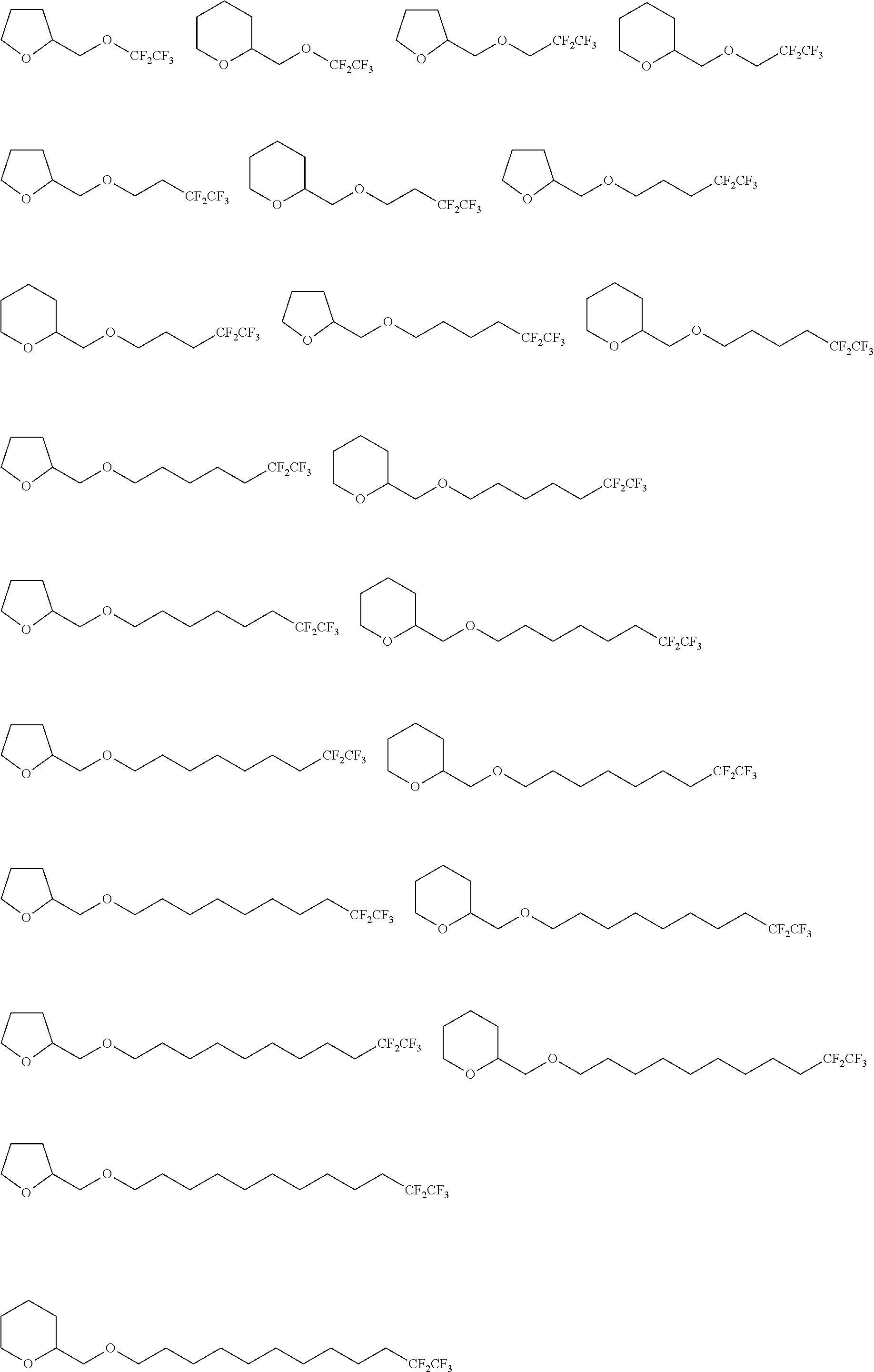

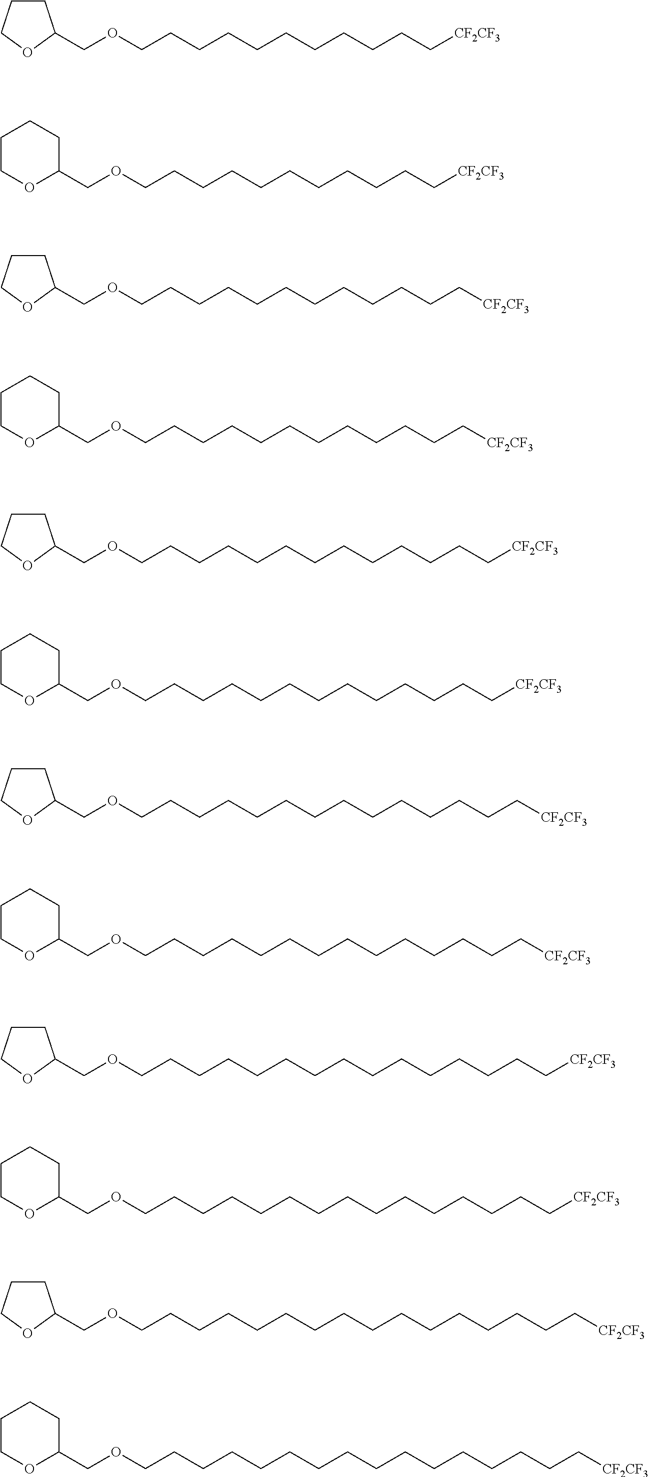

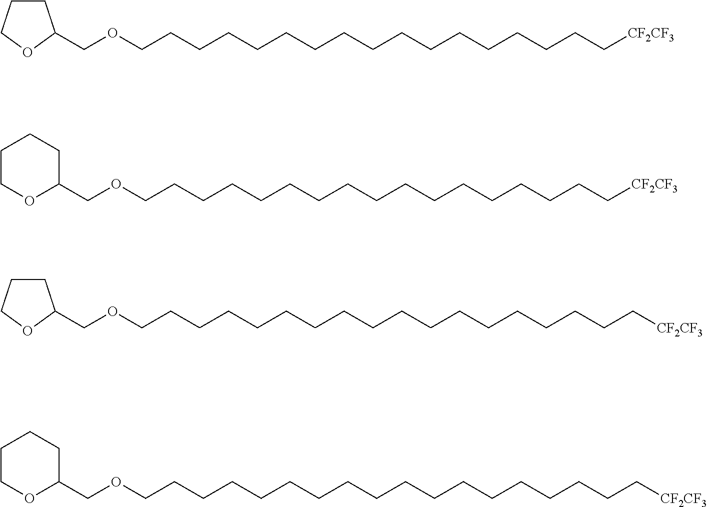

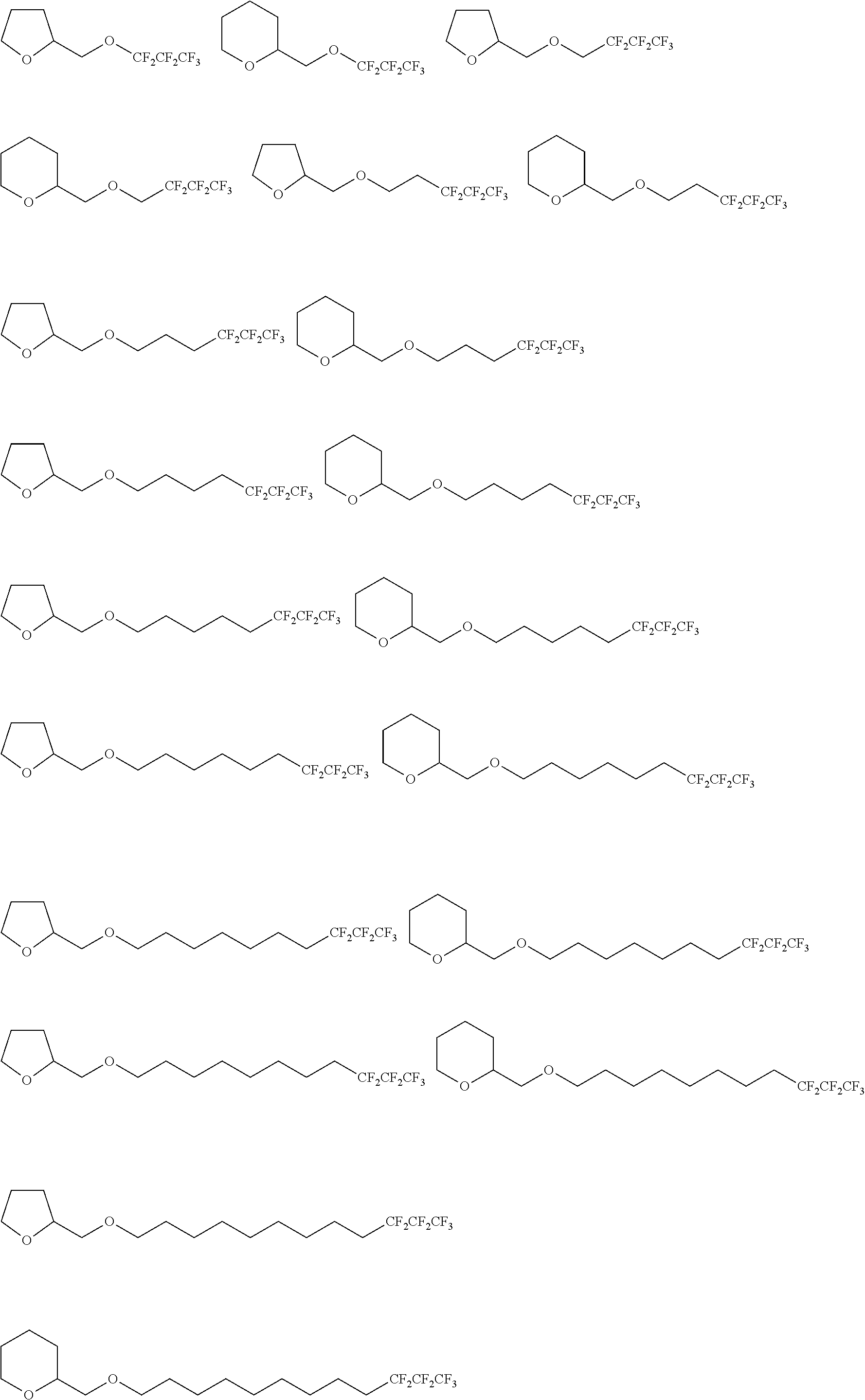

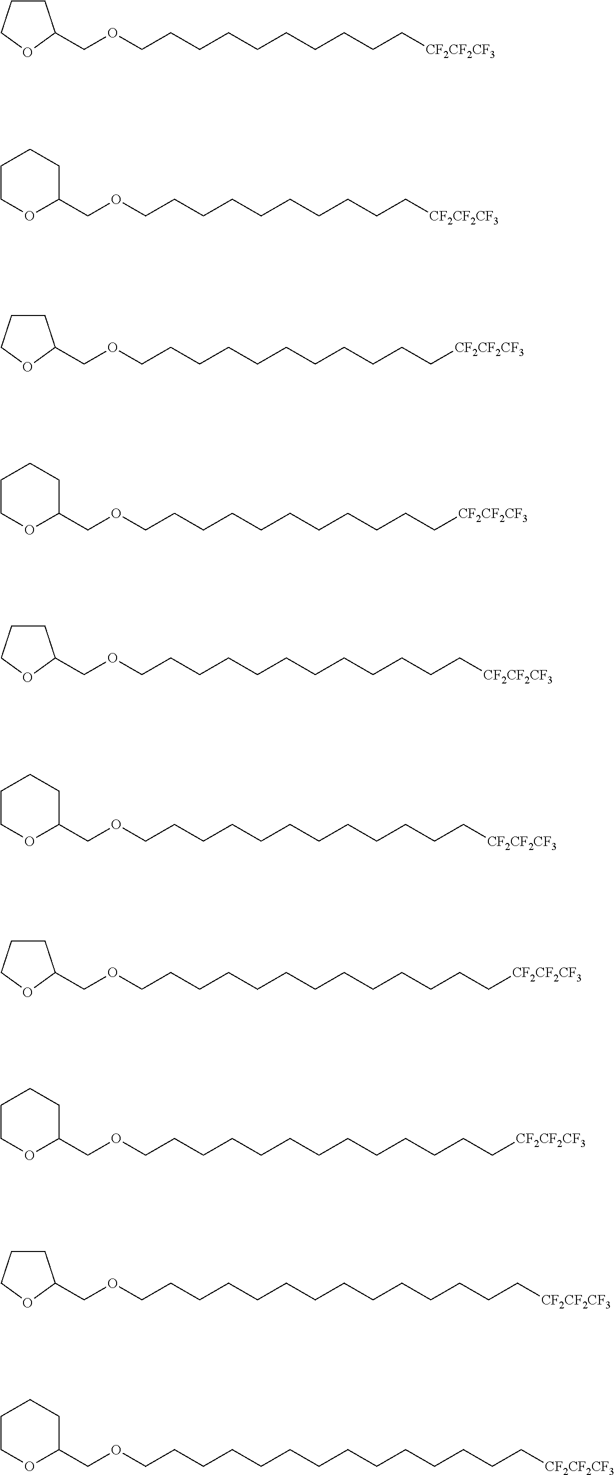

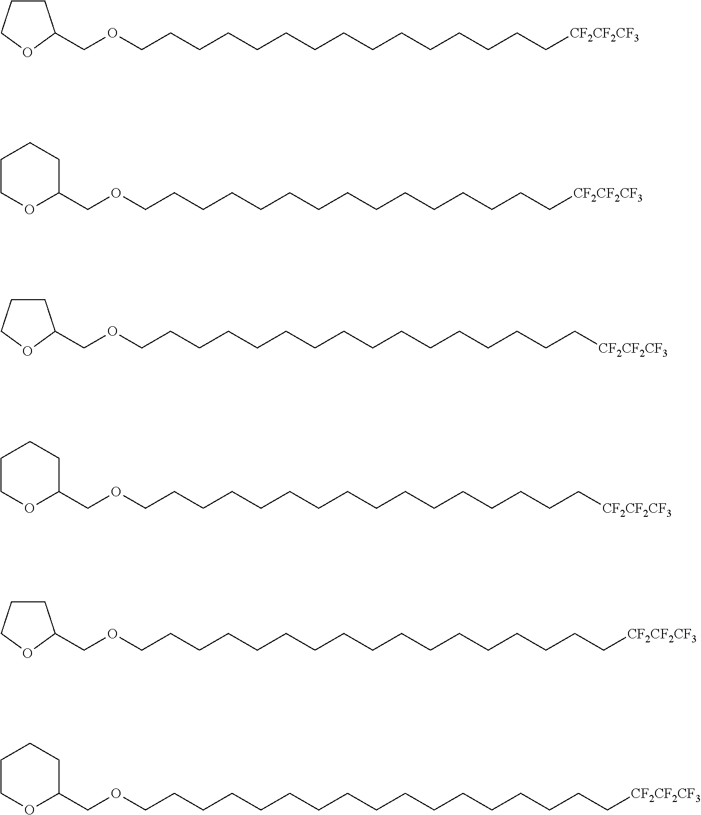

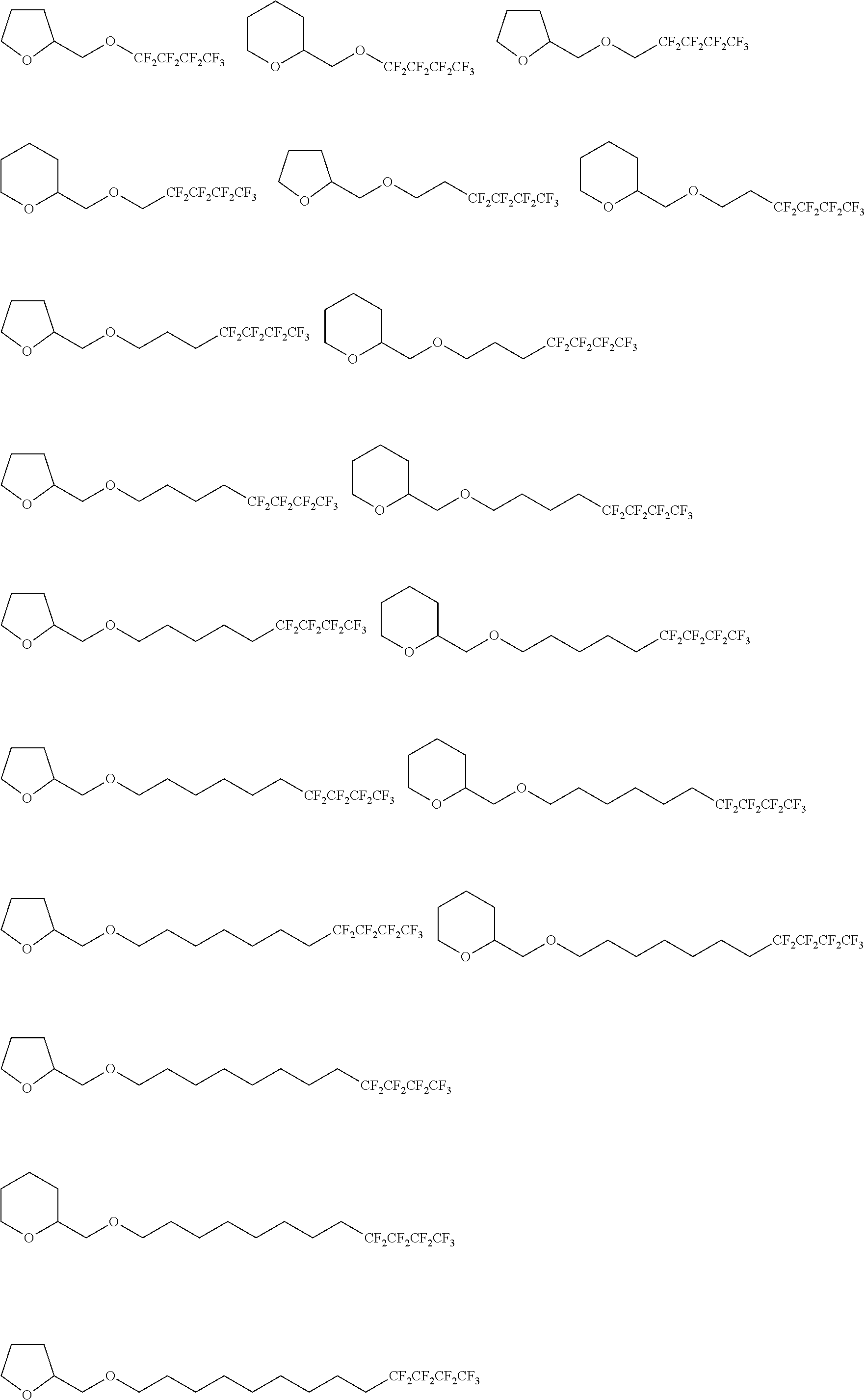

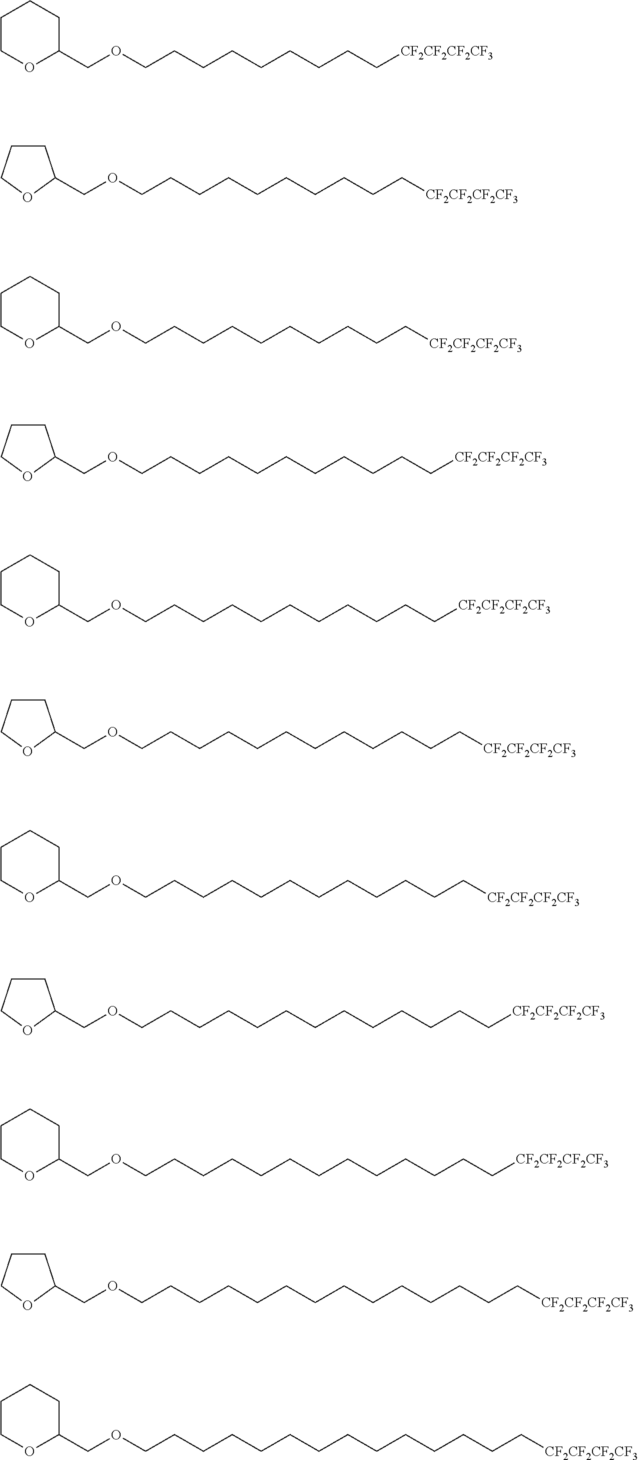

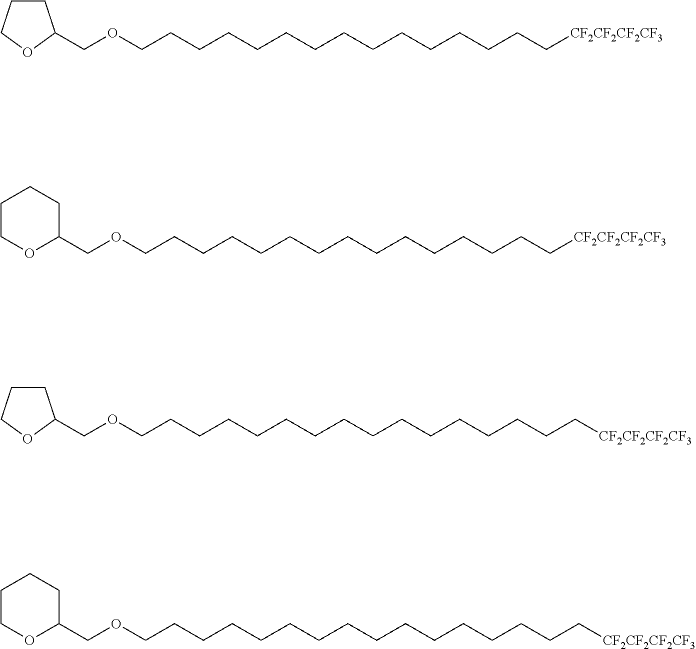

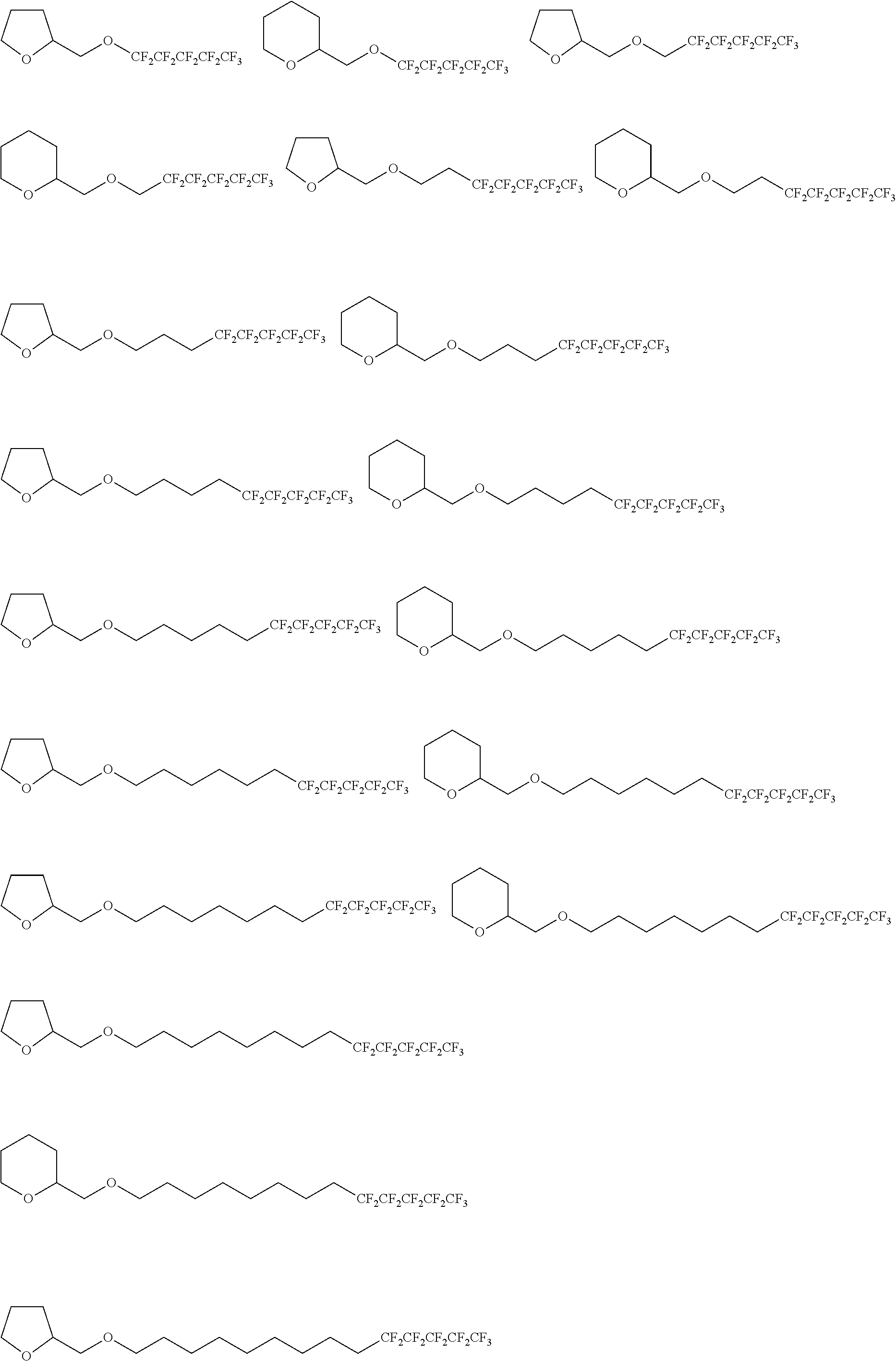

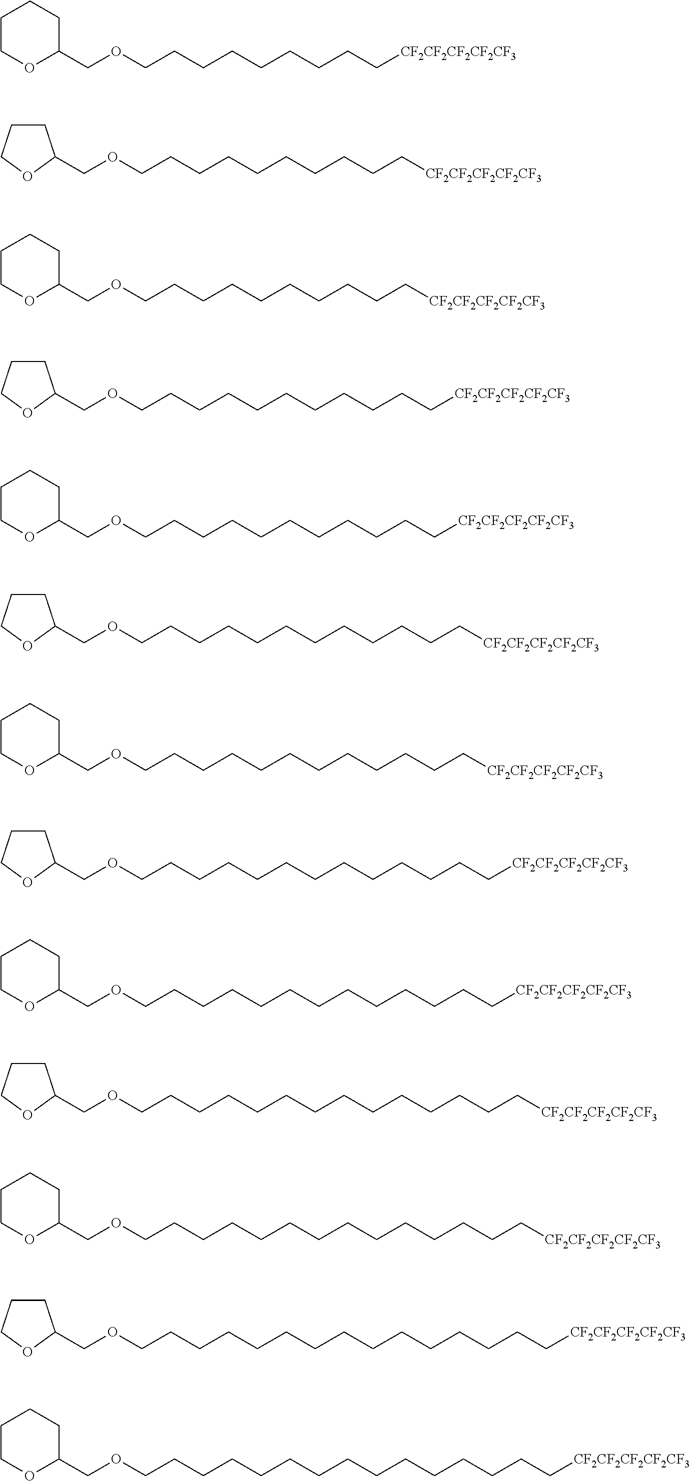

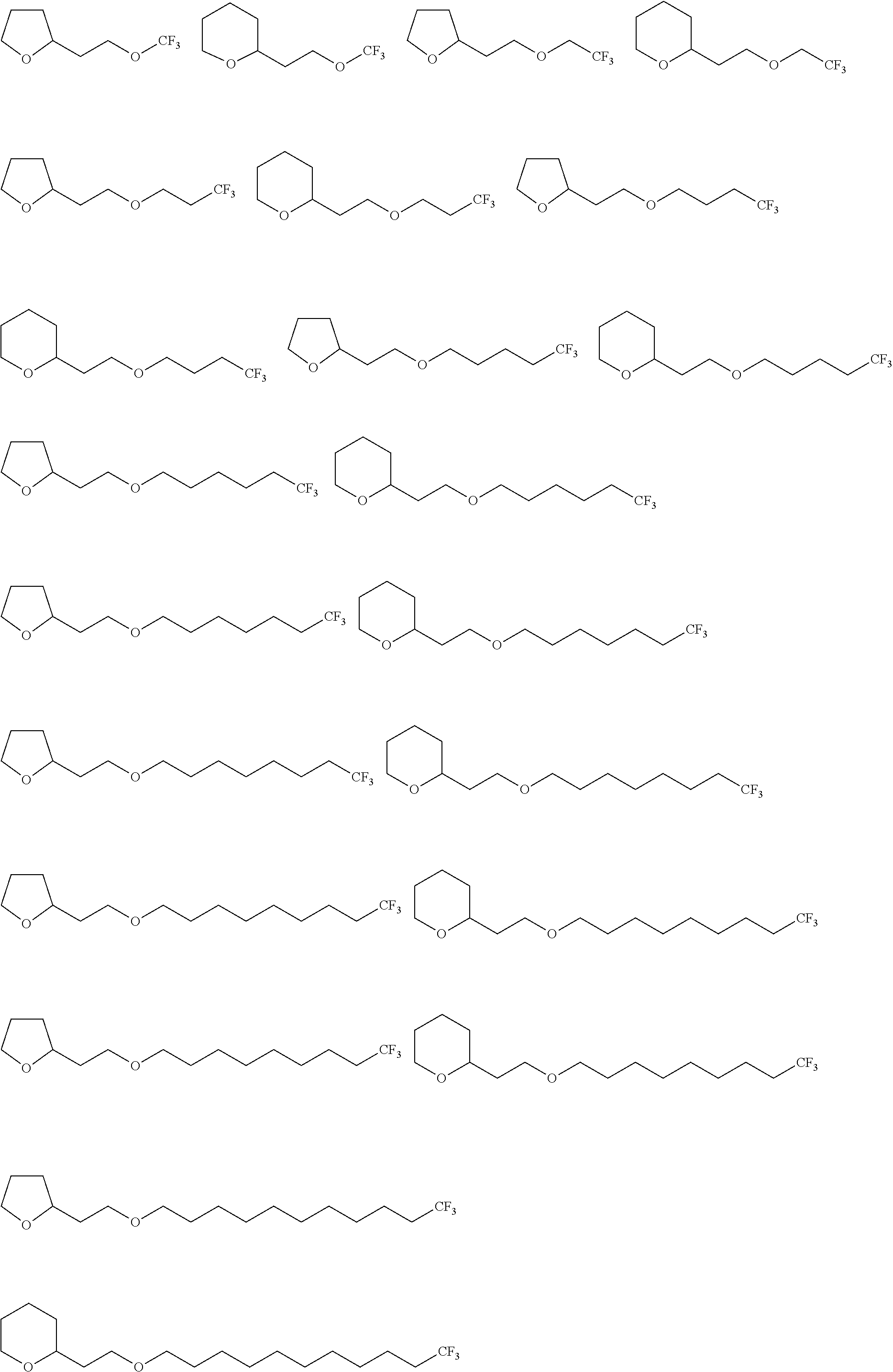

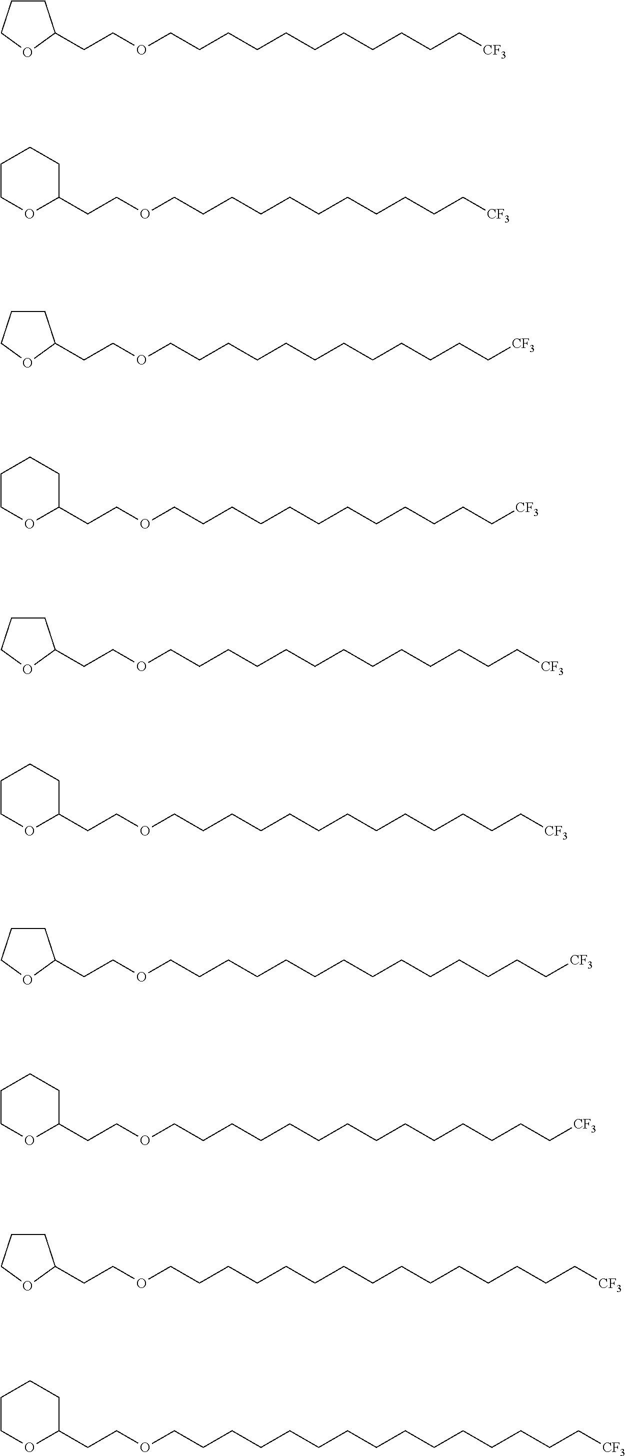

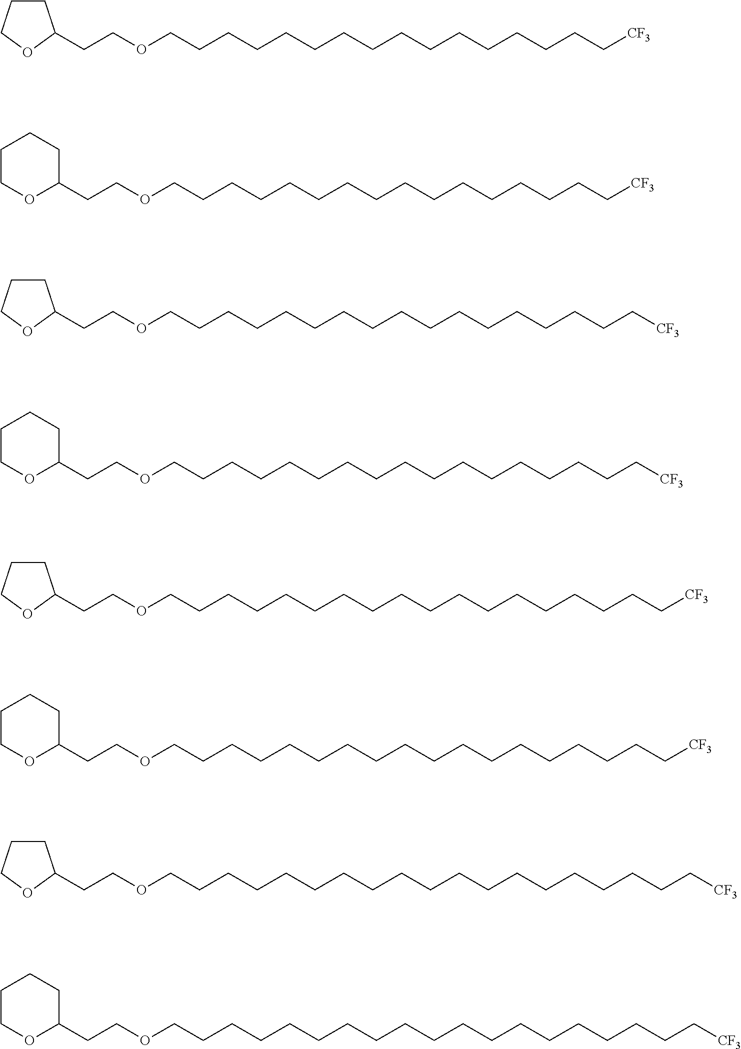

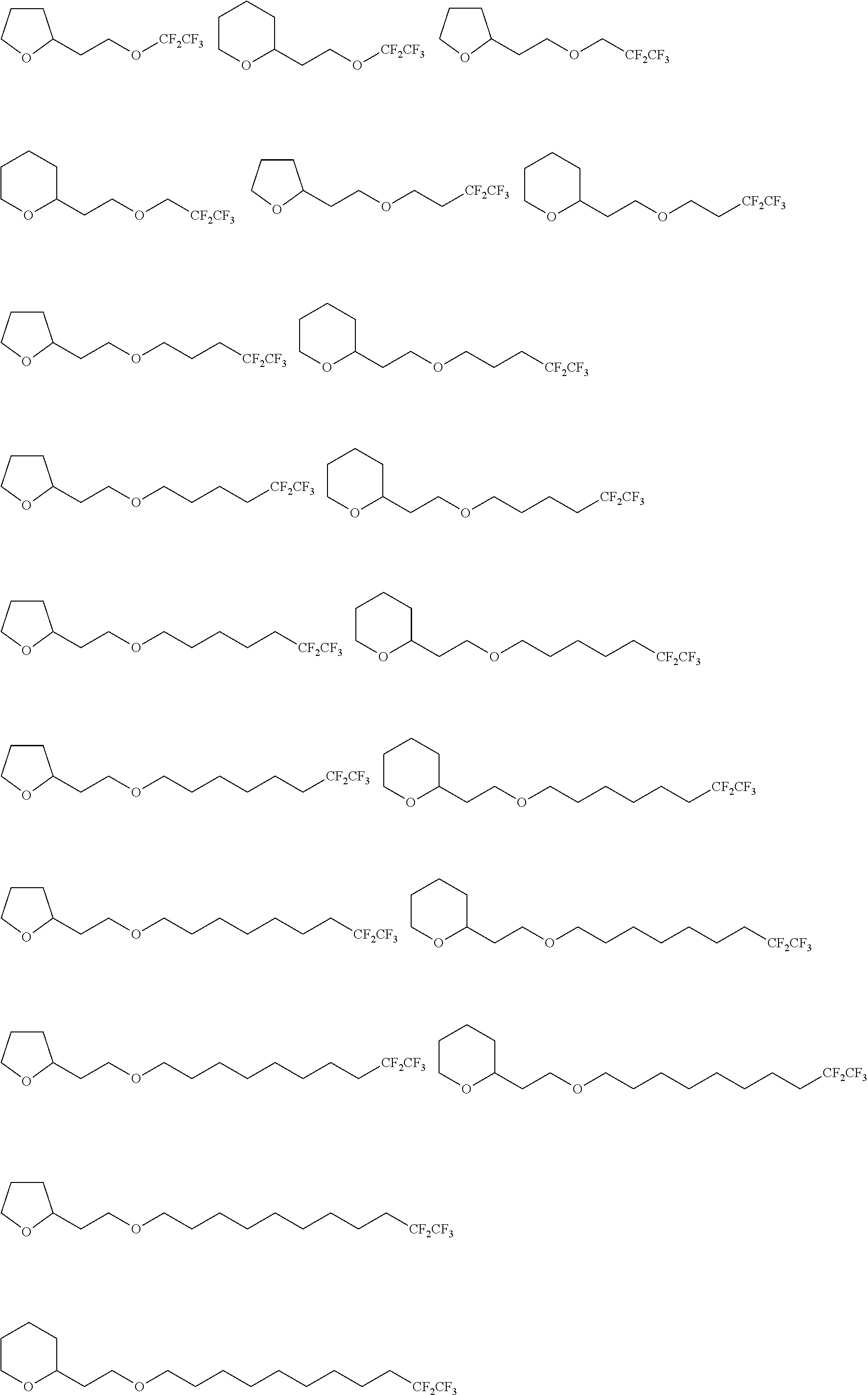

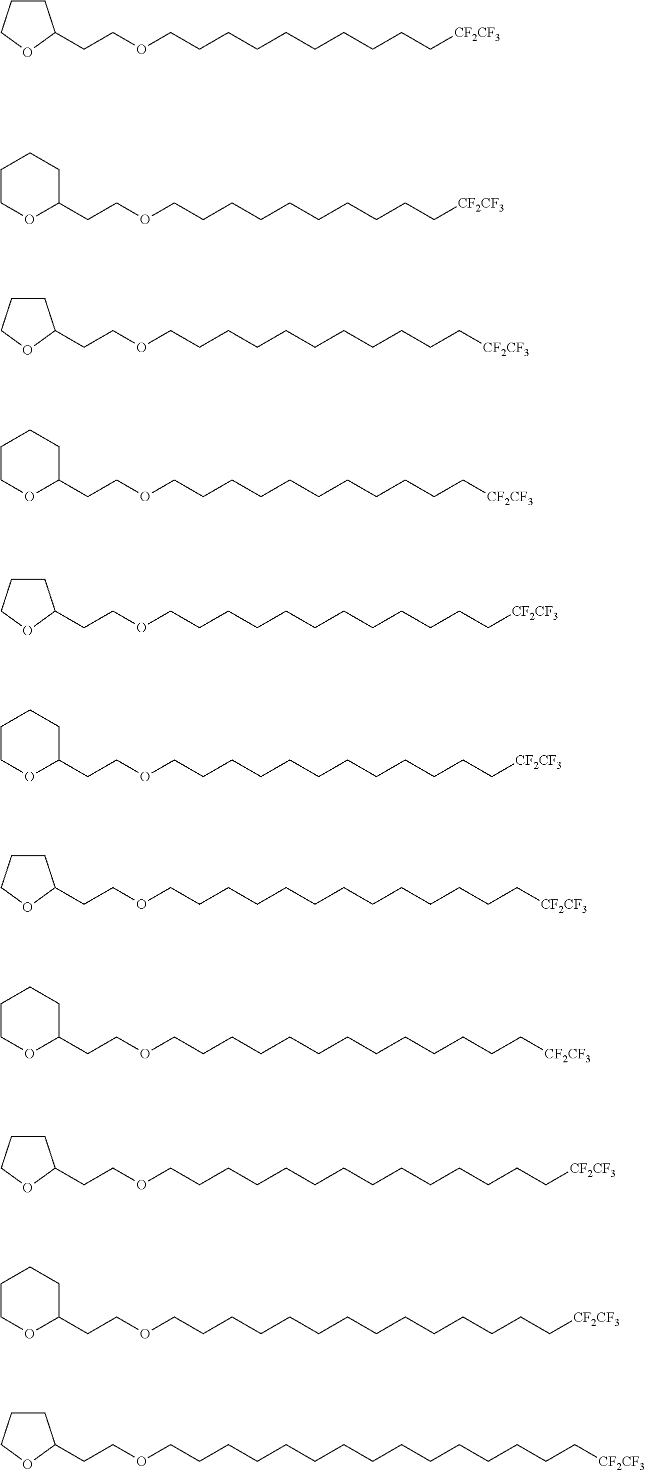

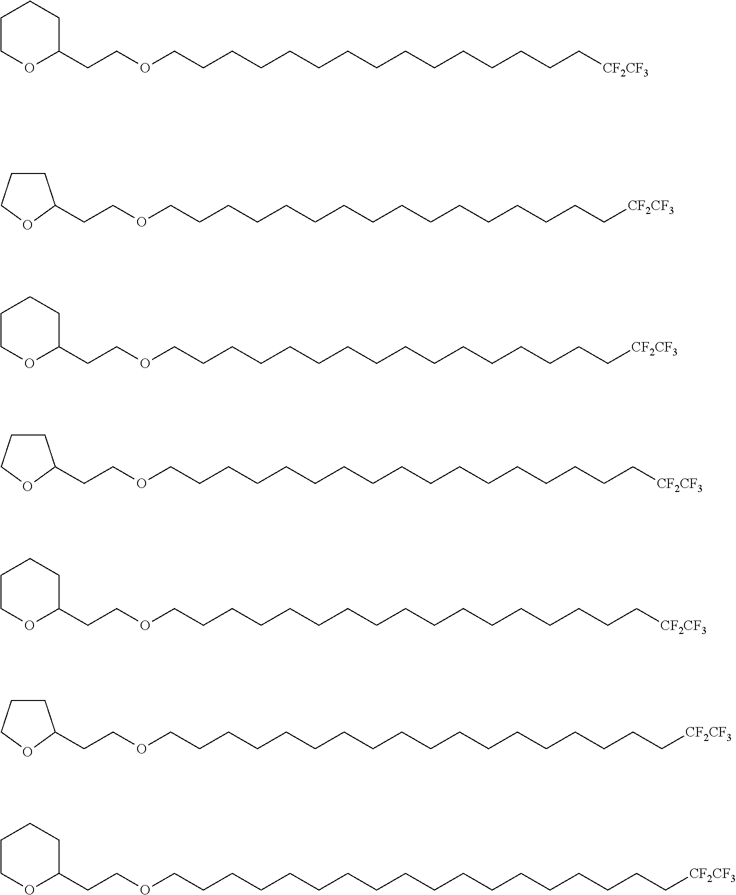

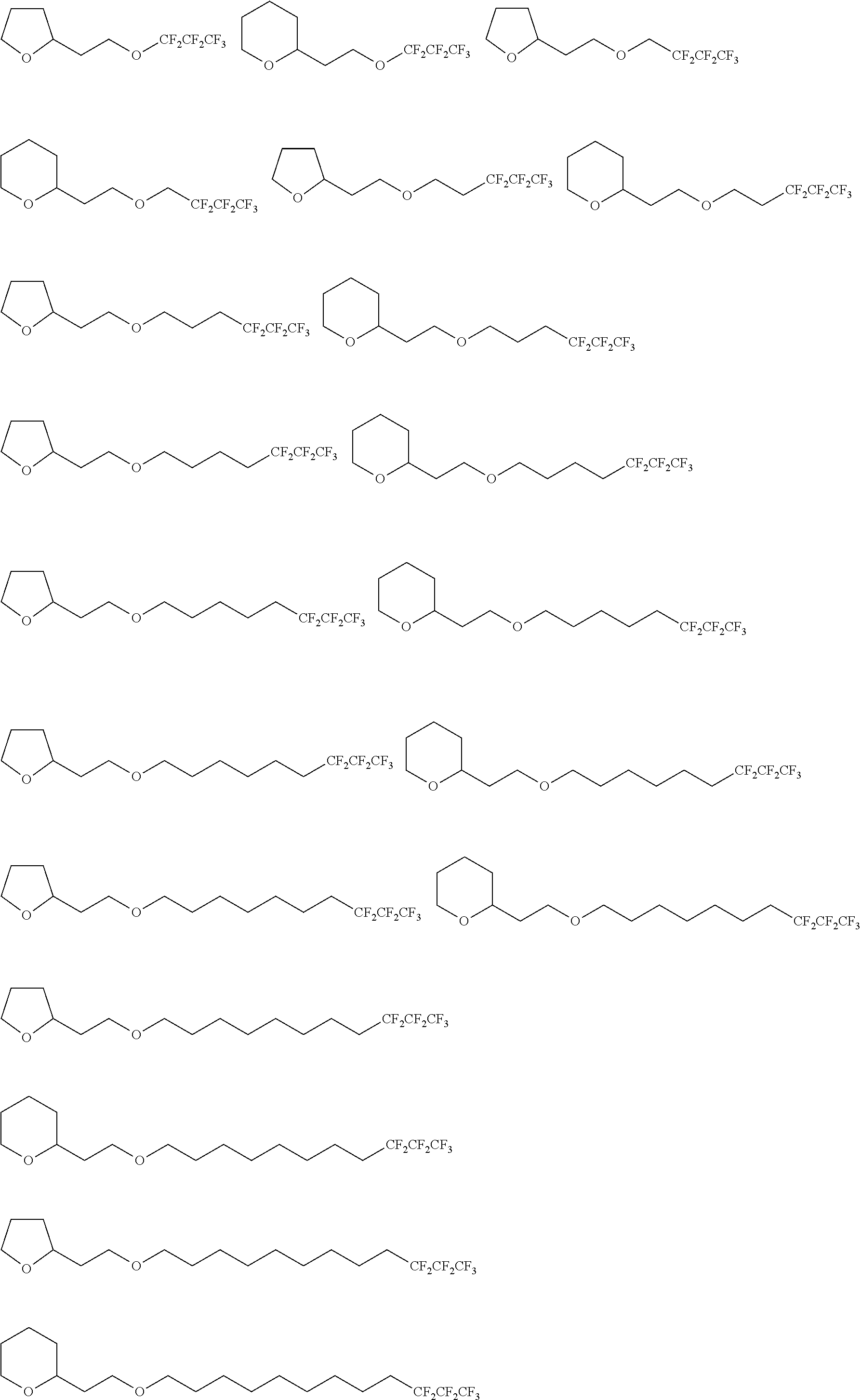

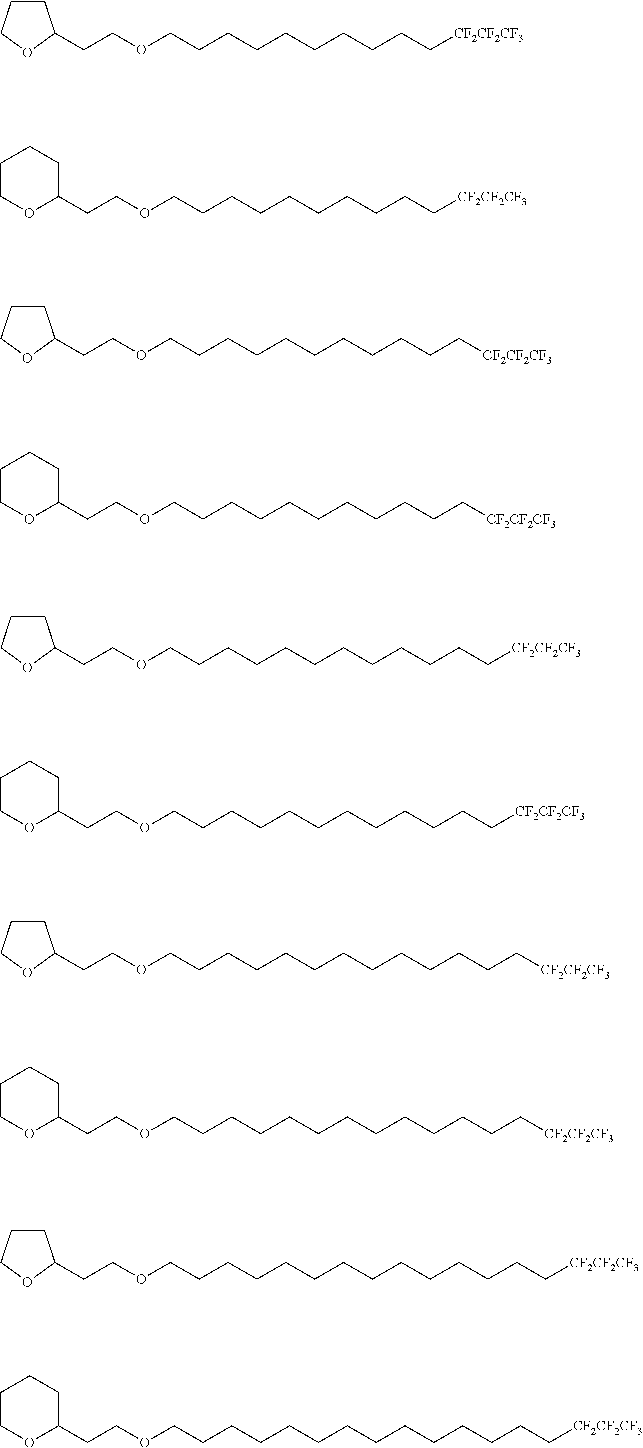

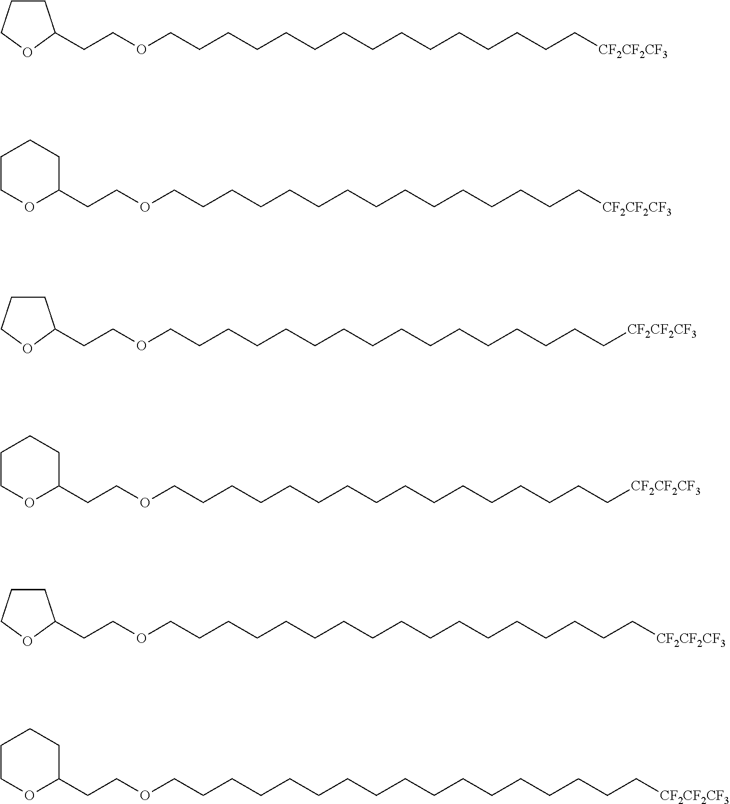

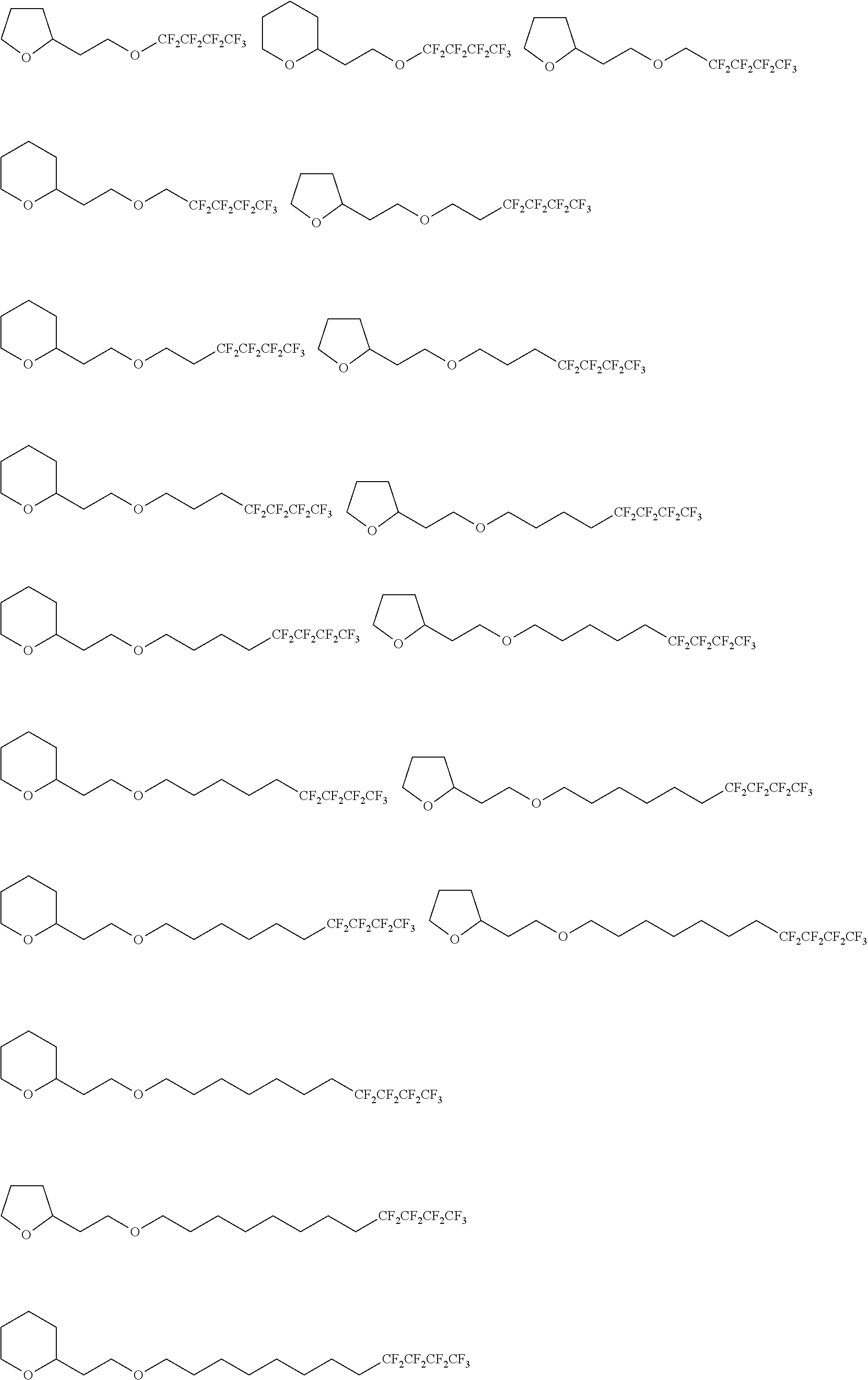

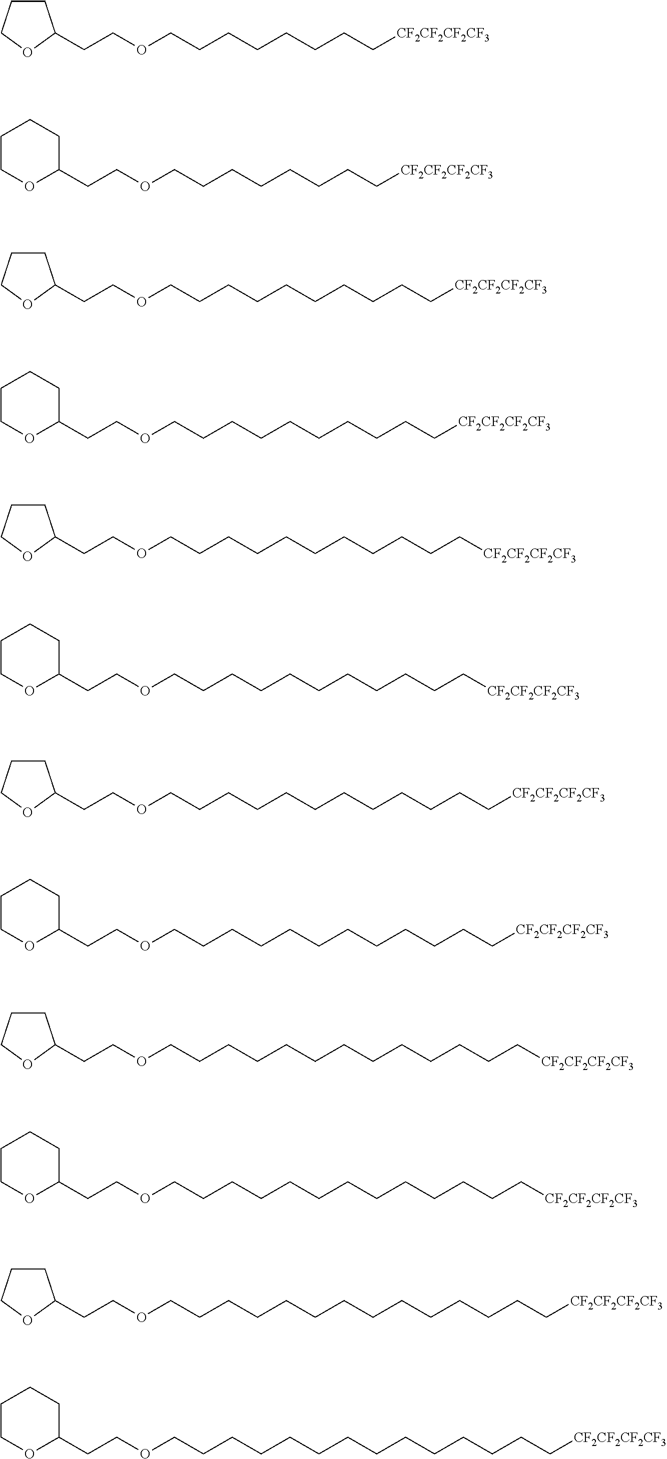

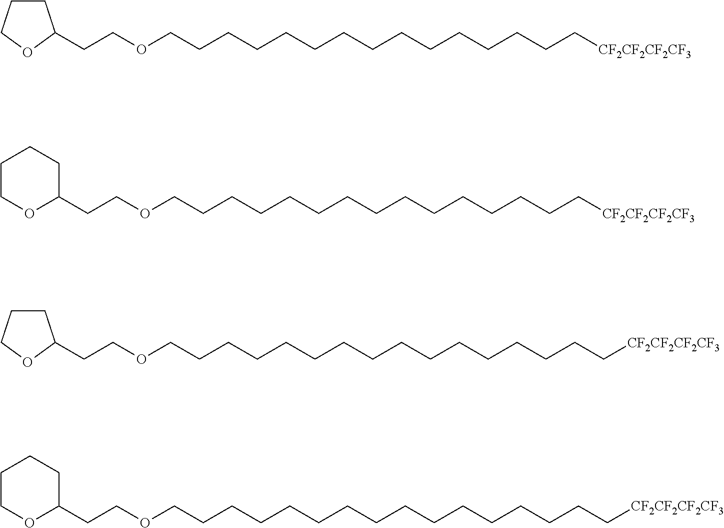

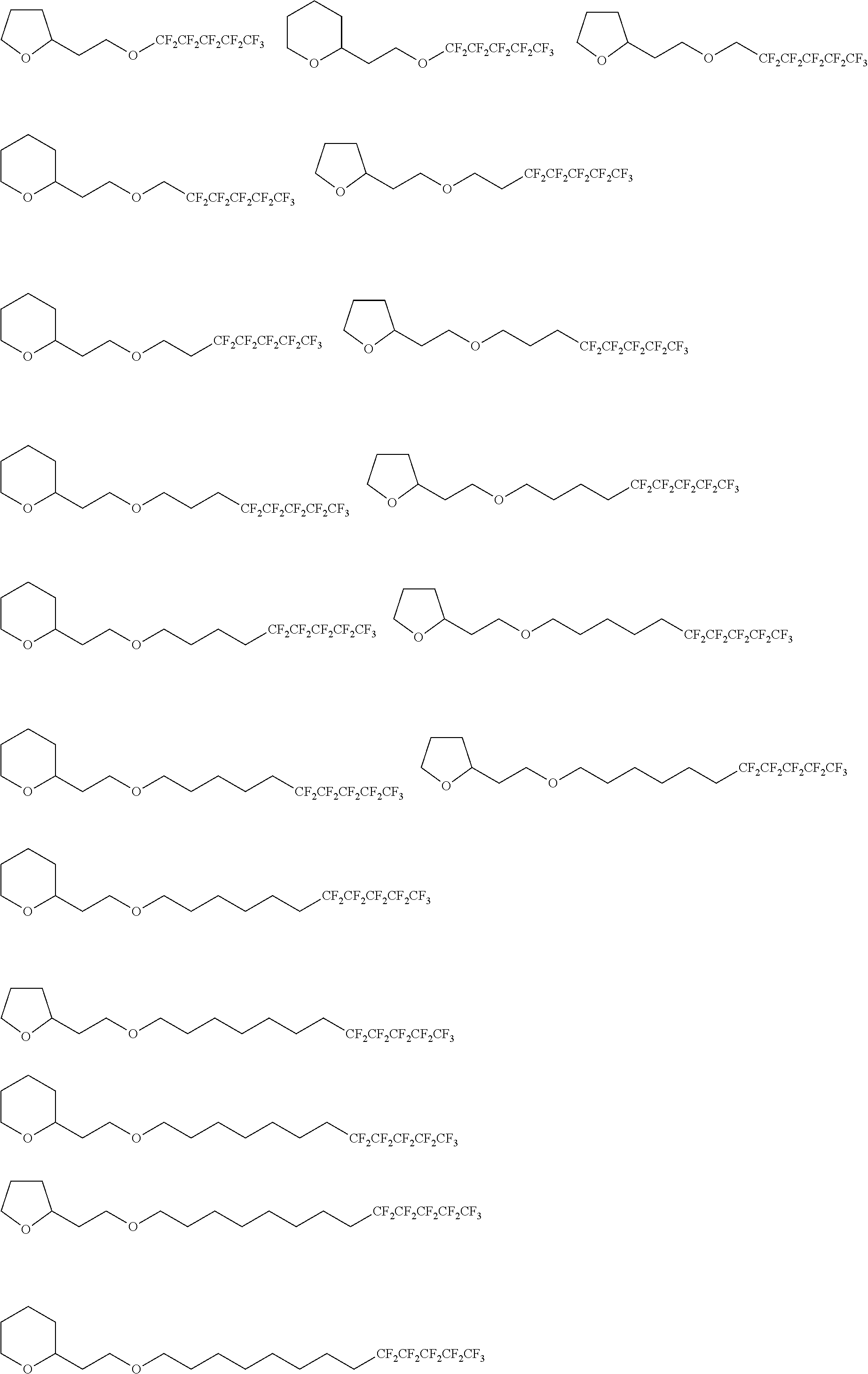

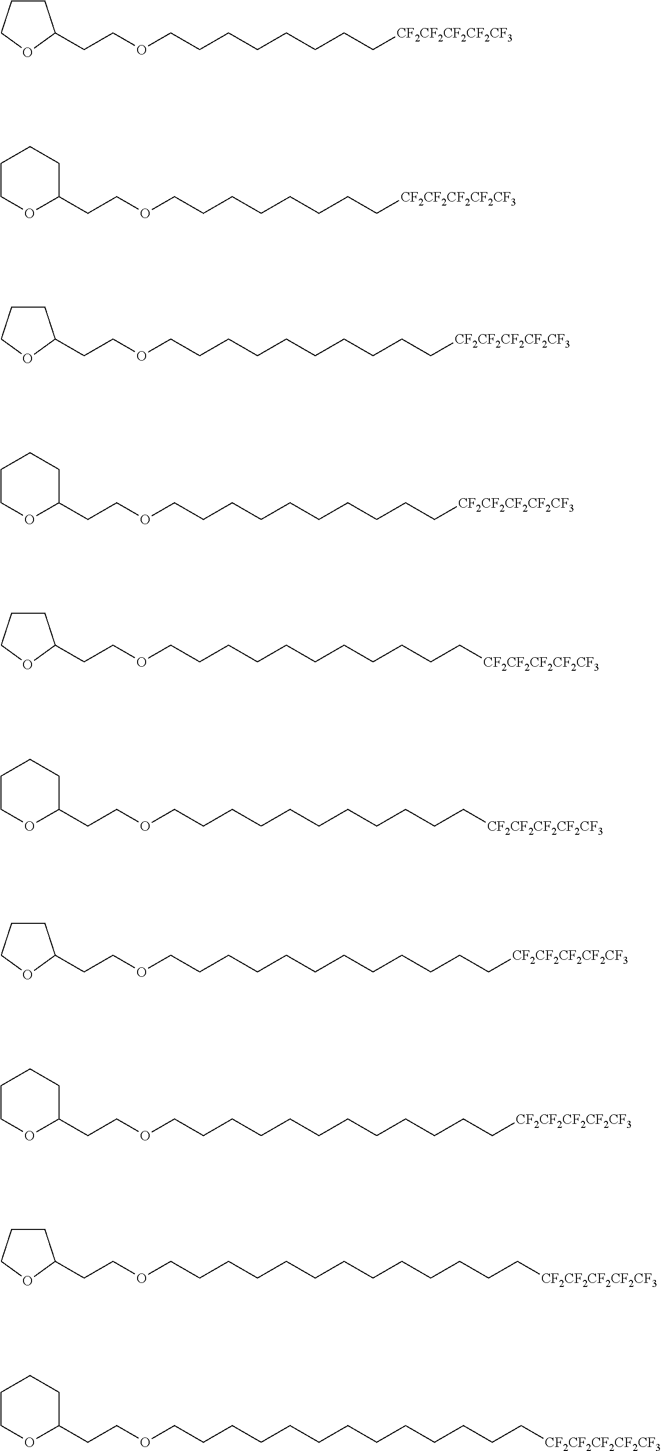

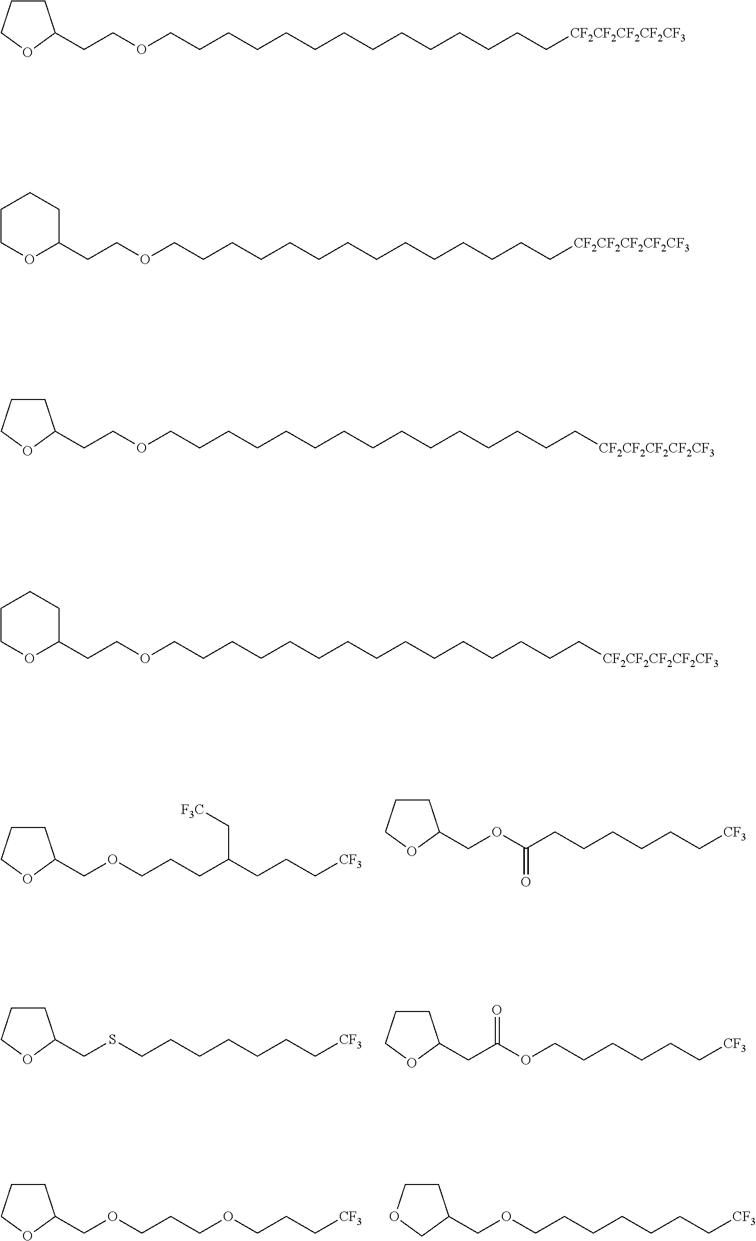

The method for producing the ether compound of the present invention is not limited, and a general method for synthesizing an ether or an acetal is applicable. Examples of such a method include, but not limited to, the following.

I. A method in which an alcohol is reacted with a base such as sodium hydride to activated the alcohol, and then reacted with a halide.

II. A method in which an alcohol is derivatized into an active ester, and then the active eater is reacted with an alcohol in the presence of a base.

III. A method in which an alcohol and an olefin are subjected to an addition reaction in the presence of a base.

IV. A method in which an alcohol and an olefin are subjected to an addition reaction in the presence of an acid.

[2. Electrolyte Solution Composition for Non-Aqueous Battery of the Invention]

The electrolyte solution composition for a non-aqueous battery of the present invention (appropriately referred to hereinbelow as “the electrolyte solution composition of the present invention”) includes an organic solvent, an electrolyte dissolved in the organic solvent, and the ether compound of the present invention.

[2-1. Organic Solvent]

The organic solvent for use may be appropriately selected from solvents that are publicly known as a solvent for a non-aqueous electrolyte solution composition. Examples thereof may include cyclic carbonates without an unsaturated bond, chain carbonates, cyclic ethers without a structure represented by the formula (1), chain ethers, cyclic carboxylic acid esters, chain carboxylic acid esters, and phosphorous-containing organic solvents.

Examples of the cyclic carbonates without an unsaturated bond may include alkylene carbonates having an alkylene group of 2 to 4 carbon atoms, such as ethylene carbonate, propylene carbonate, and butylene carbonate. Among them, ethylene carbonate and propylene carbonate are preferable.

Examples of the chain carbonates may include dialkyl carbonates having an alkyl group of 1 to −4 carbon, atoms, such as dimethyl carbonate, diethyl carbonate, di-n-propyl carbonate, ethyl methyl carbonate, methyl-N-propyl carbonate, and ethyl-n-propyl carbonate. Among them, dimethyl carbonate diethyl carbonate, and ethyl methyl carbonate are preferable.

Examples of the cyclic ethers without a structure represented by the formula (1) may include tetrahydrofuran and 2-methyltetrahydrofuran.

Examples of the chain ethers may include dimethoxy ethane and dimethoxy methane.

Examples of the cyclic carboxylic acid esters may include γ-butyrolactone and γ-valerolactone.

Examples of the chain carboxylic acid esters may include methyl acetate, methyl propionate, ethyl propionate, and methyl butyrate.

Examples of the phosphorous-containing organic solvent may include trimethyl phosphate, triethyl phosphate, dimethyl ethyl phosphate, methyl diethyl phosphate, ethylene methyl phosphate, and ethylene ethyl phosphate.

As the organic solvent, one species may be used alone, or two or more species thereof may be used in combination at any ratio. However it is preferable that two or more species of compounds are used in combination. For example, a solvent having a high dielectric constant such as alkylene carbonates and cyclic carboxylic acid esters, and a solvent having a low viscosity such as dialkyl carbonates and chain carboxylic acid esters are preferably used in combination since thereby high lithium ion conductivity is achieved and high capacity can be obtained.

[2-2. Electrolyte]

As the electrolyte, those which are appropriate in accordance with the type of the non-aqueous battery to which the electrolyte solution composition of the present invention is applied may be used. In the electrolyte solution composition of the present invention, the electrolyte usually exists in a state of being dissolved in an organic solvent as a supporting electrolyte. As the electrolyte, a lithium salt is usually used.

Examples of the lithium salt may include LiPF₆, LiAsF₆, LiBF₄, LiSbF₆, LiAlCl₄, LiClO₄, CF₃SO₃Li, C₄F₉SO₃Li, CF₃COOLi (CF₃CO)₂NLi, (CF₃SO₂)₂NLi, and (C₂F₅SO₂)NLi. Among them, LiPF₆, LiClO₄, CF₃SO₃Li, and —LiBF₄ are preferable, since they have high solubility in an organic solvent and show high degree of dissociation. Use of an electrolyte having a high degree of dissociation increases the lithium ion conductivity. Therefore, the lithium ion conductivity can be adjusted by selecting the type of the electrolyte.

As the electrolyte, one species thereof may be used alone, or two or more species thereof may be used in combination at any ratio.

The concentration of the electrolyte contained in the electrolyte solution composition of the present invention is usually equal to or more than 1% by weight and preferably equal to or more than 0.5% by weight, and usually equal to or less than 3.0% by weight and preferably equal to or less than 20% by weight, with respect to 100% by weight of the electrolyte solution composition of the present invention. Depending on the type of the electrolyte, the electrolyte is usually used in a concentration of 0.5 mol/L to 2.5 mol/L in some cases. If the concentration of the electrolyte is too low or high, the ion conductivity tends to decrease. Usually, the lower the concentration of an electrolyte is, the higher the swelling degree of polymer particles, i.e. the binder (described later), becomes. Therefore, the lithium ion conductivity can be adjusted by adjusting the concentration of the electrolyte.

[2-3. Ether Compound]

The electrolyte solution composition of the present invention includes the ether compound of the present invention. The concentration of the ether compound of the present invention contained in the electrolyte solution composition of the present invention is preferably equal to or more than 0.01% by weight, more preferably equal to or more than 0.05% by weight, and particularly preferably equal to or more than 0.1% by weight, and preferably equal to or less than 30% by weight, more preferably equal to or less than 10% by weight, and particularly preferably equal to or less than 5% by weight, with respect to 100% by weight of the electrolyte solution composition of the present invention. When the concentration of the ether compound of the present invention is set to be equal to or more than the lower limit of the aforementioned range, the charge-discharge cycle at high temperatures can be stabilized more reliably. When the ether compound of the present invention is contained in an amount in a degree within the aforementioned range, sufficient effects can be stably obtained. Accordingly, the upper limit of the aforementioned range is thus determined.

Since the electrolyte solution composition of the present invention includes the ether compound of the present invention, the discharge capacity of the non-aqueous battery having the electrolyte solution composition of the present invention can be enhanced. Furthermore, the stability of the charge-discharge cycle in a high temperature environment of the non-aqueous battery can be improved. Accordingly, it is possible to realize a non-aqueous battery which has high discharge capacity and excellent stability of the charge-discharge cycle at high temperatures.

As described above, the present inventors have found out that the high discharge capacity and the stable charge-discharge cycle at high temperatures of a non-aqueous battery can be achieved at high levels in a balanced manner by using the electrolyte solution composition of the present invention. This is based on the following examinations.

As an additive for suppressing decomposition of an electrolyte solution composition, vinylene carbonate has conventionally been known. It has been considered that vinylene carbonate is decomposed at a reduction potential during charging and discharging, and selectively forms a stable protective film on a surface of a negative electrode active material, thereby suppressing the decomposition of an electrolyte solution. This stable protective film also had a small resistance against insertion and extraction of lithium ions, and showed excellent stability of a charge-discharge cycle of a negative electrode. On the other hand, a compound which generates at a positive electrode a stable protective film having a small resistance against insertion and extraction of lithium ions has not been conventionally known.

In such a circumstance, the present inventors have examined for a compound which is capable of generating a stable protective film having a small resistance against insertion and extraction of lithium ions selectively on a positive electrode. As a result, the ether compound of the present invention has been found out. Then, the present inventors have found out that when such a stable protective film is formed at a positive electrode, the aforementioned battery performances can be elevated at high levels in a balanced manner.

The aforementioned selective properties of the ether compound of the present invention are due to the combination of the cyclic ether skeleton and the specific structure. The compound has excellent stability at the reduction potential, and decomposes at a specific oxidation potential, whereby a stable protective film can be generated. It is considered that this stable protective film has a high polarity that is close to a polarity of an electrolyte solution composition, and therefore the resistance against insertion and extraction of lithium ions decreases.

Thus, the present invention is based on a presumption that an electrolyte solution composition containing the ether compound of the present invention which is capable of forming the desired stable protective film selectively at a positive electrode can realize a non-aqueous battery having a high discharge capacity and excellent stability of a charge-discharge cycle at high temperatures.

[2-4. Other Components]

In addition to the organic solvent, the electrolyte and the ether compound of the present invention, the electrolyte solution composition of the present invention may contain an optional component as long as the effects of the present invention are not significantly impaired. As the optional component, one species may be contained alone, or two or more species thereof may be contained in combination at any ratio.

Examples of the optional component may include a cyclic carbonate ester having an unsaturated bond in the molecule, an overcharge preventing agent, a deoxidizing agent, and a dehydrating agent.

The cyclic carbonate ester having an unsaturated bond in the molecule forms a stable protective film on the surface of a negative electrode. For this reason, when the electrolyte solution composition of the present invention contains acyclic carbonate ester having an unsaturated bond in the molecule, the stability of the charge-discharge cycle of a non-aqueous battery can be further improved. Examples of the cyclic carbonate ester having an unsaturated bond in the molecule may include a vinylene carbonate compound, a vinylethylene carbonate compound, and a methylene ethylene carbonate compound.

Examples of the vinylene carbonate compound may include vinylene carbonate, methylvinylene carbonate, ethylvinylene carbonate, 4,5-dimethylvinylene carbonate, 4,5-diethylvinylene carbonate, fluorovinylene carbonate, and trifluoromethylvinylene carbonate.

Examples of the vinylethylene carbonate compound may include vinylethylene carbonate, 4-methyl-4-vinylethylene carbonate, 4-ethyl-4-vinylethylene carbonate, 4-n-propyl-4-vinylethylene carbonate, 5-methyl-4-vinylethylene carbonate, 4,4-divinylethylene carbonate, and 4,5-divinylethylene carbonate.

Examples of the methylene ethylene carbonate compound may include methylene ethylene carbonate, 4,4-dimethyl-5-methylene ethylene carbonate, and 4,4-diethyl-5-methylene ethylene carbonate.

Among them, vinylene carbonate and vinylethylene carbonate are preferable, and vinylene carbonate is particularly preferable. As the cyclic carbonate ester having an unsaturated bond in the molecule, one species may be used alone, or two or more species thereof may be used in combination at any ratio.

When the electrolyte solution composition of the present invention contains a cyclic carbonate ester having an unsaturated bond in the molecule, the concentration of the cyclic carbonate ester having an unsaturated bond in the molecule in 100% by weight of the electrolyte solution composition of the present invention is usually equal to or higher than 0.01% by weight, preferably equal to or higher than 0.1% by weight, more preferably equal to or than 0.3% by weight, and particularly preferably equal to or higher than 0.5% by weight. By containing the cyclic carbonate ester having an unsaturated bond in the molecule at the aforementioned concentration, an effect of improving cycle property of the non-aqueous battery can be stably exerted. In general, when an electrolyte solution composition contains a cyclic carbonate ester having an unsaturated bond in the molecule, there is a possibility that the amount of gas generation during continuous charging increases. However, such increase in the amount of gas generation can be suppressed by co-use of the cyclic carbonate ester having an unsaturated bond in the molecule with the other compound of the present invention, whereby charge-discharge cycle can be stabilized. However, when the content of the cyclic carbonate ester having an unsaturated bond in the molecule is too large, the amount of gas generation during high temperature storage tends to increase. Therefore, the upper limit of the content is usually equal to or lower than 8% by weight, preferably equal to or lower than 4% by weight, and more preferably equal to or lower than 3% by weight.

When the electrolyte solution composition of the present invention contains a cyclic carbonate ester having an unsaturated bond in the molecule, the ratio (by weight) of the cyclic carbonate ester having an unsaturated bond in the molecule with respect to the ether compound of the present invention is usually equal to or higher than 0.5, preferably equal to or higher than 1, and usually equal to or lower than 80, and preferably equal to or lower than 50. When the ratio of the cyclic carbonate ester having an unsaturated bond in the molecule is too high, the amount of a gas generated during high temperature storage tends to increase. When the ratio is too low, there is a possibility that the effect of stabilizing a charge-discharge cycle cannot be sufficiently exerted.

Examples of the overcharge preventing agent may include aromatic compounds such as biphenyl, alkyl biphenyl, terphenyl, partially hydrogenated terphenyl, cyclohexylbenzene, t-butylbenzene, t-amylbenzene, diphenyl ether, and dibenzofuran; partial fluorides of the aforementioned aromatic compounds such as 2-fluoro biphenyl, o-cyclohexylfluorobenzene, and p-cyclohexylfluorobenzene; and fluorine-containing anisole compounds such as 2,4-difluoroanisole, 2,5-difluoroanisole, and 2,6-difluoroanisole. As the overcharge preventing agent, one species thereof may be used alone, or two or more species thereof may be used in combination at any ratio.

When the electrolyte solution composition of the present invention contains an overcharge preventing agent, the concentration of the overcharge preventing agent in 100% by weight of the electrolyte solution composition of the present invention is usually 0.1% by weight to 5% by weight. By containing an overcharge preventing agent, rupture and ignition of the non-aqueous battery during overcharging and the like can be suppressed.

In general, an overcharge preventing agent has higher reactivity at a positive electrode and a negative electrode than the solvent component of the electrolyte solution composition. Therefore, the overcharge preventing agent has a tendency to react at a site of the electrode having high activity during continuous charging and during high temperature storage. The reaction of the overcharge preventing agent significantly elevates the internal resistance of the non-aqueous battery, and generates a gas, which cause remarkable decrease in charge-discharge cycle property and in charge-discharge cycle property at high temperatures. However, if the overcharge preventing agent is contained in the electrolyte solution composition of the present invention, decrease in charge-discharge cycle property can be suppressed.

Examples of the optional component other than the aforementioned components may include auxiliary agents such as carbonate compounds such as fluoroethylene carbonate, trifluoro propylene carbonate, phenyl ethylene carbonate, erythritan carbonate, spiro-bis-dimethylene carbonate, methoxyethyl-methyl carbonate, and catechol carbonate; carboxylic anhydrides such as succinic anhydride, glutaric anhydride, maleic anhydride, citraconic anhydride, glutaconic anhydride, itaconic anhydride, diglycolic anhydride, cyclohexanedicarboxylic anhydride, cyclopentanetetracarboxylic dianhydrid, and phenylsuccinic anhydride, sulfur-containing compounds such as ethylene sulfite, 1,3-propanesultone, 1,4-butanesultone, methyl methanesulphonate, busulfan, sulfolane, sulfolene, dimethylsulfone, tetramethylthiuram monosulfide, N,N-dimethylmethanesulfonamide, and N,N-diethylmethanesulfonamide; nitrogen-containing compounds such as 1-methyl-2-pyrrolidinone, 1-methyl-2-piperidone, 3-methyl-2-oxazolidinone, 1,3-dimethyl-2-imidazolidinone, and N-methylsuccinimide hydrocarbon compounds such as heptane, octane, and cycloheptane; fluorine-containing aromatic compounds such as fluorobenzene, difluorobenzene, hexafluorobenzene, and benzotrifluoride; 1,6-dioxaspiro[4,4]nonane-2,7-dione; and 12-crown-4-ether. As the auxiliary agent, one species thereof may be used alone, or two or more types thereof may be used in combination at any ratio.

When the electrolyte solution composition of the present invention contains auxiliary agents, the concentration of auxiliary agents in 100% by weight of the electrolyte solution composition of the present invention is usually 0.1% by weight to 5% by weight. By containing these auxiliary agents, capacity maintenance property after high temperature storage and cycle property can be improved.

[2-5. Method for Producing Electrolyte Solution Composition of the Invention]

The electrolyte solution composition of the present invention may be produced by, for example, dissolving an electrolyte and the ether compound of the present invention, as well as an optional component if necessary, in an organic solvent. When producing the electrolyte solution composition of the present invention, it is preferable that each material is previously dehydrated in advance of mixing. It is desirable that the dehydration be carried out until the water content usually becomes equal to or lower than 50 ppm, preferably equal to or lower than 30 ppm.

[3. Binder Composition for Non-Aqueous Battery Electrode of the Invention]

The binder composition for a non-aqueous battery electrode of the present invention (appropriately referred to hereinbelow as “the binder composition of the present invention”) contains an acrylic-based polymer and the ether compound of the present invention. Usually, the binder composition of the present invention also contains a solvent.

[3-1. Acrylic-Based Polymer]

The acrylic-based polymer is a component which functions as a binder in a non-aqueous battery. The binder herein refers to a component which retains an electrode active material in an electrode active material layer. An acrylic-based polymer is an excellent binder because of its excellent binding property with an electrode active material as well as the strength and flexibility of the resulting electrode. Since an acrylic-based polymer is usually a saturated-type polymer which does not have an unsaturated bond in the polymer main chain and is excellent in oxidation resistance during charging, it is especially suitable as a binder for a positive electrode.

An acrylic-based polymer refers to a polymer which contains a monomer unit obtained by polymerizing one or both of an acrylic acid ester and a methacrylic acid ester. Examples of the acrylic acid ester and the methacrylic acid ester may include acrylic acid alkyl esters such as methyl acrylate, ethyl acrylate, n-propyl acrylate, isopropyl acrylate, n-butyl acrylate, t-butyl acrylate, pentyl acrylate, hexyl acrylate, heptyl acrylate, octyl acrylate, 2-ethylhexyl acrylate, nonyl acrylate, decyl acrylate, lauryl acrylate, n-tetradecyl acrylate, and stearyl acrylate; and methacrylic acid alkyl esters such as methyl methacrylate, ethyl methacrylate, n-propyl methacrylate, isopropyl methacrylate, n-butyl methacrylate, t-butyl methacrylate, pentyl methacrylate, hexyl methacrylate, heptyl methacrylate, octyl methacrylate, 2-ethylhexyl methacrylate, nonyl methacrylate, decyl methacrylate, lauryl methacrylate, n-tetradecyl methacrylate, and stearyl methacrylate. Among them, ethyl acrylate, ft-butyl acrylate, hexyl acrylate, and 2-ethylhexyl acrylate are preferable, since they are not eluted in an electrolyte solution and show conductivity of lithium ions due to a proper swelling in the electrolyte solution, and in addition since they are not likely to cause bridging flocculation due to an acrylic-based polymer in dispersion of electrode active materials. As the acrylic acid ester or methacrylic acid ester, one species thereof may be used alone, or two or more species thereof may be used in combination at any ratio. Furthermore, an acrylic acid ester and a methacrylic acid ester may be used in combination. The ratio of the monomer unit, in an acrylic-based polymer, obtained by polymerizing one or both of an acrylic acid ester and a methacrylic acid ester is usually equal to or higher than 40% by weight, preferably equal to or higher than 50% by weight, more preferably equal to or higher than 60% by weight, and usually equal to or lower than 100% by weight.

It is preferable that the acrylic-based polymer includes a monomer unit obtained by polymerizing a monomer which is copolymerizable with a (meth)acrylic acid ester, in addition to a monomer unit obtained by polymerizing a (meth)acrylic acid ester. The “(meth)acryl” herein refers to “acryl” and “methacryl”. Examples of the co-polymerizable monomer may include unsaturated carboxylic acids such as acrylic acid, methacrylic acid, itaconic acid, and fumaric acid; carboxylic acid esters having two or more carbon-carbon double bonds per molecule, such as ethylene glycoldimethacrylate, diethylene glycoldimethacrylate, and trimethylolpropane triacrylate; styrene-based monomers such as styrene, chlorostyrene, vinyltoluene, t-butylstyrene, vinylbenzoic acid; methyl vinylbenzoate, vinylnaphthalene, chloromethylstyrene, hydroxymethyl styrene, α-methylstyrene, and divinylbenzene; amide-based monomers such as acrylamide, N-methylolacrylamide, and acrylamide-2-methylpropanesulfonic acid; α,β-unsaturated nitrile compounds such as acrylonitrile and methacrylonitrile; olefins such as ethylene and propylene; diene-based monomers such as butadiene and isoprene; halogen atom-containing monomers such as vinyl chloride and vinylidene chloride; vinyl esters such as vinyl acetate, vinyl propionate, vinyl butyrate, and vinyl benzoate; vinyl ethers such as methyl vinyl ether, ethyl vinyl ether, and butyl vinyl ether; vinyl ketones such as methyl vinyl ketone, ethyl vinyl ketone, butyl vinyl ketone, hexyl vinyl ketone, and isopropenyl vinyl ketone; and heterocyclic ring-containing vinyl compounds such as N-vinyl pyrrolidone, vinyl pyridine, and vinyl imidazole. As the copolymerizable monomer, one species thereof may be used alone, or two or more species thereof may be used in combination at any ratio.

As the acrylic-based polymer, those which have a crosslink structure may be used and those into which a functional group is introduced by modification may also be used. Examples of the method for introducing a crosslink structure into an acrylic-based polymer may include a method of causing crosslinking by heating or energy, ray irradiation. Examples of the method for modifying an acrylic-based polymer to obtain an acrylic-based polymer capable of crosslinking by heating or energy ray irradiation may include a method of introducing a crosslinkable group into an acrylic-based polymer and a method in which a crosslinking agent is co-used.

Examples of the method of introducing a crosslinkable group in an acrylic-based polymer may include a method of introducing a photo-crosslinkable group into an acrylic-based polymer and a method of introducing a thermal crosslinkable group into an acrylic-based polymer. Among them, a method of introducing a thermal crosslinkable group into an acrylic-based polymer is preferable since, by performing a heating treatment on an electrode active material layer after formation of the electrode active material layer, the binder can be crosslinked in the electrode active material layer, and dissolution of the binder into the electrolyte solution can thus be suppressed, so that a tough and flexible electrode active material layer can be obtained. In the case where a thermal crosslinkable group is introduced in an acrylic-based polymer, for example, possible methods therefor may be a method in which a monofunctional monomer having one olefinic double bond and a thermal crosslinkable group is used, and a method in which a multifunctional monomer having two or more olefinic double bonds per molecule is used.

Examples of the thermal crosslinkable group that the monofunctional monomer having one olefinic double bond has may include one selected from the group consisting of an epoxy group, a hydroxyl group, an N-methylolamide group, an oxetanyl group, and an oxazoline group. The epoxy group is more preferable since thereby crosslinking and crosslinking density can be easily adjusted. As the crosslinkable group, one species thereof may be used alone, or two or more species thereof may be used in combination at any ratio.

Examples of the monomer containing an epoxy group may include a monomer containing a carbon-Carbon double bond and an epoxy group, and a monomer containing a halogen atom and an epoxy group.

Examples of the monomer containing a carbon-carbon double bond and an epoxy group may include unsaturated glycidyl ethers such as vinyl glycidyl ether, allyl glycidyl ether, butenyl glycidyl ether, and o-allylphenyl glycidyl ether; monoepoxides of diene or polyene such as butadiene monoepoxide, chloroprene monoepoxide, 4,5-epoxy-2-pentene, 3,4-epoxy-1-vinylcyclohexene, and 1,2-epoxy-5,9-cyclododecadiene; alkenyl epoxides such as 3,4-epoxy-1-butane, 1,2-epoxy-5-hexene, and 1,2-epoxy-9-decene; and glycidyl esters of unsaturated carboxylic acid such as glycidyl acrylate, glycidyl methacrylate, glycidyl crotonate, glycidyl-4-heptenoate, glycidyl solvate, glycidyl linolenate, glycidyl-4-methyl-3-pentenoate, glycidyl ester of 3-cyclohexenecarboxylic acid, and glycidyl ester of 4-methyl-3-cyclohexenecarboxylic acid.

Examples of the monomer having a halogen atom and an epoxy group may include epihalohydrin such as epichlorohydrin, epibromohydrin, epiiodohydrin, epifluorohydrin, and β-methylepichlorhydrin; p-chlorostyrene oxide; and dibromophenyl glycidyl ether.

Examples of the monomer containing an N-methylolamide group may include (meth)acrylamides having a methylol group such as N-methylol(meth)acrylamide.

Examples of the monomer containing an oxetanyl group may include 3-((meth)acryloyloxymethyl)oxetane, 3-((meth)acryloyloxymethyl)-2-trifluoromethyloxetane, ((meth)acryloyloxymethyl)-2-phenyloxetane, 2-((meth)acryloyloxymethyl)oxetane, and ((methacryloyloxymethyl)-4-trifluoromethyloxetane.

Examples of the monomer containing an oxazoline group may include 2-vinyl-2-oxazoline, 2-vinyl-4-methyl-2-oxazoline, 2-vinyl-5-methyl-2-oxazoline, 2-isopropenyl-2-oxazoline, 2-isopropenyl-4-methyl-2-oxazoline, 2-isopropenyl-5-Methyl-2-oxazoline, and 2-isopropenyl-5-ethyl-2-oxazoline.

Examples of the multifunctional monomer having two or more olefinic double bonds per molecule may include allyl acrylate, allyl methacrylate, trimethylolpropane-triacrylate, trimethylolpropane-methacrylate, dipropylene glycol diallyl ether, polyglycol diallyl ether, triethyleneglycol divinyl ether, hydroquinone diallyl ether, tetraallyloxyethane, other allyl or vinyl ether of polyfunctional alcohol, tetraethylene glycol diacrylate, triallylamine, trimethylolpropane-diallyl ether, methylenebisacrylamide, and divinyl benzene. Allyl acrylate, allyl methacrylate, trimethylolpropane-triacrylate, and trimethylolpropane-methacrylate are particularly preferable.

Among them, a monomer containing an epoxy group and a multifunctional monomer having two or more olefinic double bonds per molecule are preferable since therewith crosslinking density can be easily improved. A multifunctional monomer having two or more olefinic double bonds per molecule is further preferable in terms of crosslinking density improvement and its high copolymerization property. Especially, acrylate and methacrylate having an allyl group such as allyl acrylate and allyl methacrylate are preferable.

As the acrylic-based polymer, one species may be used alone, or two or more species thereof may be used in combination at any ratio.

Although the glass transition temperature (Tg) of the acrylic-based polymer may be appropriately selected depending on the intended use, the temperature is usually equal to or higher than −150° C., preferably equal to or higher than −50° C. and further preferably equal to or higher than −35° C., and usually equal to or lower than +100° C., preferably equal to or lower than +25° C., and further preferably equal to or lower than +5° C. When the glass transition temperature Tg of an acrylic-based polymer falls within this range, properties such as flexibility, binding properties, and winding properties of an electrode, as well as adhesion between an electrode active material layer and a current collector are well balanced, and thus being favorable.

The method for producing the acrylic-based polymer to be employed in the present invention is not particularly limited, and example thereof may include any method such as a solution polymerization method, dispersion polymerization, a suspension polymerization method, a bulk polymerization method, and an emulsion polymerization method. The polymerization reaction to be employed may be any reaction such as ion polymerization, radical polymerization, and living radical polymerization. Examples of the polymerization initiator for use in the polymerization may include organic peroxides such as lauroyl peroxide, diisopropyl peroxydicarbonate, di-2-ethylhexyl peroxydicarbonate, t-butyl peroxypivalate, and 3,3,5-trimethyl hexanoyl peroxide, azo compounds such as α,α′-azobisisobutyronitrile, ammonium persulfate, and potassium persulfate. Among them, dispersion polymerization, emulsion polymerization, and suspension polymerization in an aqueous solvent are preferable since it is preferable that the acrylic-based polymer is in a particle dispersion state.

In the binder composition of the present invention, an acrylic-based polymer may sometimes exist as particles. Usually, when producing an electrode, a binder such as an acrylic-based polymer is often prepared in a state of a solution or a dispersion liquid in which the binder is dissolved or dispersed in a solvent. The binder composition of present invention is such a solution or dispersion liquid. When the binder composition of the present invention is a dispersion liquid, an acrylic-based polymer is usually dispersed as particles in the composition. In this case, the mean particle size of the acrylic-based polymer particles is preferably equal to or larger than 50 nm and more preferably equal to or larger than 70 nm, and preferably equal to or smaller than 500 nm and more preferably equal to or smaller than 400 nm. When the mean particle size falls within this range, strength and flexibility of the obtained electrode become favorable. As the mean particle size of particles of an acrylic-based polymer, a 50% volume cumulative diameter may be employed. The 50% volume cumulative diameter may be calculated by measuring a particle size distribution using a laser diffraction technique.

Especially, it is preferable that the acrylic-based polymer exists as particles, and the binder composition is in a state of a dispersion liquid since thereby the acrylic-based polymer does not coat the active material surface, and therefore does not inhibit formation of the stable protective film.

The amount of the acrylic-based polymer in the binder composition of the present invention is usually equal to or more than 5% by weight, preferably equal to or more than 15% by weight, and more preferably equal to or more than 30% by weight, and usually equal to or less than 70% by weight, preferably equal to or less than 65% by weight, more preferably equal to or less than 60% by weight, with respect to 100% by weight of the binder composition of the present invention. This can provide favorable workability during production of a slurry composition for a non-aqueous battery electrode of the present invention (appropriately referred to hereinbelow as “the slurry composition of the present invention”).

[3-2. Ether Compound]

The binder composition of the present invention contains the ether compound of the present invention. The concentration of the ether compound of the present invention contained in the binder composition of the present invention is preferably equal to or larger than 1 part by weight, more preferably equal to or larger than 3 Parts by weight, and particularly preferably equal to or larger than 5% by weight, and preferably equal to or less than 100 parts by weight, more preferably equal to or less than 80 parts by weight, particularly preferably equal to or less than 50 parts by weight, with respect to 100 parts by weight of the acrylic-based polymer of the present invention. By setting the concentration of the ether compound of the present invention to be not less than the lower limit of the aforementioned range, the charge-discharge cycle at high temperatures can be stabilized more reliably. When the ether compound of the present invention is contained in an amount in a degree within the aforementioned range, sufficient effects can be stably obtained. Accordingly, the upper limit of the aforementioned range is thus determined.

Since the binder composition of the present invention contains the ether compound of the present invention, the discharge capacity of the non-aqueous battery to which the binder composition of the present invention is applied can be increased. Furthermore, the stability of the charge-discharge cycle of the non-aqueous battery in a high temperature environment can be improved. That makes it possible to realize a non-aqueous battery having a large discharge capacity and excellent stability of the charge-discharge cycle at high temperatures.

Since the acrylic-based polymer is excellent in oxidation resistance during charging and therefore does not inhibit formation of the stable protective film as described above, the combination of the ether compound of the present invention especially with the acrylic-based polymer among the binders can effectively suppress the reduction in discharge capacity by the ether compound.

[3-3. Solvent]

The binder composition of the present invention usually contains a solvent. Usually, an appropriate solvent is selected depending on the type of the binder contained in the binder composition. The solvent is roughly classified into an aqueous solvent and a non-aqueous solvent. Water is usually used as the aqueous solvent. On the other hand, as the non-aqueous solvent, an organic solvent is usually used. Especially, N-methyl pyrrolidone (NMP) is preferable. As the solvent, one species thereof may be used alone, or two or more species thereof may be used in combination at any ratio. Especially, since the binder composition is preferably a particle dispersion liquid of at acrylic-based polymer, the solvent is preferably an aqueous solvent, and particularly preferably water.

[3-4. Other Component]

In addition to the acrylic-based polymer, the ether compound of the present invention, and the solvent, the binder composition of the present invention may contain an, optional component as long as the effects of the present invention are not significantly impaired. The binder composition of the present invention may include only one species of the optional component, or two or more species thereof.

For example, the binder composition of the present invention may include a binder other than the acrylic-based polymer. As the binder other than the acrylic-based polymer, a variety of binders contained in an electrode, which will be described later, may be used. Especially, a fluorine-based polymer or a diene-based polymer is preferable. The amount of the binder other than the acrylic-based polymer may be determined in a range such that the solid content concentration of the binder composition of the present invention is usually equal to or higher than 15% by weight, preferably equal to or higher than 20% by weight, and more preferably equal to or higher than 30% by weight, and usually equal to or lower than 70% by weight, preferably equal to or lower than 65% by weight, and more preferably equal to or lower than 60% by weight. When the solid content concentration falls within the aforementioned range, workability during the production of the slurry composition of the present invention is favorable. However, the amount of the binder other than the acrylic-based polymer in the binder composition of the present invention is preferably equal to or less than 30 parts by weight, more preferably equal to or less than 20 parts by weight, particularly preferably equal to or less than 10 parts by weight, with respect to 100 parts by weight of the acrylic-based polymer.

[3-5. Method for Producing Binder Composition for Non-Aqueous Battery Electrode of the Invention]

The method for producing the binder composition of the present invention is not limited. When at aqueous solvent is used as the solvent, the binder composition may be produced by, for example, emulsion-polymerization in water of the monomer of the acrylic-based polymer and, if necessary, the monomer of the co-using binder. When a non-aqueous solvent is used as the solvent, the binder composition may be produced by, for example, substituting the solvent of the aforementioned binder composition using an aqueous solvent with an organic solvent. The binder composition of the present invention contains the ether compound of the present invention, which may be mixed either before or after the aforementioned polymerization.

[4. Slurry Composition for Non-Aqueous Battery Electrode of the Invention]

The slurry composition for a non-aqueous battery electrode of the present invention (i.e. the slurry composition of the present invention) contains an electrode active material and the binder composition of the present invention. Therefore, the slurry composition of the present invention contains at least an electrode active material, an acrylic-based polymer, and the ether compound of the present invention. Usually, the slurry composition of the present invention also contains a solvent.

[4-1. Electrode Active Material]

As the electrode active material, an appropriate one may be used depending on the type of a non-aqueous battery. In the following description, the electrode active material for a positive electrode is appropriately referred to as a “positive electrode active material”, and the electrode active material for a negative electrode is referred to as a “negative electrode active material”. Since a preferred non-aqueous battery in the present invention may be a lithium secondary battery and a nickel metal hydride secondary battery, the following description will discuss electrode active materials suitable for a lithium secondary battery and a nickel metal hydride secondary battery.

Firstly, the types of the electrode active material for a lithium secondary battery will be described.

A positive electrode active material for a lithium secondary battery is roughly classified into those composed of an inorganic compound and those composed of an organic compound. Examples of the positive electrode active material composed of an inorganic compound may include transition metal oxides, composite oxides of lithium and transition metal, and transition metal sulfides. Examples of the transition metal may include Fe, Co, Ni, and Mn. Specific examples of the positive electrode active material composed of an inorganic material may include lithium-containing composite metal oxides such as LiCoO₂, LiNiO₂, LiMnO₂, LiMn₂O₄, LiFePO₄, and LiFeVO₄; transition metal sulfides such as TiS₂, TiS₃, and amorphous MoS₂; and transition metal oxides such as Cu₂V₂O₃, amorphous V₂O—P₂O₅, MoO₃, V₂O₅, and V₆O₁₃. On the other hand, specific examples of the positive electrode active material composed of an organic compound may include electroconductive polymer compounds such as polyacetylene and poly-p-phenylene. Furthermore, a positive electrode active material composed of a composite material that is a combination of an inorganic compound and an organic compound may also be used. For example, an iron-based oxide May be subjected to reduction firing in the presence of a carbon source material to prepare a composite material coated with carbon materials. The obtained composite material may be used as a positive electrode active material. An iron-based oxide tends to have poor electrical conductivity. However, by it may be used as a high performance positive electrode active material

By forming such a composite material. Further, those obtained by partial element substitution of the aforementioned compound may also be used as a positive electrode active material.

As the positive electrode active material, one species thereof may be used alone, or two or more species thereof may be used in combination at any ratio. In addition, a mixture of the aforementioned inorganic compound and organic compound may also be used as a positive electrode active material.

Examples of the negative electrode active material for a lithium secondary battery may include carbonaceous materials such as amorphous carbon, graphite, natural graphite, mesocarbon microbeads, and pitch-based carbon fiber; and electroconductive polymer compounds such as polyacene. The examples may also include metals such as silicon, tin, zinc, manganese, iron, and nickel as well as alloys thereof; oxides of these metals and alloys; and sulfates of these metals and alloys. The examples may also include metal lithium; lithium alloys such as Li—Al, Li—Bi—Cd, and Li—Sn—Cd; and lithium transition metal nitrides. As an electrode active material, a material having a surface to which an electroconductivity imparting material adheres by a mechanical modifying method may also be used. As the negative electrode active material, one species thereof may be used alone, or two or more species thereof may be used in combination at any ratio.

Subsequently, the types of the electrode active material for a nickel metal hydride secondary battery will be described.

Examples of the positive electrode active material for a nickel metal hydride secondary battery may include nickel hydroxide particles. The nickel hydroxide particles may contain cobalt, zinc, cadmium, or the like in a solid solution state. The nickel hydroxide particles may have a surface coated with an alkaline-heat-treated cobalt compound. Further, the nickel hydroxide particles may contain additives including yttrium oxide, cobalt compounds such as cobalt oxide, metal cobalt, and cobalt hydroxide; zinc compounds such as metal zinc, zinc oxide, and zinc hydroxide; and rare earth compounds such as erbium oxide. As the positive electrode active material, one species thereof may be used alone, or two or more species thereof may be used in combination at any ratio.

As the negative electrode active material for a nickel metal hydride secondary battery, hydrogen-absorption alloy particles are usually used. The hydrogen-absorption alloy particles are not particularly limited as long as they can absorb hydrogen which is electrochemically generated in the electrolyte solution composition of the present invention during charging of a non-aqueous battery, and also can easily release the absorbed hydrogen during discharging. Particles selected from the group consisting of AB5 type-based, TiNi-based, and TiFe-based hydrogen-absorption alloys are particularly preferable. Specific examples thereof may include LaNi₅, MmNi₅ (MM is a misch metal), and LmNi₅ (Lm is one or more species selected from rare earth elements including La), as well as multielement-hydrogen-absorption alloy particles in which a part of Ni in these alloys is substituted with one or more species of element selected from the group consisting of Al, Mn, Co, Ti, Cu, Zn, Zr, Cr, and B. In particular, hydrogen-absorption alloy particles having a composition represented by a general formula: LmNi_{w}CoₓMn_{y}Al_{z} (atom ratio values w, x, y, and z are positive numbers satisfying 4.80≦w+x+y+z≦5.40) is suitable since micronization associated with progress of charge-discharge cycle is suppressed and thereby the charge-discharge cycle life is improved. As the negative electrode active material, one species thereof may be used alone, or two or more species thereof may be used in combination at any ratio.

In both a lithium secondary battery and a nickel metal hydride secondary battery, the particle size of an electrode active material may be appropriately selected depending on configuration requirements of the non aqueous battery.

From the viewpoints of improvement of battery properties as rate property and cycle property, the 50% volume cumulative diameter of the positive electrode active material is usually equal to or larger than 0.1 μm and preferably equal to or larger than 1 μm, and usually equal to or smaller than 50 μm and preferably equal to or smaller than 20 μm.

From the viewpoints of improvement of battery properties such as initial efficiency, rate properties, and cycle property, the 50% volume cumulative diameter of the negative electrode active material is usually equal to or larger than 1 μm and preferably equal to or larger than 15 μm, and usually equal to or smaller than 50 μm and preferably equal to or smaller than 30 μm.

When the 50% volume cumulative diameters of the positive electrode active material and the negative electrode active material fall within the aforementioned ranges, a secondary battery having excellent rate property and cycle property can be realized, and the slurry composition and electrode of the present invention can be easily handled during production.

[4-2 Acrylic-Based Polymer]

The acrylic-based polymer contained in the slurry composition of the present invention is the same as that described in the section of the binder composition of the present invention. However, in the slurry composition of the present invention, the amount of the acrylic-based polymer with respect to 100 parts by weight of the electrode active material is preferably equal to or more than 0.1 parts by weight, more preferably equal to or more than 0.2 parts by weight, and particularly preferably equal to or more than 0.5 parts by weight, and preferably equal to or less than 10 parts by weight, more preferably equal to or less than 5 parts by weight, and particularly preferably equal to or less than 3 parts by weight. When the amount of the acrylic-based polymer falls within the aforementioned range, it is possible to stably prevent the electrode active material from removal off the electrode without inhibiting battery reaction.

[4-3. Ether Compound]

The slurry composition of the present invention contains the ether compound of the present invention. However, in the slurry composition of the present invention, the amount of the ether compound of the present invention with respect to 100 parts by weight of the electrode active material is preferably equal to or more than 0.01 parts by weight, more preferably equal to or more than 0.1 parts by weight, and particularly preferably equal to or more than 0.2 parts by weight, and preferably equal to or less than 5 parts by weight, more preferably equal to or less than 3 parts by weight, and particularly preferably equal to or less than 2 parts by weight. When the concentration of the ether compound of the present invention is made equal to or more than the lower limit of the aforementioned range, the charge-discharge cycle at high temperatures can be stabilized more reliably. When the ether compound of the present invention in an amount falling within the aforementioned range is contained, sufficient effects can be stably obtained. Accordingly, the upper limit of the aforementioned range is thus determined.

The slurry composition of the present invention containing the ether compound of the present invention can increase the discharge capacity of the non-aqueous battery to which the slurry composition of the present invention is applied. Furthermore, the stability of the charge-discharge cycle of the non-aqueous battery in a high temperature environment can be improved. Accordingly, it is possible to realize a non-aqueous battery having high discharge capacity and excellent stability of the charge-discharge cycle at high temperatures.

The present inventors have found out that use of the slurry composition of the present invention can realize a non-aqueous battery having high discharge capacity and stable charge-discharge cycle at high temperatures at high levels in a balanced manner. This is based on a consideration that, in view of the aforementioned properties attributable to the combination of the cyclic ether skeleton and the specific structure which the ether compound of the present invention has, it can be technically understood that even when the ether compound is used in a slurry of a binder for an electrode, the effects of the present invention, in which a non-aqueous battery having high discharge capacity and excellent stability of charge-discharge cycle at high temperatures can be realized, can also be sufficiently obtained in the same manner as the case wherein the ether compound is used in an electrolyte solution. The inventors have also confirmed that such effects can be sufficiently obtained.

[4-4. Solvent]

Usually, the slurry composition of the present invention contains a solvent. As the solvent of the slurry composition of the present invention, it is possible to select a solvent in which a binder such as the acrylic-based polymer is dissolved or dispersed into a particulate form. When the solvent in which a binder is dissolved is used, the binder is absorbed on the surface, and thereby dispersion of an electrode active material and other components is stabilized. It is preferable to select a specific type of solvent in view of drying speed and environmental factors.

As the solvent of the slurry composition of the present invention, either water and an organic solvent may be used. Examples of the organic solvent may include cyclic aliphatic hydrocarbons such as cyclopentane and cyclohexane; aromatic hydrocarbons such as toluene and xylene; ketones such as ethyl methyl ketone and cyclohexanone; esters such as ethyl acetate, butyl acetate, γ-butyrolactone, and ∈-caprolactone; acylonitriles such as acetonitrile and propionitrile; ethers such as tetrahydrofuran and ethylene glycol diethyl ether; alcohols such as methanol, ethanol, isopropanol, ethylene glycol, and ethylene glycol monomethyl ether; and amides such as N-methylpyrrolidone and N,N-dimethylformamide. Among them, since water is preferable as the aforementioned solvent for a binder composition, it is particularly preferable that the solvent of the slurry composition is also water. Further, the solvent of the binder composition of the present invention as it is may be used as the solvent of the slurry composition of the present invention. As the solvent of the slurry composition of the present invention, one species thereof may be used alone, or two or more species thereof may be used in combination at any ratio.

The amount of the solvent in the slurry composition of the present invention may be adjusted depending on the type of the electrode active material, the binder, and the like, so as to have a viscosity suitable for coating process. Specifically, the amount of the solvent is adjusted so that the solid content concentration that is the total amount of the electrode active material and the binder (including the acrylic-based polymer), as well as optional components that may be contained if necessary in the slurry composition of the present invention is preferably equal to or more than 30% by weight and more preferably equal to or more than 40% by weight, and preferably equal to or less than 90% by weight and more preferably equal to or less than 80% by weight.

[4-5. Other Component]

In addition to the electrode active material, the acrylic-based polymer the ether compound of the present invention and the solvent, the slurry composition of the present invention may contain an optional component as long as the effects of the invention are not significantly impaired. The slurry composition of the present invention may contain only one species of the optional component, or may contain two or more species thereof.

For example, the slurry composition of the present invention may contain a thickening agent. As the thickening agent, a polymer soluble in the solvent of the slurry composition of the present invention is usually used. Examples of the thickening agent may include cellulose-based polymers such as carboxymethyl cellulose, methylcellulose, and hydroxypropylcellulose, and ammonium salts and alkali metal salts thereof; (modified) poly(meth)acrylic acid, and ammonium salts and alkali metal salts thereof; polyvinyl alcohols such as (modified) polyvinyl alcohol, copolymers of acrylic acid or acrylic acid salt and vinyl alcohol, and copolymers of maleic anhydride, or maleic acid or a fumaric acid, and vinyl alcohol; and polyethylene glycol, polyethylene oxide, polyvinylpyrrolidone, modified polyacrylic acid, oxidized starch, starch phosphate, casein, and a variety of modified starches. In the present invention, “(modified)poly” means “unmodified poly” or “modified poly”. As the thickening agent, one species thereof may be used alone, or two or more species thereof may be used in combination at any ratio.

The using amount of the thickening agent is preferably 0.5 parts by weight to 1.5 parts by weight with respect to 100 parts by weight of the electrode active material. When the using amount of the thickening agent falls within this range, coating property of the slurry composition of the present invention become favorable, and it is thus possible to achieve good adhesion of the electrode active material layer and the current collector.

For example, the slurry composition of the present invention may contain an electroconductivity imparting material (also referred to as a electroconductive agent). Examples of the electroconductivity imparting material may include electroconductive carbon such as acetylene black, Ketjen black, carbon black, graphite, vapor grown carbon fiber, and carbon nanotube; carbon powders such as graphite; and fibers and foil of a variety of metals. By using the electroconductivity imparting material, mutual electrical contact among the electrode active materials can be improved. In particular, when used in a lithium secondary battery, discharge rate property can be improved.

For example, the slurry composition of the present invention may contain a reinforcing material. Examples of the reinforcing material may include a variety of inorganic and organic fillers having a spherical shape, a plate shaped, a rod shaped, or a fibrous shape.

Each of the using amount of the electroconductivity imparting material and the using amount of the reinforcing agent with respect to 100 parts by weight of an electrode active material is usually equal to or more than 0 parts by weight and preferably equal to or more than 1 part by weight, and usually equal to or less than 20 parts by weight and preferably equal to or less than 10 parts by weight.

Furthermore, in order to improve the stability and lifetime of the non-aqueous battery of the present invention, the slurry composition of the present invention may contain, other than the aforementioned components, trifluoropropylene carbonate, vinylene carbonate, catechol carbonate, 1,6-dioxaspiro[4,4]nonane-2,7-dione, 12-crown-4-ether and the like.

The slurry composition of the present invention may also contain an optional component that the binder composition of the present invention may contain.

[4-6. Method for Producing Slurry Composition for Non-Aqueous Battery Electrode of the Invention]

The slurry composition of the present invention may be obtained by, for example, mixing the electrode active material, the acrylic-based polymer, the ether compound of the present invention, the solvent, and an optional component that is used if necessary. However, since the slurry composition of the present invention is usually produced by using the binder composition of the present invention, when the solvent of the binder composition of the present invention can be used as the solvent of the slurry composition of the present invention, the solvent of the slurry composition of the present invention does not have to be mixed in addition to the solvent of the binder composition of the present invention.

The mixing order of each component is not particularly limited. For example, the aforementioned respective components may be supplied to a mixer together at a time and then mixed simultaneously. However, when the electrode active material, the acrylic-based polymer, the ether compound of the present invention, the solvent, the electroconductivity imparting material, and the thickening agent are mixed as components of the slurry composition of the present invention, it is preferable that the electroconductivity imparting material and the thickening agent are mixed in the solvent to disperse the electroconductivity imparting material in a form of fine particles, and then the ether compound of the present invention, the acrylic-based polymer, and the electrode active material are mixed in this mixture. By this procedure, the dispersibility of the obtained slurry composition of the present invention is improved.

Examples of the mixer may include ball mills, sand mills, pigment dispersing machines, kneaders, ultrasonic dispersion machines, homogenizers, planetary mixers, and Hobart mixers. Among them, ball mills, roll mills, pigment dispersing machines, kneaders, and planetary mixers are particularly preferably used since thereby dispersion at a high concentration is enabled.

The 50% volume cumulative diameter of the particles contained in the slurry composition of the present invention is preferably equal to or smaller than 35 μm, further preferably equal to or smaller than 25 μm. When the 50% volume cumulative diameter of the particles contained in the slurry composition of the present invention falls within the aforementioned range, high dispersion of the electroconductivity imparting material is obtained, and a homogeneous electrode can thus be obtained. Therefore, it is preferable that mixing by the aforementioned mixer is performed to a degree in which the 50% volume cumulative diameter of the particles contained in the slurry composition of the present invention falls within the aforementioned range.

[5. Electrode for Non-Aqueous Battery of the Invention]

The electrode for a non-aqueous battery of the present invention (appropriately referred to hereinbelow as “the electrode of the present invention”) includes a current collector, and an electrode active material layer provided on a surface of the current collector.

[5-1. Current Collector]

The material for the current collector is not particularly limited as long as the material has electrical conductivity and electrochemical durability. In view of having heat resistance, examples of the material may preferably include metal materials such as iron, copper, aluminum, nickel, stainless steel, titanium, tantalum, gold, and platinum. Among them, aluminum is particularly preferable as a material for the current collector for 4 positive electrode of a lithium secondary battery, and copper is particularly preferable as a material for the current collector for a negative electrode of a lithium secondary battery.

The shape of the current collector is not particularly limited. However, a sheet shape having a thickness of approximately 0.001 mm to 0.5 mm is preferable.

It is preferable that the surface of the current collector is subjected to a surface roughing treatment in advance of its use, for enhancing the adhesive strength of the electrode active material layer. Examples of the surface roughing method may include a mechanical polishing method, an electrolytic polishing method, and a chemical polishing method. In the mechanical polishing method, polishing cloth or paper having abrasive particles adhering thereon, grindstone, emery wheel, wire brush provided with, e.g., steel wire, or the like are used.

Further, in order to enhance the adhesion strength and electroconductivity of the electrode active material layer, an intermediate layer may be formed on a surface of the current collector.

[5-2. Electrode Active Material Layer]

The electrode active material layer is a layer containing at least an electrode active material. The electrode active material layer for the electrode of the present invention is produced by applying and drying the slurry composition of the present invention.

The method for applying the slurry composition of the present invention onto a current collector is not particularly limited. Examples of the method may include a doctor blade method, a dip method, a reverse roll method, direct roll method, a gravure method, an extrusion method, and a brushing method. As a result of the application of the slurry composition of the present invention onto the current collector, the solid content of the slurry composition of the present invention (for example, the electrode active material, the acrylic-based polymer, etc.) adheres to the surface of the current collector in a form of a layer.

After the application of the slurry composition of the present invention, the solid content of the slurry composition of the present invention adhering in a form of layer is then dried. Examples of the drying method may include drying by, e.g., warm air, hot air, and low humid air; vacuum drying; and drying by irradiation of, e.g., infrared rays, far infrared rays, and electron rays. The electrode active material layer is thereby formed on the surface of the current collector.

If necessary, heat treatment may be performed after the application of the slurry composition of the present invention. The heat treatment is usually performed at a temperature equal to or higher than 120° C. for 1 hour or longer.

Furthermore, it is preferable that the electrode active material layer is then subjected to pressurizing treatment using, for example, a mold press or a roll press. By performing the pressurizing treatment, the porosity of the electrode active material layer can be reduced. The porosity is preferably equal to or more than 5% and more preferably equal to or more than 7%, and preferably equal to or less than 15% and more preferably equal to or less than 13%. Too low porosity might cause difficulty in elevating volumetric capacity, and might also increase tendency to cause peel-off of the electrode active material layer to induce defects. Too high porosity might cause decrease in charging efficiency and discharging efficiency.

When the slurry composition of the present invention contains a curable polymer, it is preferable that the polymer is cured at an appropriate time after the application of the slurry composition of the present invention.

The thickness of the electrode active material layer at both the positive electrode and the negative electrode is usually equal to or thicker than 5 μm and preferably equal to or thicker than 10 μm, and usually equal to or thinner than 300 μm and preferably equal to or thinner than 250 μm.

[6. Non-Aqueous Battery of the Invention]

The non-aqueous battery of the present invention (appropriately referred to hereinbelow as “the battery of the present invention”) includes at least a positive electrode, a negative electrode, and a non-aqueous electrolyte solution, and usually further includes a separator. However, the battery of the present invention satisfies one or both of the following requirements (i) and (ii).

(i) The non-aqueous electrolyte solution is the electrolyte solution composition of the present invention.
(ii) One or both of the positive electrode and the negative electrode is the electrode of the present invention.

The non-aqueous battery of the present invention is usually a secondary battery, and may be, for example, a lithium secondary battery and a nickel metal hydride secondary battery, and is preferably a lithium secondary battery. Since the non-aqueous battery of the present invention satisfies one or both of the aforementioned requirements (i) and (ii), both high discharge capacity and stable charge-discharge cycle at high temperatures can be realized.

[6-1. Electrode]

In the battery of the present invention, the electrode of the present invention is used as one or both of the positive electrode and the negative electrode. The electrode of the present invention may be used as the positive electrode, the negative electrode, or both the positive and negative electrodes. Especially, it is preferable that the electrode of the present invention is a positive electrode since the acrylic-based polymer is suitable for the binder of the positive electrode, and the stable protective film formed by the ether compound of the present invention is presumed to be formed on the positive electrode.

When the electrode of the present invention is used as a positive electrode, the negative electrode may be the same as that of the aforementioned battery of the present invention, except that the acrylic-based polymer does not have be contained as the binder, and that the negative electrode does not have to be produced from the slurry composition containing the ether compound of the present invention. In this case, as the binder other than the acrylic-based polymer, a polymer is usually used. However, the specific type of the suitable binder varies depending on the type of the solvent which dissolves or disperses the binder in the binder composition.

For example, when an aqueous solvent is used as the solvent of the binder composition, examples of the binder may include a diene-based polymer, a fluorine-based polymer, and a silicon-based polymer. Among them, a diene-based polymer is preferable because of its excellent binding property with the electrode active material, and excellent strength and flexibility of the resulting electrode. The diene-based polymer is particularly suitable as the binder of the negative electrode because of its excellent reduction resistance and its ability to cause strong binding force.

A diene-based polymer is a polymer (diene-based polymer) which contains a monomer unit obtained by polymerizing conjugate dienes such as butadiene and isoprene. In the diene-based polymer, the ratio of the monomer unit obtained by the polymerization of the conjugate diene is usually equal to or higher than 40% by weight, preferably equal to or higher than 50% by weight, and more preferably equal to or higher than 60% by weight.

Examples of the diene-based polymer may include homopolymers of conjugate dienes such as polybutadiene and polyisoprene; copolymers of different types of conjugate dienes; and copolymers of conjugate dienes and a monomer copolymerizable with the conjugate diene. Examples of the copolymerizable monomer may include α,β-unsaturated nitrile compounds such as acrylonitrile and methacrylonitrile; unsaturated carboxylic acids such as acrylic acid and methacrylic acid; styrene-based monomers such as styrene, chlorostyrene, vinyltoluene, t-butylstyrene, vinylbenzoic acid, methyl vinylbenzoate, vinyl naphthalene, chloromethylstyrene, hydroxymethylstyrene, α-methylstyrene, and divinylbenzene; olefins such as ethylene and propylene; halogen atom-containing monomers such as vinyl chloride and vinylidene chloride; vinyl esters such as vinyl acetate, vinyl propionate, vinyl butyrate, and vinyl benzoate; vinyl ethers such as methyl vinyl ether, ethyl vinyl ether, and butyl vinyl ether; vinyl ketones such as methyl vinyl ketone, ethyl vinyl ketone, butyl vinyl ketone, hexyl vinyl ketone, and isopropenyl vinyl ketone; and heterocyclic ring-containing vinyl compounds such as N-vinylpyrrolidone, vinylpyridine, and vinylimidazole. As each of the conjugate diene and copolymerizable monomer, one species thereof may each be used alone, or two or mare species thereof may be used in combination at any ratio.

When a nonaqueous-based solvent is used as the solvent of the binder composition, examples of the binder may include fluorine-based polymers such as polytetrafluoroethylene (PTFE), polyvinylidenefluoride (PVDF), tetrafluoroethylene-hexafluoropropylene copolymer (FEP), vinylidene fluoride-based rubber, and tetrafluoroethylene-propylene rubber; vinyl-based polymers such as polyethylene, polypropylene, polyisobutylene, polyvinyl chloride, polyvinylidene chloride, polyvinyl acetate, polyvinyl alcohol, polyvinyl isobutyl ether, polyacrylonitrile, polymethacrylonitrile, allyl acetate, and polystyrene; diene-based polymers such as polybutadiene and polyisoprene; ether-based polymers containing a heteroatom in its main chain such as polyoxymethylene, polyoxyethylene, polycyclic thioether, and polydimethylsiloxane; condensed ester-based polymers such as polylactone polycyclic anhydride, polyethylene terephthalate, and polycarbonate; and condensed amide-based polymers such as nylon 6, nylon 66, poly-m-phenyleneisophthalamide, poly-p-phenyleneterephthalamide, and polypyromellitimide.

However, among the aforementioned binders, the binder which is enumerated as a binder suitable for an aqueous solvent may also be used in combination with a non-aqueous solvent, and the binder which is enumerated as a binder suitable for a non-aqueous solvent may also be used in combination with an aqueous solvent. For example, the aforementioned diene-based polymer may be used in combination with a non-aqueous solvent. Furthermore, as the binder, those having a crosslink structure may be used, and those into which a functional group is introduced by modification may also be used. As the binder, one species thereof may be used alone, or two or more species thereof may be used in combination at any ratio.

It is preferable that the glass transition temperature of the binder, the particle size in the case where the binder exists as particles, the amount of the binder, and the like usually fall within the same respective ranges as those for the acrylic-based polymer, for the same reason as that regarding the acrylic-based polymer.

When the battery of the present invention includes the electrolyte solution composition of the present invention as a non-aqueous electrolyte solution, both the positive electrode and the negative electrode may be an electrode other than the electrode of the present invention.

[6-2. Non-Aqueous Electrolyte Solution]

In the battery of the present invention, the electrolyte solution composition of the present invention is used as the non-aqueous electrolyte solution. However, when one or both of the positive electrode and the negative electrode of the battery of the present invention are the battery of the present invention, a non-aqueous electrolyte solution other than the electrolyte solution composition of the present invention may be used as the non-aqueous electrolyte solution.

[6-3. Separator]

The separator is a member provided between the positive electrode and the negative electrode for preventing short circuit of the electrodes. As the separator, a porous substrate having a pore portion is usually used. Examples of the separator may include (a) a porous separator having a pore portion, (b) a porous separator having a polymer coating layer formed on one or both of the surfaces thereof, and (c) a porous separator having a porous coating layer containing inorganic fillets or organic fillers formed thereon.

As the (a) porous separator having a pore, portion, for example, a porous film having a microscopic pore diameter, which does not have electron conductivity and has ionic conductivity and high resistance to organic solvents, is used. Specific examples of such a porous separator may include microporous films composed of a resin of a polyolefin-based polymer (for example, polyethylene, polypropylene, polybutene, and polyvinyl chloride), and a mixture or a copolymer thereof; microporous films composed of a resin such as polyethylene terephthalate, polycycloolefin, polyether sulfone, polyamide, polyimide, polyimidamide, polyaramid, polycycloolefin, nylon, and polytetrafluoroethylene; woven materials of a polyolefin-based fiber, or non-woven fabric thereof; and an aggregate of insulating material particles.

Examples of the (b) porous separator having a polymer coating layer formed on one or both of the surfaces thereof may include a polymer film for solid polymer electrolytes or gel polymer electrolytes such as polyvinylidene fluoride, polyethylene oxide, polyacrylonitrile, and a polyvinylidene fluoride-hexafluoropropylene copolymer; and a gelled polymer coating layer.

Examples of the (c) porous separator haying a porous coating layer containing inorganic fillers or organic fillets formed thereon may include a separator coated with a porous layer which is composed of inorganic fillers or organic fillers and a dispersant for the fillers.

Among them, a separator having a porous layer which is composed of inorganic fillers or organic fillers and the aforementioned dispersant for the fillers is preferable, since it can decrease the film thickness of the entire separator and increase the ratio of the active material in the battery, to thereby increase a capacity per volume.

The thickness of the separator is usually equal to or thicker than 0.5 μm and preferably equal to or thicker than 1 μm, and usually equal to or thinner than 40 μm, preferably equal to or thinner than 30 μm, and more preferably equal to or thinner than 10 μm. When the thickness falls within this range, resistance caused by the separator in the battery decreases, and workability during the production of a battery is excellent.

[6-4. Method for Producing Non-Aqueous Battery of the Invention].

The method for producing the non-aqueous battery of the present invention is not particularly limited. For example, the battery may be produced by stacking a negative electrode and a positive electrode with a separator interposed therebetween, rolling or folding the stack in conformity with a battery shape to be placed in a battery container, pouring the electrolyte solution composition of the present invention into the battery container, and then sealing the container. If necessary, an overcurrent prevention element such as an expanded metal, a fuse, and a PTC element; and lead plate may be provided, whereby an increase in internal pressure of the battery and overcharge and overdischarge can be prevented. The shape of the battery may be any of laminated cell type, coin type, button type, sheet type, cylindrical type, rectangular type, and flat type.

### EXAMPLES

Although the present invention will be specifically described with reference to the following examples, the invention shall not be limited to the following Examples, which may be arbitrary modified for implementation within a range not departing from the claims of the present invention and its equivalents. In the following description, part and % that represent an amount are on a weight basis, unless otherwise specified. Me represents a methyl group.

### Preparative Example 1

### Production of Ether Compound 1

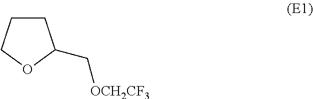

In a four-necked reaction vessel equipped with a condenser, a thermometer, and a dropping funnel, 30 g (0.29 mol) of tetrahydrofurfuryl alcohol and 32.6 g (0.32 mol) of triethylamine were dissolved in 300 ml of ethyl acetate in a nitrogen gas stream. Under ice bath cooling, 37.0 g (0.32 mol) of methanesulfonyl chloride was slowly added from the dropping funnel. Thereafter, reaction was carried out at room temperature for 1 hour.

After the reaction was completed, the product was washed with 0.1 N, aqueous HCl solution, and the obtained ethyl acetate layer was further washed with water. Anhydrous sodium sulfate was added to the ethyl acetate layer ter drying, and sodium sulfate was then removed by filtration. Under reduced pressure, ethyl acetate was distilled off using a rotary evaporator to obtain a pale yellow oil.

The entire amount of the obtained pale yellow oil and 29.4 g (0.29 mol) of 2,2,2-trifluoroethanol were dissolved in 300 ml of N,N-dimethylformamide. 61 g (0.44 mol) of potassium carbonate was added thereto, and reaction was carried out at 90° C. for 6 hours. 20 g (0.14 mol) of potassium carbonate was further added thereto, and reaction was carried out at 90° C. for 4 hours.

After the reaction was completed, the reaction mixture was poured into 1.5 liters of water, and extracted with 300 ml of ethyl acetate. To the ethyl acetate layer obtained by liquid separation, anhydrous sodium sulfate was added for drying, and sodium sulfate was then removed by filtration. Under reduced pressure, ethyl acetate was distilled off using a rotary evaporator to obtain 4.1 g (yield: 7.6%) of a pale yellow oil.

This pale yellow oil was purified by silica gel column chromatography (gradient hexane:ethyl acetate=from 90:10 to 85:15) to obtain 3.2 g of a pale yellow oil (yield: 6.0%). Furthermore, the obtained yellow oil was subjected to distillation under reduced pressure using a Kugelrohr apparatus in the presence of calcium hydride to obtain an ether compound 1 having a structure represented by the aforementioned formula (E1) as 2.1 g (yield: 3.7%) of a colorless oil.

The structure of the ether compound 1 was determined by ¹H-NMR and ¹³C-NMR. The obtained results are shown below.

¹H-NMR (400 MHz, CDCl₃, TMS, δ ppm): 4.08-4.03 (m, 1H), 3.95-1.83 (m, 3H), 3.79-3.74 (m, 1H), 3.69-3.65 (m, 1H), 3.60-3.56 (m, 1H), 2.00-1.83 (m, 3H), 1.66-1.59 (m, 1H).

¹³C-NMR (100 MHz, CDCl₃, δ ppm): 124.1 (q, J=277.5 Hz), 77.6, 75.0, 68.9 (q, J=34.3 Hz), 68.6, 27.8, 25.7.

### Preparative Example 2

### Production of Ether Compound 2

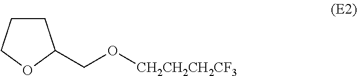

In a four-necked reaction vessel equipped with a condenser, a thermometer, and a dropping funnel, 1.0 g (25.2 mmol) of sodium hydride having a content ratio of 60 and 50 ml of dimethylformamide were charged in a nitrogen gas stream. The mixture was cooled in an ice bath, and 2.5 ml (25.2 mmol) of tetrahydrofurfuryl alcohol diluted with 10 ml of dimethylformamide was then slowly added thereto under ice bath cooling from the dropping funnel. Reaction was carried out at room temperature for 10 minutes, and then 5.0 g (0.64 mol) of 1,1,1-trifluoro-4-iodobutane diluted with 10 ml of dimethylformamide was slowly added thereto at room temperature froth the dropping funnel. Thereafter, reaction was carried out at 60° C. for 5 hours.

After the reaction was completed, 200 ml of water was poured into the reaction mixture, and extraction was performed twice with 200 ml of ethyl acetate. The ethyl acetate layer was dried with magnesium sulfate, and filtration was then performed for removing magnesium sulfate. The ethyl acetate layer was concentrated using a rotary evaporator to obtain a pale yellow oil.

This pale yellow oil was purified by silica gel column chromatography (hexane:ethyl acetate=9:1) to obtain 0.95 g of an ether compound 2 having a structure represented by the aforementioned formula (E2) as a colorless oil. Furthermore, the obtained pale yellow oil was subjected to distillation under reduced pressure using a Kugelrohr apparatus in the presence of calcium hydride to obtain 0.20 g 4.4%) of a colorless oil.

The structure of the ether compound 2 was determined by ¹H-NMR and ¹³C-NMR. The obtained results are shown below.

¹H-NMR (500 MHz, CDCl₃, TMS, δ ppm) 4.07-4.02 (m, 1H), 3.90-3.86 (m, 1H), 3.80-3.75 (m, 1H), 3.57-3.49 (m, 2H), 3.46 (dd, 1H, J=4.0 Hz, 10.5 H 3.43 (dd, 1H, J=6.0 Hz, 10.5 Hz), 2.25-2.15 (m, 2H), 1.99-1.81 (m, 5H), 1.63-1.56 (m, 1H).

¹³C-NMR (125 MHz, CDCl₃, δ ppm): 127.3 (q, J=275.5 Hz), 77.8, 73.7, 69.6, 68.4, 30.7 (q, J=28.8 Hz), 28.1, 25.7, 22.4 (t, J=2.7 Hz).

### Preparative Example Production of Ether Compound 3

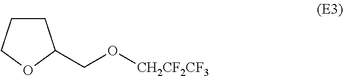

[Step 1: Production of Intermediate A]

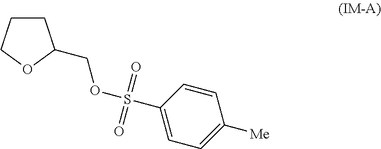 [SMILES: Cc1ccc(S(=O)(=O)OCC2CCCO2)cc1]

In a three-necked reaction vessel equipped with a thermometer, 117.9 g (1.154 mol) of tetrahydrofurfuryl alcohol, 200.0 g (1.049 mol) of para-toluenesulphonyl chloride, and 12.8 g (0.105 mol) of 4-dimethylaminopyridine were charged in a nitrogen gas stream. The mixture was dissolved in 1000 ml of tetrahydrofuran. This solution was cooled to 0° C., and 127.4 g (1.259 mol) of triethylamine was added dropwise thereto over 30 minutes. Thereafter, the reaction mixture was warmed back to room temperature, and reacted for 1 hour. Then, the reaction mixture was reacted under heat reflux conditions for 5 hours.

After the reaction was completed, the reaction mixture was cooled back to room temperature, and concentrated using a rotary evaporator so that the amount of the reaction solvent tetrahydrofuran became approximately 300 ml. Then, 1000 ml of distilled water and 300 ml of a saturated salt solution were added thereto, and the mixture was extracted with 1000 ml of ethyl acetate. The organic layer was dried with sodium sulfate, and concentrated by a rotary evaporator. Then, the obtained product was purified by silica gel column chromatography (hexane:tetrahydrofuran=1:2) to obtain 250.1 g of an intermediate A having a structure represented by the aforementioned formula (IM-A) with a yield of 93%.

The structure of the intermediate A was determined by ¹H-NMR. The obtained results are shown below.

¹H-NMR (500 MHz, CDCl₃, TMS, δ ppm) δ 7.80 (d, 2H, =8.0 Hz), 7.34 (d, 2H, J=8.0 Hz), 4.06-4.11 (m, 1H), 3.96-4.03 (m, 2H), 3.70-3.81 (m, 2H), 2.45 (s, 3H), 1.94-2.01 (m, 1H), 1.82-1.91 (m, 2H), 1.62-1.70 (m, 1H).

[Step 2: Production of Ether Compound 3]

In a four-necked reaction vessel equipped with condenser, a thermometer, and a dropping funnel, 5.0 g (19.5 mmol) of the intermediate A, 2.9 ml (29.3 mmol) of 2,2,3,3,3-pentafluoro-1-propanol, 50 ml of dimethylformamide, and 8.1 g (58.5 mmol) of potassium carbonate were charged in a nitrogen gas stream. Thereafter, reaction was carried out at room temperature for 18 hours.

After the reaction was completed, potassium carbonate was removed by filtration. The filtrate liquid was poured into 100 ml of water, and extraction was performed three times with 100 ml of chloroform. The chloroform layer was dried with magnesium sulfate, and filtration was then performed for removing magnesium sulfate. The chloroform layer was concentrated using a rotary evaporator to obtain a pale yellow oil.

This pale yellow oil was purified by silica gel column chromatography (hexane:ethyl acetate=75:25) to obtain 1.65 g of an ether compound 3 having a structure represented by the aforementioned formula (E3) as a colorless oil. Furthermore, the obtained pale yellow oil was subjected to distillation under reduced pressure using a Kugelrohr apparatus in the presence of calcium hydride to obtain 0.30 g (yield: 6.6%) of a colorless oil.

The structure of the ether compound 3 was determined by ¹H-NMR and ¹³C-NMR. The obtained results are shown below.

¹H-NMR (500 MHz, CDCl₃, TMS, δ ppm): 4.09-3.93 (m, 3H), 3.89-3.85 (m, 1H), 3.80-3.76 (m, 1H), 3.67 (dd, 1H, J=3.5 Hz, 10.5 Hz), 3.60 (dd, 1H, J=5.5 Hz, 10.5 Hz), 2.00-1.83 (m, 3H), 1.69-1.62 (m, 1H).

¹³C-NMR (125 MHz, CDCl₃, 6 ppm): 118.7 (qt, J=35.0, 285.0 Hz), 113.1 (tq, J=36.3 Hz, 253.8 Hz), 77.9, 75.1, 68.5, 68.0 (t, J=256.3 Hz), 27.7, 25.5.

### Examples 1 to 5 and Comparative Examples 1 to 3

### Production and Evaluation of Half Cell Battery

[Production of Binder Composition (Acrylic-Based Polymer 1)]

In a polymerization tank A, 10.78 parts of 2-ethylhexyl acrylate, 1.25 parts of acrylonitrile, 0.12 parts of sodium laurylsulfate, and 40.0 parts of ion exchanged water were charged. Furthermore, 0.2 parts of ammonium persulfate as a polymerization initiator and 10 parts of ion exchanged water were added thereto, and the mixture was warmed to 60° C. and stirred for 90 minutes. In another polymerization tank B, 67.11 parts of 2-ethylhexyl acrylate, 18.65 parts of acrylonitrile, 2.01 parts of methacrylic acid, 0.2 parts of allyl methacrylate, 0.7 parts of sodium laurylsulfate, and 88 parts of ion exchanged water were charged, and stirred to prepare an emulsion. The emulsion prepared in the polymerization tank B was sequentially added from the polymerization tank B to the polymerization tank A over approximately 180 minutes, and the mixture was stirred for approximately 120 minutes. When the amount of monomer consumption reached 95%, the mixture was cooled to terminate the reaction. A dispersion liquid 1 in which particles of an acrylic-based polymer 1 were dispersed was thereby obtained. The polymerization conversion ratio calculated from the solid content concentration was 92.6%. The solid content concentration of the obtained dispersion liquid 1 was 36.7%. Furthermore, the glass transition temperature Tg of the acrylic-based polymer 1 was −35.4° C.

[1B. Production of Binder Composition (Acrylic-Based Polymer 2)]

In a polymerization tank A, 2.0 parts of itaconic acid, 0.1 parts of sodium alkyl diphenyl ether disulfonate (manufactured by Dow Chemical Company: Dowfax 2A1), and 76.0 parts of ion exchanged water were charged. Furthermore, 0.6 parts of potassium persulfate as a polymerization initiator and 10 parts of ion exchanged water were added, and the mixture was warmed to 80° C. and stirred for 90 minutes. In another polymerization tank B, 76 parts of 2-ethylhexyl acrylate, 2.0 parts of acrylonitrile, 2.0 parts of itaconic acid, 0.6 parts of sodium alkyl diphenyl ether disulfonate, and 60 parts of ion exchanged water were charged and stirred to prepare an emulsion. The emulsion prepared in the polymerization tank B was sequentially added from the polymerization tank B to the polymerization tank A over approximately 180 minutes, and the mixture was stirred for approximately 120 minutes. After the amount of monomer consumption reached 95%, 0.2 parts of ammonium persulfate and 5 parts of ion exchanged water were added, and the mixture was warmed to 90° C. and stirred for 1.20 minutes. Then, the mixture was cooled to terminate the reaction to obtain a dispersion liquid 2 in which particles of an acrylic-based polymer 2 were dispersed in water. The polymerization conversion ratio calculated from the solid content concentration was 92.3%. The solid content concentration of the obtained dispersion liquid 2 was 38.8%. Furthermore, the glass transition temperature Tg of the acrylic-based polymer 2 was −37.0° C.

[1C. Preparation of Carboxymethyl Cellulose Aqueous Solution 1]

As an aqueous solution 1 of carboxymethyl cellulose (appropriately referred to hereinbelow as “CMC”) (CMC aqueous solution 1), an aqueous solution was prepared by adding water to carboxymethyl cellulose (trade name “BS-H”, manufactured by Dai-ichi Kogyo Seiyaku Co., Ltd.) so that the solid content concentration was adjusted to 2%.

[1D. Production of Slurry Composition for Positive Electrodes]

Using a planetary mixer, 100 parts of LiMn₂O₄ as a positive electrode active material and 5 parts of acetylene black as a electroconductivity imparting material were mixed. To the obtained mixture, the CMC aqueous solution 1 (solid content concentration 2%) in the amount of 0.8 parts in terms, of CMC amount was added, and mixing was performed for 60 minutes. The mixture was diluted with 5.5 ml of water, and the dispersion liquid 1 (solid content concentration 36.7%) containing the acrylic-based polymer 1 obtained in the aforementioned 1A was added so that the added amount of the acrylic-based polymer 1 was 1.0 part, or the dispersion liquid 2 (solid, content concentration 38.3%) containing the acrylic-based polymer 2 obtained in the aforementioned 1B was added so that the added amount of the acrylic-based polymer 2 was 1.0 part, as an amount. Then mixing was performed for 10 minutes. This was subjected to a defoaming treatment to obtain a slurry composition for positive electrodes which was glossy and had a good fluidity.

[1E. Production of Positive Electrodes]

The slurry composition for positive electrodes obtained in the aforementioned 1D was applied onto an aluminum foil having a thickness of 18 μm using a 75 μm doctor blade, and dried at 50° C. for 20 minutes. Thereafter, drying was further performed at 110° C. for 20 minutes. The electrode thus prepared was subjected to roll press to obtain a positive electrode having an electrode active material layer having a thickness of 50 μm. The positive electrode thus prepared was dried at 105° C. for 3 hours immediately prior to production of a battery, and then used.

[1F. Production of Electrolyte Solution]

An electrolyte solution (manufactured by Kishida Chemical Co., Ltd.) in which LiPF₆ was dissolved at a concentration of 1 mol/L in a mixed solvent of ethylene carbonate/diethyl carbonate=1/2 (volume ratio) was prepared. In a glove box, each of the ether compounds 1 to 3 synthesized in Preparative Examples 1 to 3 in an amount of 0.15 ml was added to 10 ml of the electrolyte solution, and then stirred. Each of the solutions thus obtained was used as an electrolyte solution composition for performing the battery evaluation test which will be described later. The composition to which the ether compound 1 was added, the composition in which the ether compound 2 was added, and the composition in which the ether compound 3 was added were designated as electrolyte solution compositions 1 to respectively.

For comparison, there were also prepared an electrolyte solution composition C1 produced in the same manner as in the electrolyte solution compositions 1 to 3 except that any of the ether compounds 1 to 3 produced in Preparative Examples 1 to 3 was net added; an electrolyte solution composition C2 produced in the same manner as in the electrolyte solution compositions 1 to 3 except that tetrahydrofuran was used in place of the ether compounds 1 to 3 produced in Preparative Examples 1 to 3; and an electrolyte solution composition C3 produced in the same manner as in the electrolyte solution compositions 1 to 3 except that 2-methyltetrahydrofuran was used in place of the ether compounds 1 to 3 produced in Preparative Examples 1 to 3.

[1G. Preparation of Coin Cell Battery for Evaluation]

The positive electrode obtained in the aforementioned procedure was cut out in a disc shape having a diameter of 12 mm. A counterelectrode thereof was prepared by cutting out lithium metal in a disc, shape having a diameter of 14 mm. A single layer polypropylene separator (porosity: 55%) having a thickness of 25 μm was produced by dry method. This was cut in a disc shape having a diameter of 19 mm, to prepare a separator.

The disc-shaped positive electrode, the disc shaped separator, and the disc-shaped lithium metal were arranged, and a stainless steel plate having a thickness of 0.5 mm was placed thereon. Furthermore, an expanded metal was placed thereon. These were placed in a stainless steel coin-type outer container equipped with a polypropylene packing (diameter 20 mm, height 1.8 mm, stainless steel thickness 0.25 mm). The positional relationship of these was as described below. That is, the aluminum foil of the disc-shaped positive electrode was in contact with a bottom surface of the outer container. The disc-shaped separator was interposed between the disc-shaped positive electrode and the disc-shaped lithium metal. The surface on the electrode active material layer side of the positive electrode was opposed to the disc-shaped lithium metal via the disc-shaped separator. The stainless steel plate was placed on the lithium metal. The expanded metal was placed on the stainless steel plate. Thereafter, one of the aforementioned electrolyte solution compositions was poured into the container so that air did not remain therein, and the battery can was sealed. Thus, a coin cell (coin cell CR2032) that is a lithium ion secondary battery having a diameter of 20 mm and a thickness of approximately 3.2 mm was prepared.

Each combination of the dispersion liquid of the acrylic-based polymer and the electrolyte solution composition which were used in Examples 1 to 5 and Comparative Examples 1 to 3 was as shown in Table 1.

[1H. Battery Properties: Evaluation of Cycle Property]

Each of 10 coin cell batteries was charged up to 4.8 V by a constant current method at 0.2 C at 23° C., and then discharged down at 0.2 C to 30 V. Subsequently, charging and discharging were repeated under an atmosphere of 60° C. wherein charging was performed by a constant current method at 0.5 C to 4.3V and discharging was performed at 1.0 C to 3.0 V, whereby the electrical capacity was measured. The mean value of 10 cells was taken as a measurement value. The capacity maintenance ratio represented by the ratio (%) of the discharge capacity, after completion of 100 cycles and the discharge capacity after completion of 1 cycle under an atmosphere of 60° C. was calculated. High capacity maintenance ratio is considered to represent excellent high temperature cycle property.

The obtained evaluation results are summarized in Table 1.

### Examples 6 to 9 and Comparative Examples 4 to 6

### Production and Evaluation of Full Cell Battery

[6A. Production of Binder Composition for Negative Electrode]

In a 5 MPa pressure-resistant container equipped with a stirrer, 49 parts of 1,3-butadiene, 3.3 parts of methacrylic acid, 0.5 parts of acrylic acid, 46.7 parts of styrene, 0.27 parts of tert-dodecylmercaptan as a chain transfer agent, 2.52 parts of soft type sodium decylbenzenesulphonate as an emulsifier, 150 parts of ion exchanged water, and 0.5 parts of potassium persulfate as a polymerization initiator were placed, and sufficiently stirred. Then, the mixture was warmed to 50° C., and polymerization was initiated. When the polymerization conversion ratio reached 960, the product was cooled to terminate the reaction to obtain an aqueous dispersion liquid containing a binder.

To the aforementioned aqueous dispersion liquid containing a binder, 5% sodium hydroxide aqueous solution was added for adjusting the pH to 8. The unreacted monomer was then removed by heat distillation under reduced pressure, and the residue was cooled to 30° C. or lower to obtain a binder composition for a negative electrode.

[6B. Preparation of CMC Aqueous Solution 2]

As CMC aqueous solution 2, an aqueous solution was prepared by adding water to carboxymethyl cellulose (trade name “MAC350HC”, from Nippon Paper Chemicals Co., Ltd.) so that the solid content concentration was adjusted to 1%.

[60. Production of Slurry Composition for Secondary Battery Negative Electrodes]

In a planetary mixer, 100 parts of artificial graphite (mean particle size: 24.5 μm) having a specific surface area of 4 m²/g as a negative electrode active material, and 0.64 parts (based on solid content) of the aforementioned CMC aqueous solution 2 were charged. The solid content concentration was adjusted to 59% with water. Then mixing was performed at 25° C. for 60 minutes. Subsequently, 0.36 parts (based on solid content) of the CMC aqueous solution 2 was added thereto. The solid content concentration was adjusted to 47% with water, and mixing was further performed at 25° C. for 15 minutes to obtain a mixture solution.

To the mixture solution, 1 part (based on solid content) of the aforementioned binder composition for a negative electrode and water were added to adjust the final solid content concentration to 45%, and then mixing was performed for additional 10 minutes. This mixture was subjected to a defoaming treatment under reduced pressure to obtain a slurry composition for the secondary battery negative electrode having good fluidity.

[6D. Production of Negative Electrode]

The aforementioned slurry composition for secondary battery negative electrodes was applied onto a copper foil having a thickness of 20 μm using a 50 μm doctor blade, and dried at 50° C. for 20 minutes. Thereafter, drying was further performed at 110° C. for 20 Minutes. The electrode thus prepared was subjected to roll press to obtain a negative electrode having an electrode active material layer having a thickness of 50 μm. The negative electrode thus prepared was dried at 60° C. for 10 hours immediately prior to production of a battery, and then used.

[6E. Production of Electrolyte Solution]

An electrolyte solution (manufactured by Kishida Chemical Co., Ltd.) in which LiPF₆ was dissolved at a concentration of 1 mol/L in a mixed solvent of ethylene carbonate/diethyl carbonate=1/2 (volume ratio) and vinylene carbonate (1.5% by volume) was prepared. In a glove box, one of the compound 1 synthesized in Preparative Example 1, the compound 3 synthesized in Preparative Example 3, and a compound for comparisons (the same as that used in 1F of Example a) in an amount of 0.15 ml was added, or nothing was added, to 10 ml of the electrolyte solution, and then stirred. Each of the electrolyte solution compositions thus obtained was used as an electrolyte solution composition for performing the battery evaluation test which will be described later.

[6F. Preparation of Coin Cell Battery for Evaluation]

The positive electrode obtained in 1E of Examples 1 to 3 was cut out in a disc shape having a diameter of 12 mm. A counterelectrode thereof was prepared by cutting out the negative electrode obtained in the aforementioned 6D in a disc shape having a diameter of 16 mm. A single layer polypropylene separator (porosity: 55%) having a thickness of 25 μm was produced by dry method. This was cut in a disc shape having a diameter of 19 mm, to prepare a separator.

The disc-shaped positive electrode, the disc-shaped separator, and the disc-shaped negative electrode were arranged, and a stainless steel plate having a thickness of 1.0 mm was placed thereon. Furthermore, an expanded metal was placed thereon. These were placed in a stainless steel coin-type outer container equipped with a polypropylene packing (diameter 20 mm, height 1.8 mm, stainless steel thickness 0.25 mm). The positional relationship of these was as described below. That is, the aluminum foil of the disc-shaped positive electrode was in contact with a bottom surface of the outer container. The disc-shaped separator was interposed between the disc-shaped positive electrode and the disc-shaped negative electrode. The positive electrode was arranged such that the surface on the side of its electrode active material layer was in contact with the disc-shaped separator. The negative electrode was also arranged such that the surface on the side of its electrode active material layer was in contact with the disc-shaped separator. The stainless steel plate was placed on the copper foil of the negative electrode. The expanded metal was placed on the stainless steel plate. Thereafter, one of the aforementioned electrolyte solution compositions was poured into the container so that air did not remain therein, and the battery can was sealed. Thus, a coin cell (coin cell CR2032) that is a lithium ion secondary battery having a diameter of 20 mm and a thickness of approximately 3.2 mm was prepared. Each combination of the dispersion liquid of the acrylic-based polymer and the electrolyte solution composition which were used in Examples 6 to 9 and Comparative Examples 4 to 6 was as shown in Table 1.

[6G. Battery Properties: Evaluation of Cycle Property]

As to the batteries thus obtained, evaluation was performed in the same manner as in 1H of Examples 1 to 5. The obtained evaluation results are summarized in Table 1.

[Table 1. Results of Example 1 to 9 and Comparative
Examples 1 to 6]

| | | | | | Discharge | |
|---|---|---|---|---|---|---|
| | | | | | capacity | Capacity |
| | | | Half | | after | main- |
| | | Positive | cell/ | Initial | 100 | tenance |
| | Ether | electrode | full | capacity | cycles | ratio |
| | compound | binder | cell | (mAh/g) | (mAh/g) | (%) |
| Ex. 1 | Ether | Acrylic | Half | 103.64 | 99.86 | 96.4 |
| | compound 1 | polymer 1 | cell | | | |
| Ex. 2 | Ether | Acrylic | Half | 103.33 | 99.88 | 96.7 |
| | compound 2 | polymer 1 | cell | | | |
| Ex. 3 | Ether | Acrylic | Half | 103.83 | 99.98 | 96.3 |
| | compound 3 | polymer 1 | cell | | | |
| Ex. 4 | Ether | Acrylic | Half | 103.56 | 99.97 | 96.5 |
| | compound 1 | polymer 2 | cell | | | |
| Ex. 5 | Ether | Acrylic | Half | 103.18 | 100.00 | 96.9 |
| | compound 3 | polymer 2 | cell | | | |
| Ex. 6 | Ether | Acrylic | Full | 80.27 | 72.56 | 90.4 |
| | compound 1 | polymer 1 | cell | | | |
| Ex. 7 | Ether | Acrylic | Full | 80.73 | 73.89 | 91.5 |
| | compound 3 | polymer 1 | cell | | | |
| Ex. 8 | Ether | Acrylic | Full | 80.05 | 72.05 | 90.0 |
| | compound 1 | polymer 2 | cell | | | |
| Ex. 9 | Ether | Acrylic | Full | 80.34 | 72.45 | 90.2 |
| | compound 3 | polymer 2 | cell | | | |
| Comp. | None | Acrylic | Half | 102.89 | 96.30 | 93.6 |
| Ex. 1 | | polymer 1 | cell | | | |
| Comp. | Tetrahydro- | Acrylic | Half | 103.22 | 94.53 | 91.6 |
| Ex. 2 | furan | polymer 1 | cell | | | |
| Comp. | 2-Methyl- | Acrylic | Half | 102.92 | 96.54 | 93.8 |
| Ex. 3 | tetrahydro- | polymer 1 | cell | | | |
| | furan | | | | | |
| Comp. | None | Acrylic | Full | 77.18 | 66.53 | 86.2 |
| Ex. 4 | | polymer 1 | cell | | | |
| Comp. | Tetrahydro- | Acrylic | Full | 77.79 | 68.55 | 88.1 |
| Ex. 5 | furan | polymer 1 | cell | | | |
| Comp. | 2-Methyl- | Acrylic | Full | 78.65 | 69.08 | 87.8 |
| Ex. 6 | tetrahydro- | polymer 1 | cell | | | |
| | furan | | | | | |

From the results in Table 1, it was found out that the batteries in which an electrolyte solution composition containing the ether compound of the present invention was used as an electrolyte solution had high capacity maintenance ratio at a temperature as high as 60° C. and had excellent cycle property. Examples 1 to 5 resulted in excellent initial capacity to a degree equal to or higher than those of Comparative Examples 1 to 3, as well as in high discharge capacity after 100 cycles. Examples 6 to 9 resulted in excellent initial capacity to a degree equal to or higher than those of Comparative Examples 4 to 6, as well as in high discharge capacity after 100 cycles. From these results, it was confirmed that a battery containing the ether compound of the present invention can exhibit a high discharge capacity, and can realize a high discharge capacity and a stable charge-discharge cycle under a high temperature environment in a balanced manner.

### INDUSTRIAL APPLICABILITY

The ether compound of the present invention, can be used, for example, as an additive of a non-aqueous battery electrolyte solution, a binder composition for a non-aqueous battery electrode, a slurry composition for a non-aqueous battery electrode, and the like.

The electrolyte solution composition, binder composition, slurry composition, and electrode of the present invention can be applied to, for example, a secondary battery such as a lithium secondary battery.

The battery of the present invention can be used as, for example, power sources for electrical equipment such as cell phones and notebook computers and vehicles such as electric vehicles.

1. An ether compound represented by the following formula (1), 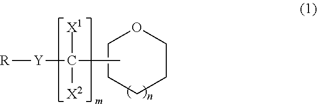
wherein
n represents 0 or 1,
m represents an integer of 0 to 2,
Y represents any one selected from the group consisting of —O—, —S—, —C(═O)—O—, and —O—C(═O)—,
X¹ and X² each independently represent a hydrogen atom or a fluorine atom, and
R represents an aliphatic hydrocarbon group having 1 to 20 carbon atoms and substituted with one or more fluorine atoms, with a proviso that, when m is 0, R has 3 to 20 carbon atoms; wherein R may have in the bond thereof one or more intervening moieties selected from the group consisting of an oxygen atom, a sulfur atom, and a carbonyl group.

2. The ether compound according to claim 1, wherein the ether compound is represented by the following formula (2), 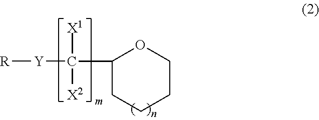
wherein
n represents 0 or 1,
m represents an integer of 0 to 2,
Y represents any one selected from the group consisting of —O—, —S—, —C(═O)—O—, and —O—C(═O)—,
X¹ and X² each independently represent a hydrogen atom or a fluorine atom, and
R represents an aliphatic hydrocarbon group having 1 to 20 carbon atoms and substituted with one or more fluorine atoms, with a proviso that, when m is 0, R has 3 to 20 carbon atoms; wherein
R may have in the bond thereof one or more intervening moieties selected from the group consisting of an oxygen atom, a sulfur atom, and a carbonyl group.

3. The ether compound according to claim 2, wherein the ether compound is represented by the following formula (3), 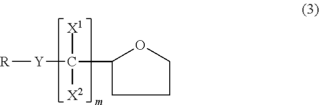
wherein
m represents an integer of 0 to 2,
Y represents any one selected from the group consisting of —O—, —S—, —C(═O)—O—, and —O—C(═O)—,
X¹ and X² each independently represent a hydrogen atom or a fluorine atom, and
R represents an aliphatic hydrocarbon group having 1 to 20 carbon atoms and substituted with one or more fluorine atoms, with a proviso that, when m is 0, R has 3 to 20 carbon atoms; wherein
R may have in the bond thereof one or more intervening moieties selected from the group consisting of an oxygen atom, a sulfur atom, and a carbonyl group.

4. An electrolyte solution composition for a non-aqueous battery, comprising an organic solvent, an electrolyte dissolved in the organic solvent, and the ether compound according to claim 1.

5. A binder composition for a non-aqueous battery electrode, comprising an acrylic-based polymer and the ether compound according to claim 1.

6. A slurry composition for a non-aqueous battery electrode, containing an electrode active material and the binder composition for a non-aqueous battery electrode according to claim 5.

7. An electrode for a non-aqueous battery, comprising a current collector and an electrode active material layer provided on a surface of the current collector,
wherein the electrode active material layer is obtained by applying and drying the slurry composition for a non-aqueous battery electrode according to claim 6.

8. A non-aqueous battery, comprising a positive electrode, a negative electrode, and a non-aqueous electrolyte solution,
wherein the non-aqueous electrolyte solution is the electrolyte solution composition for a non-aqueous battery according to claim 4.

9. A non-aqueous battery, comprising a positive electrode, a negative electrode, and a non-aqueous electrolyte solution,
wherein one or both of the positive electrode and the negative electrode is the electrode for a non-aqueous battery according to claim 7.
